(19) United States
(12) Patent Application Publication
(10) Pub. No.: US 2023/0149674 A1
(43) Pub. Date: May 18, 2023

(54) EXPANDABLE INTRODUCER FOR DILATING THE DISTAL TIP OF AN INTRODUCER SHEATH

(71) Applicant: Edwards Lifesciences Corporation, Irvine, CA (US)

(72) Inventors: Yair A. Neumann, Moshav Sede Varburg (IL); Russell T. Joseph, Las Flores, CA (US); Orna Popper, Zichron Yaakov (IL); Izaak Rosen, Irvine, CA (US); Tiffany Diemtrinh Tran, Anaheim, CA (US)

(21) Appl. No.: 18/097,365

(22) Filed: Jan. 16, 2023

Related U.S. Application Data
(63) Continuation of application No. PCT/US2021/041991, filed on Jul. 16, 2021.
(60) Provisional application No. 63/053,506, filed on Jul. 17, 2020.

(51) Int. Cl. A61M 29/02; A61M 25/10; A61F 2/24

(52) U.S. Cl. CPC A61M 29/02; A61M 25/1002; A61F 2/2433; A61M 2025/1079; A61M 2025/1061; A61M 2025/1081

(57) ABSTRACT

Expandable sheaths and introducers are disclosed herein. In some examples, the expandable introducer includes an elongated body member, an inflatable balloon disposed between a proximal and distal end of the elongated body member, and an inflation lumen in fluid communication with the inflatable balloon. A portion of the balloon is sized and configured to pass through a distal opening of an expandable introducer sheath when in the deflated condition, and to dilate the distal end of the introducer sheath as the balloon expands from the deflated configuration to the inflated configuration. Methods of making and using the devices disclosed herein are also disclosed.

This application is a continuation of a PCT Application No. PCT/US2021/041991, filed Jul. 16, 2021, entitled EXPANDABLE INTRODUCER FOR DILATING THE DISTAL TIP OF AN INTRODUCER SHEATH, which claims the benefit of U.S. Provisional Patent Application No. 63/053,506, filed Jul. 17, 2020, entitled EXPANDABLE INTRODUCER FOR DILATING THE DISTAL TIP OF AN INTRODUCER SHEATH, where each of above-referenced application is incorporated herein by reference in its entirety.

### FIELD

The present application relates to expandable introducer sheaths for the delivery of prosthetic devices such as transcatheter heart valves, and methods of making the same.

### BACKGROUND

Endovascular delivery catheter assemblies are used to deliver surgical devices and prosthetic implants, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature along with an introducer, and a housing that contains one or more sealing valves that allow a delivery apparatus to be placed in fluid communication with the vasculature with minimal blood loss. Such introducer sheaths may be radially expandable. However, such sheaths tend to have complex mechanisms, such as ratcheting mechanisms that maintain the sheath in an expanded configuration once a device with a larger diameter than the sheath's original diameter is introduced. Moreover, these introducer sheaths often include a compressed distal tip that is relatively stiffer than the remainder of the sheath body. The narrow and stiff distal tip can be formed by compressing and heating bonding the material of the sheath tip. While the bond between the material layers and/or the folded/compressed material is configured to break during passage of a medical device, a high push force is required causing possible damage to the medical device and risking trauma to the patient. Additionally, in some procedures, retraction of the medical device for replacement/repositioning and/or removal of other equipment is too large to fit through the distal opening of the sheath. For example, in the case of pre-implantation balloon aortic valvuloplasty (pre-BAV), the device passing through the tip is provided with a smaller diameter than that of the tip opening. A balloon may be delivered through the introducer sheath in a deflated state, having a diameter smaller than that of the sheath's tip (e.g., 10 Fr) such that the tip does not dilate when the balloon is passed distally there-through. After the pre-BAV procedure is complete, the diameter of the (deflated) balloon may be larger than that of the compressed tip, such that retraction of the balloon may face difficulties. Accordingly, there remains a need in the art for an improved introducer sheath for endovascular systems used for implanting valves and other prosthetic devices.

### SUMMARY

The expandable introducer disclosed herein includes: an elongated body member; an inflatable balloon disposed between a proximal and distal end of the elongated body member, the balloon expandable from a deflated configuration to an inflated configuration; and an inflation lumen in fluid communication with the inflatable balloon, the inflation sized and configured for providing an inflation fluid to the balloon. In the deflated configuration, an outer diameter of the balloon corresponds to an outer diameter of the elongated body member, and in the inflated configuration, the outer dimeter of the balloon is greater than the outer diameter of the elongated body member, and at least a portion of the balloon is sized and configured to pass through a distal opening of an expandable introducer sheath when the balloon is in the deflated condition, the balloon sized and configured to expand at least a portion of a distal end of an introducer sheath as the balloon is inflated.

Another expandable introducer sheath system disclosed herein includes: an expandable introducer sheath for deploying a medical device; an introducer received within a central lumen of the introducer sheath and axially and rotatably movable therein, the introducer comprising: an elongated body member having a proximal end and a tapered distal end; an inflatable balloon disposed between the proximal end and the distal end of the elongated body member, the balloon expandable from a deflated configuration to an inflated configuration; and an inflation lumen in fluid communication with the inflatable balloon, the inflation sized and configured for providing an inflation fluid to the balloon. In the deflated configuration, an outer diameter of the balloon corresponds to an outer diameter of the elongated body member, and in the inflated configuration, the outer dimeter of the balloon is greater than the outer diameter of the elongated body member. At least a portion of the balloon is sized and configured to pass through a distal opening of the introducer sheath when the balloon is in the deflated condition, as the balloon is inflated at least a portion of the distal end of an introducer sheath expands, increasing a diameter of the distal opening.

Methods of pre-dilating an introducer sheath tip are also disclosed herein. One example of pre-dilating an introducer sheath tip includes: positioning an expandable introducer within a central lumen of an expandable sheath, the introducer including: an elongated body member; an inflatable balloon disposed between a proximal and distal end of the elongated body member, the balloon expandable from a deflated configuration to an inflated configuration, when in the deflated configuration an initial diameter of the balloon corresponds to an outer diameter of the elongated body member, when in the inflated configuration, the inflated dimeter of the balloon is greater than the outer diameter of the elongated body member; and an inflation lumen in fluid communication with the inflatable balloon, the inflation sized and configured for providing an inflation fluid to the balloon. The method further includes advancing the introducer axially within the central lumen of the sheath such that the inflatable balloon is axially aligned with a distal opening of the sheath; inflating the balloon to the inflated diameter, where the inflated diameter of the balloon is greater than an initial diameter of the distal opening and thereby expanding a diameter of the distal opening of the sheath; deflating the balloon; and withdrawing the introducer from the central lumen of the sheath.

Methods of delivering a medical device using an expandable introducer are also disclosed herein. One example includes, inserting an expandable sheath and an expandable introducer at least partially into the vasculature of the patient, the introducer received within a central lumen of the sheath; advancing the introducer axially within the central lumen of the sheath such that an inflatable balloon disposed on an elongated body member of the introducer is axially aligned with a distal opening of the sheath; inflating the balloon to a diameter greater than an initial diameter of the distal opening and thereby expanding a diameter of the distal opening of the sheath; deflating the balloon; withdrawing the introducer from the central lumen of the sheath; advancing a medical device through the central lumen of the sheath; and delivering the medical device to the patient.

An expansion device that is configured to be received within an expandable sheath is also disclosed herein. The expansion device includes a body and a radially extending protrusion. The body includes an outer surface, a proximal end, and a tapered distal end opposite and spaced apart from the proximal end of the body. The radially extending protrusion is disposed along a portion of the body and includes an outer surface. The radially extending protrusion has a diameter greater than a diameter of the body. The device is sized and configured to be received within a central lumen of an expandable sheath such that the radially extending protrusion at least partially expands a portion of the expandable sheath.

Also disclosed herein is a sheath system. The sheath system includes an expandable sheath and an expansion device. The expandable sheath includes an inner layer defining a central lumen of the sheath and an outer layer extending at least partially around the inner layer. The inner layer and outer layer transition from a non-expanded and an expanded configuration. The expansion device is movable within the central lumen of the sheath. The expansion device includes a body and a radially extending protrusion. The body includes an outer surface, a proximal end and a tapered distal end opposite and spaced apart from the proximal end. The radially extending protrusion is disposed along a portion of the body and has an outer surface having a diameter greater than a diameter of the body. Receipt of the expansion device within the central lumen of the sheath causes the sheath to locally expand at least a portion of the sheath in response to the outwardly directed radial force provided by the radially extending protrusion.

A method of locally expanding an expandable sheath is also disclosed. The method includes introducing an expansion device within a central lumen of the expandable sheath; introducing the combined expandable sheath and expansion device into a patient's vascular; and advancing the expansion device distally within the central lumen of the expandable sheath to locally expand the lumen of the sheath at a local axial location corresponding to an axial location of a radially extending protrusion provided on the expansion device.

Another method of locally expanding an expandable sheath is also disclosed. The method includes introducing an expandable sheath into a patient's vascular, the expandable sheath having a central lumen; introducing the expansion device into the central lumen of the expandable sheath; and advancing the expansion device distally within the central lumen of the expandable sheath to locally expand the lumen of the sheath at a local axial location corresponding to an axial location of a radially extending protrusion provided on the expansion device.

### BRIEF DESCRIPTION OF THE DRAWINGS

Example features and implementations are disclosed in the accompanying drawings. However, the present disclosure is not limited to the precise arrangements shown, and the drawings are not necessarily drawn to scale.

FIG. 1 illustrates a delivery system for a cardiovascular prosthetic device, according to one example.

FIG. 2 illustrates an expandable sheath that can be used in combination with the delivery system of FIG. 1, according to one example.

FIG. 3 is a magnified view of a portion of the expandable sheath of FIG. 2.

FIG. 4 is a side elevation cross-sectional view of a portion of the expandable sheath of FIG. 2.

FIG. 5A is a magnified view of a portion of the expandable sheath of FIG. 2 with the outer layer removed for purposes of illustration.

FIG. 5B is a magnified view of a portion of the braided layer of the sheath of FIG. 2.

FIG. 6 is a magnified view of a portion of the expandable sheath of FIG. 2 illustrating expansion of the sheath as a prosthetic device is advanced through the sheath.

FIG. 7 is a magnified, partial cross-sectional view illustrating the constituent layers of the sheath of FIG. 2 disposed on a mandrel.

FIG. 8 is a magnified view illustrating another example of an expandable sheath.

FIG. 9 is a cross-sectional view of an apparatus that can be used to form an expandable sheath, according to one example.

FIGS. 10A-10D illustrate another example of a braided layer in which the filaments of the braided layer are configured to buckle when the sheath is in a radially collapsed state.

FIG. 11 shows a side cross-sectional view of an assembly of an expandable sheath with a vessel dilator.

FIG. 12 shows the vessel dilator of the assembly example of FIG. 11.

FIG. 13 shows a side view of another assembly example including an expandable sheath and a vessel dilator.

FIG. 14 shows a side view of the assembly example of FIG. 13, with the vessel dilator pushed partially away from the expandable sheath.

FIG. 15 shows a side view of the assembly example of FIG. 13, with the vessel dilator pushed fully away from the expandable sheath.

FIG. 16 shows a side view of the assembly example of FIG. 13, with the vessel dilator being retracted into the expandable sheath.

FIG. 17 shows a side view of the assembly example of FIG. 13, with the vessel dilator being retracted further into the expandable sheath.

FIG. 18 shows a side view of the assembly example of FIG. 13, with the vessel dilator being fully retracted into the expandable sheath.

FIG. 19 shows a side cross sectional view of another assembly example including an expandable sheath and a vessel dilator.

FIG. 20 illustrates and example of a vessel dilator that may be used in combination with the expandable sheaths described herein.

FIG. 21 illustrates an example of a vessel dilator that may be used in combination with the expandable sheaths described herein.

FIG. 22 shows a side view with a cutaway to cross section of an example of an expandable sheath having an outer cover and an overhang.

FIG. 23 shows an example of an outer cover having longitudinal scorelines.

FIG. 24 illustrates an end portion of an example of a braided layer of an expandable sheath.

FIG. 25A illustrates a perspective view of a roller-based crimping mechanism example for crimping an expandable sheath.

FIG. 25B illustrates a side view of a disc-shaped roller and connector of the crimping mechanism shown in FIG. 25A.

FIG. 25C illustrates a top view of a disc-shaped roller and connector of the crimping mechanism shown in FIG. 25A.

FIG. 26 shows an example of a device for crimping an elongated expandable sheath. The encircled portion of the device is magnified in the inset at the left side of the picture.

FIG. 27 shows an example of an expandable sheath having an inner layer with scorelines.

FIG. 28 shows an additional example of a braided layer of an expandable sheath.

FIG. 29 shows a perspective view of an additional expandable sheath example.

FIG. 30 shows a perspective view of the example of FIG. 29 with the outer heat shrink tubing layer partially torn away from the inner sheath layers.

FIG. 31 shows a side view of a sheath example prior to movement of a delivery system therethrough.

FIG. 32 shows a side view of a sheath example as a delivery system moves through, splitting the heat shrink tubing layer.

FIG. 33 shows a side view of a sheath example with the delivery system fully moved through, the heat shrink tubing layer fully split along the length of the sheath.

FIG. 34 shows a perspective view of a sheath example having a distal end portion folded around an introducer.

FIG. 35 shows an enlarged, cross sectional view of the distal end portion folded around the introducer.

FIG. 36 shows a cross section of an additional expandable sheath example.

FIG. 37 shows an example of a cushioning layer.

FIG. 38 shows another example of a cushioning layer.

FIG. 39 shows a side view of an additional expandable sheath example.

FIG. 40 shows a longitudinal cross section of the example of FIG. 39.

FIG. 41 shows a transverse cross section of an additional expandable sheath example.

FIG. 42 shows a partial longitudinal cross section of an additional expandable sheath example.

FIG. 43 shows a transverse cross section of an additional expandable sheath example in an expanded state.

FIG. 44 shows a transverse cross section of the expandable sheath example of FIG. 43 during the crimping process.

FIG. 45 shows a perspective view of a sheath example similar to the sheath of FIG. 43, in the expanded state.

FIG. 46 shows a perspective view of a sheath example similar to the sheath of FIG. 43, in the folded and compressed state.

FIG. 47 shows an additional example of a braided layer.

FIG. 48 is an alternate cross section view of the sheath of FIG. 2 in an unexpanded configuration.

FIG. 49 is an alternate cross section view of the sheath of FIG. 2 in an expanded configuration.

FIG. 50 illustrates an expandable sheath in combination with an expandable introducer, according to one example.

FIG. 51 is a magnified view of a portion of the expandable sheath and introducer of FIG. 50.

FIG. 52 is a magnified view of a portion of the expandable sheath and introducer of FIG. 50.

FIGS. 53A-53J show a magnified view of a portion of the expandable introducer of FIG. 50 with various balloon examples.

FIG. 54 is magnified, partial cross-sectional view illustrating the expandable introducer of FIG. 50.

FIG. 55 is magnified, partial cross-sectional view illustrating the expandable introducer of FIG. 50.

FIG. 56 is magnified, partial cross-sectional view illustrating the expandable introducer of FIG. 50.

FIG. 57 illustrates a side view of an example delivery system for a cardiovascular prosthetic device. is magnified, partial cross-sectional view illustrating the expandable introducer of FIG. 50.

FIG. 58 illustrates a side view of an expansion device in the form of an introducer.

FIG. 59 illustrates a side view of another example of an expansion device in the form for an introducer.

FIG. 60 illustrates another example of an expansion device in the form of an introducer and a corresponding expandable sheath.

FIG. 61 illustrates yet another example of an expansion device in the form of an introducer and a corresponding expandable sheath.

FIG. 62 illustrates a side view of an expansion device in the form of a dilator.

FIG. 63 illustrates a side view of another expansion device in the form of a dilator.

FIG. 64 illustrates an example of an expansion device in the form of an dilator and a corresponding expandable sheath.

Throughout the drawings, it should be noted that like reference numbers are used to depict the same or similar elements, features, and structures.

### DETAILED DESCRIPTION

The expandable introducer sheaths described herein can be used to deliver a prosthetic device through a patient's vasculature to a procedure site within the body. The sheath can be constructed to be highly expandable and collapsible in the radial direction while limiting axial elongation of the sheath and, thereby, undesirable narrowing of the lumen. In one example, the expandable sheath includes a braided layer, one or more relatively thin, non-elastic polymeric layers, and an elastic layer. The sheath can resiliently expand from its natural diameter to an expanded diameter as a prosthetic device is advanced through the sheath, and can return to its natural diameter upon passage of the prosthetic device under the influence of the elastic layer. In certain examples, the one or more polymeric layers can engage the braided layer, and can be configured to allow radial expansion of the braided layer while preventing axial elongation of the braided layer, which would otherwise result in elongation and narrowing of the sheath.

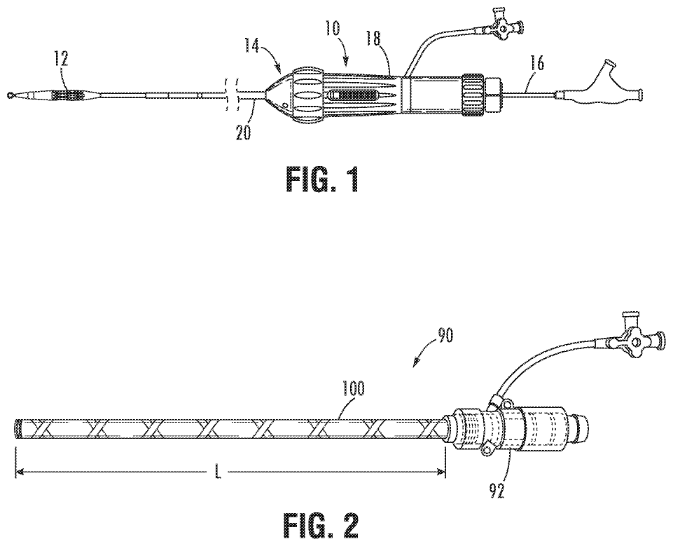

FIG. 1 illustrates a representative delivery apparatus 10 for delivering a medical device, such as a prosthetic heart valve or other prosthetic implant, to a patient. The delivery apparatus 10 is exemplary only, and can be used in combination with any of the expandable sheath examples described herein. Likewise, the sheaths disclosed herein can be used in combination with any of various known delivery apparatuses. The delivery apparatus 10 illustrated can generally include a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate delivery and positioning of a prosthetic heart valve 12 at an implantation site in a patient's body. The guide catheter 14 includes a handle portion 18 and an elongated guide tube or shaft 20 extending from the handle portion 18.

The prosthetic heart valve 12 can be delivered into a patient's body in a radially compressed configuration and radially expanded to a radially expanded configuration at the desired deployment site. In the illustrated example, the prosthetic heart valve 12 is a plastically expandable prosthetic valve that is delivered into the patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Further details regarding a plastically expandable heart valve that can be implanted using the devices disclosed herein are disclosed in U.S. Publication No. 2012/0123529, which is incorporated herein by reference. In other examples, the prosthetic heart valve 12 can be a self-expandable heart valve that is restrained in a radially compressed configuration by a sheath or other component of the delivery apparatus and self-expands to a radially expanded configuration when released by the sheath or other component of the delivery apparatus. Further details regarding a self-expandable heart valve that can be implanted using the devices disclosed herein are disclosed in U.S. Publication No. 2012/0239142, which is incorporated herein by reference. In still other examples, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve. Further details regarding a mechanically expandable heart valve that can be implanted using the devices disclosed herein are disclosed in U.S. Publication No. 2018/0153689, which is incorporated herein by reference. In still other examples, a prosthetic valve can incorporate two or more of the above-described technologies. For example, a self-expandable heart valve can be used in combination with an expansion device to assist expansion of the prosthetic heart valve.

FIG. 2 illustrates an assembly 90 (which can be referred to as an introducer device or assembly) that can be used to introduce the delivery apparatus 10 and the prosthetic device 12 into a patient's body, according to one example. The introducer device 90 can comprise a housing 92 at a proximal end of the device and an expandable sheath 100 extending distally from the housing 92. The housing 92 can function as a handle for the device. The expandable sheath 100 has a central lumen 112 (FIG. 4) to guide passage of the delivery apparatus for the prosthetic heart valve. Generally, during use a distal end of the sheath 100 is passed through the skin of the patient and is inserted into a vessel, such as the femoral artery. The delivery apparatus 10 with its implant 12 can then be inserted through the housing 92 and the sheath 100, and advanced through the patient's vasculature to the treatment site, where the implant is to be delivered and implanted within the patient. In certain examples, the introducer housing 92 can include a hemostasis valve that forms a seal around the outer surface of the guide catheter 14 once inserted through the housing to prevent leakage of pressurized blood.

In alternative examples, the introducer device 90 need not include a housing 92. For example, the sheath 100 can be an integral part of a component of the delivery apparatus 10, such as the guide catheter. For example, the sheath can extend from the handle 18 of the guide catheter. Various example sheaths are described herein. Like reference numbers and designations in the various drawings indicate like elements. Additional examples of introducer devices and expandable sheaths can be found in U.S. patent application Ser. No. 16/378,417, and U.S. Provisional Patent Application No. 62/912,569, filed Oct. 8, 2019 (also filed under International Application No. PCT/US2020/054594), which are incorporated by reference in their entirety.

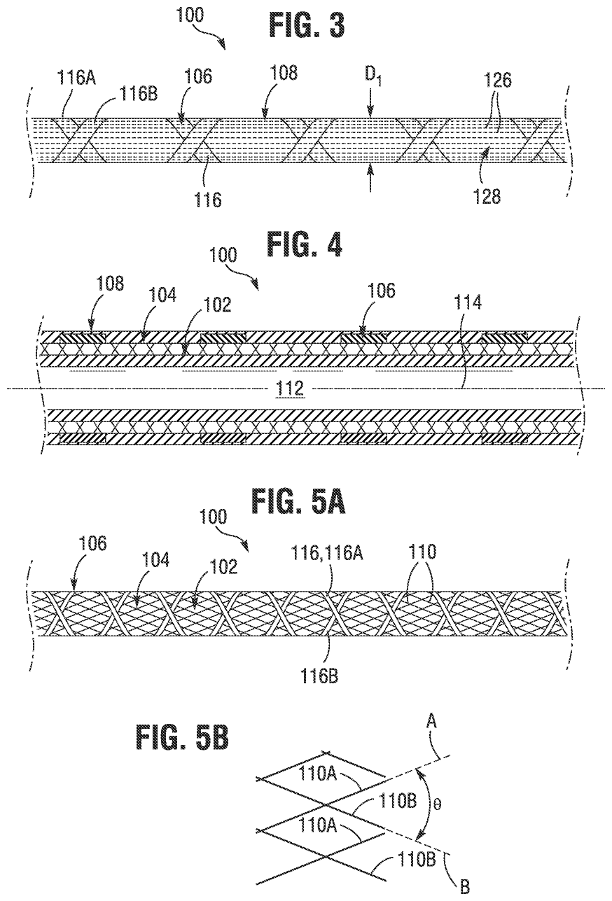

FIG. 3 illustrates the expandable sheath 100 in greater detail. With reference to FIG. 3, the sheath 100 can have a natural, unexpanded outer diameter D₁. In certain examples, the expandable sheath 100 can comprise a plurality of coaxial layers extending along at least a portion of the length L of the sheath (FIG. 2). For example, with reference to FIG. 4, the expandable sheath 100 can include a first layer 102 (also referred to as an inner layer), a second layer 104 disposed around and radially outward of the first layer 102, a third layer 106 disposed around and radially outward of the second layer 104, and a fourth layer 108 (also referred to as an outer layer) disposed around and radially outward of the third layer 106. In the illustrated configuration, the inner layer 102 can define the lumen 112 of the sheath extending along a central axis 114.

Referring to FIG. 3, when the sheath 100 is in an unexpanded state, the inner layer 102 and/or the outer layer 108 can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D₁, the ridges 126 and the valleys 128 can level out or be taken up as the surface radially expands and the circumference increases, as further described below. When the sheath 100 collapses back to its natural diameter, the ridges 126 and valleys 128 can reform.

In certain examples, the inner layer 102 and/or the outer layer 108 can comprise a relatively thin layer of polymeric material. For example, in some sheaths 100 the thickness of the inner layer 102 can be from 0.01 mm to 0.5 mm, 0.02 mm to 0.4 mm, or 0.03 mm to 0.25 mm. In certain examples, the thickness of the outer layer 108 can be from 0.01 mm to 0.5 mm, 0.02 mm to 0.4 mm, or 0.03 mm to 0.25 mm.

In certain examples, the inner layer 102 and/or the outer layer 108 can comprise a lubricious, low-friction, and/or relatively non-elastic material. In particular examples, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (e.g., Pebax), and/or combinations of any of the above. Some examples of a sheath 100 can include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some examples of the sheath 100 can include an exterior hydrophilic coating on the outer surface of the outer layer 108. Such a hydrophilic coating can facilitate insertion of the sheath 100 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidine fluoride), are also suitable for use with the sheath 100. Such hydrophilic coatings may also be included on the inner surface of the inner layer 102 to reduce friction between the sheath and the delivery system, thereby facilitating use and improving safety. In some examples, a hydrophobic coating, such as Perylene, may be used on the outer surface of the outer layer 108 or the inner surface of the inner layer 102 in order to reduce friction.

In certain examples, the second layer 104 can be a braided layer. FIGS. 5A and 5B illustrate the sheath 100 with the outer layer 108 removed to expose the elastic layer 106. With reference to FIGS. 5A and 5B, the braided layer 104 can comprise a plurality of members or filaments 110 (e.g., metallic or synthetic wires or fibers) braided together. The braided layer 104 can have any desired number of filaments 110, which can be oriented and braided together along any suitable number of axes. For example, with reference to FIG. 5B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In certain examples, the angle θ can be from 5° to 70°, 10° to 60°, 10° to 50°, or 10° to 45°. In the illustrated example, the angle θ is 45°. In other examples, the filaments 110 can also be oriented along three axes and braided in a triaxial braid, or oriented along any number of axes and braided in any suitable braid pattern.

The braided layer 104 can extend along substantially the entire length L of the sheath 100, or alternatively, can extend only along a portion of the length of the sheath. In particular examples, the filaments 110 can be wires made from metal (e.g., Nitinol, stainless steel, etc.), or any of various polymers or polymer composite materials, such as carbon fiber. In certain examples, the filaments 110 can be round, and can have a diameter of from 0.01 mm to 0.5 mm, 0.03 mm to 0.4 mm, or 0.05 mm to 0.25 mm. In other examples, the filaments 110 can have a flat cross-section with dimensions of 0.01 mm×0.01 mm to 0.5 mm×0.5 mm, or 0.05 mm×0.05 mm to 0.25 mm×0.25 mm. In one example, filaments 110 having a flat cross-section can have dimensions of 0.1 mm×0.2 mm. However, other geometries and sizes are also suitable for certain examples. If braided wire is used, the braid density can be varied. Some examples have a braid density of from ten picks per inch to eighty picks per inch, and can include eight wires, sixteen wires, or up to fifty-two wires in various braid patterns. In other examples, the second layer 104 can be laser cut from a tube, or laser-cut, stamped, punched, etc., from sheet stock and rolled into a tubular configuration. The layer 104 can also be woven or knitted, as desired.

The third layer 106 can be a resilient, elastic layer (also referred to as an elastic material layer). In certain examples, the elastic layer 106 can be configured to apply force to the underlying layers 102 and 104 in a radial direction (e.g., toward the central axis 114 of the sheath) when the sheath expands beyond its natural diameter by passage of the delivery apparatus through the sheath. Stated differently, the elastic layer 106 can be configured to apply encircling pressure to the layers of the sheath beneath the elastic layer 106 to counteract expansion of the sheath. The radially inwardly directed force is sufficient to cause the sheath to collapse radially back to its unexpanded state after the delivery apparatus is passed through the sheath.

In the illustrated examples, the elastic layer 106 can comprise one or more members configured as strands, ribbons, or bands 116 helically wrapped around the braided layer 104. For example, in the illustrated example the elastic layer 106 comprises two elastic bands 116A and 116B wrapped around the braided layer with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some examples, the elastic layer can comprise an elastomeric material having a modulus of elasticity of 200 MPa or less. In some examples, the elastic layer 106 can comprise a material exhibiting an elongation to break of 200% or greater, or an elongation to break of 400% or greater. The elastic layer 106 can also take other forms, such as a tubular layer comprising an elastomeric material, a mesh, a shrinkable polymer layer such as a heat-shrink tubing layer, etc. In lieu of, or in addition to, the elastic layer 106, the sheath 100 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other examples, the elastic layer 106 can also be radially outward of the polymeric layer 108.

In certain examples, one or both of the inner layer 102 and/or the outer layer 108 can be configured to resist axial elongation of the sheath 100 when the sheath expands. More particularly, one or both of the inner layer 102 and/or the outer layer 108 can resist stretching against longitudinal forces caused by friction between a prosthetic device and the inner surface of the sheath such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 5B, the filaments 110A and 110B of the braided layer can be allowed to move angularly relative to each other such that the angle θ changes as the sheath expands and contracts. This, in combination with the longitudinal folds 126 in the layers 102 and 108, can allow the lumen 112 of the sheath to expand as a prosthetic device is advanced through it.

For example, in some sheaths 100 the inner layer 102 and the outer layer 108 can be heat-bonded during the manufacturing process such that the braided layer 104 and the elastic layer 106 are encapsulated between the layers 102 and 108. More specifically, in certain examples the inner layer 102 and the outer layer 108 can be adhered to each other through the spaces between the filaments 110 of the braided layer 104 and/or the spaces between the elastic bands 116. The layers 102 and 108 can also be bonded or adhered together at the proximal and/or distal ends of the sheath. In certain examples, the layers 102 and 108 are not adhered to the filaments 110. This can allow the filaments 110 to move angularly relative to each other, and relative to the layers 102 and 108, allowing the diameter of the braided layer 104, and thereby the diameter of the sheath, to increase or decrease. As the angle θ between the filaments 110A and 110B changes, the length of the braided layer 104 can also change. For example, as the angle θ increases, the braided layer 104 can foreshorten, and as the angle θ decreases, the braided layer 104 can lengthen to the extent permitted by the areas where the layers 102 and 108 are bonded. However, because the braided layer 104 is not adhered to the layers 102 and 108, the change in length of the braided layer that accompanies a change in the angle θ between the filaments 110A and 110B does not result in a significant change in the length L of the sheath.

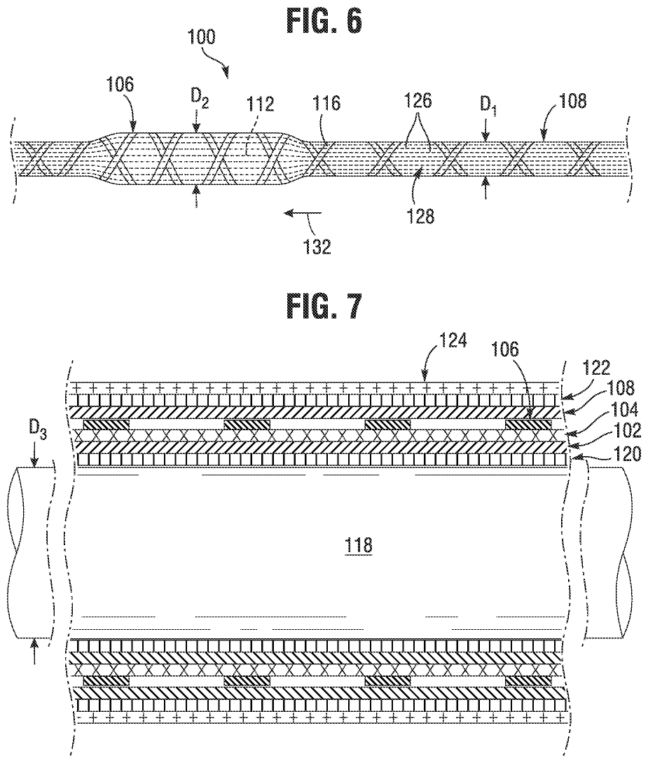

FIG. 6 illustrates radial expansion of the sheath 100 as a prosthetic device 12 is passed through the sheath in the direction of arrow 132 (e.g., distally). As the prosthetic device 12 is advanced through the sheath 100, the sheath can resiliently expand to a second diameter D₂ that corresponds to a size or diameter of the prosthetic device. As the prosthetic device 12 is advanced through the sheath 100, the prosthetic device can apply longitudinal force to the sheath in the direction of motion by virtue of the frictional contact between the prosthetic device and the inner surface of the sheath. However, as noted above, the inner layer 102 and/or the outer layer 108 can resist axial elongation such that the length L of the sheath remains constant, or substantially constant. This can reduce or prevent the braided layer 104 from lengthening, and thereby constricting the lumen 112.

Meanwhile, the angle θ between the filaments 110A and 110B can increase as the sheath expands to the second diameter D₂ to accommodate the prosthetic valve. This can cause the braided layer 104 to foreshorten. However, because the filaments 110 are not engaged or adhered to the layers 102 or 108, the shortening of the braided layer 104 attendant to an increase in the angle θ does not affect the overall length L of the sheath. Moreover, because of the longitudinally-extending folds 126 formed in the layers 102 and 108, the layers 102 and 108 can expand to the second diameter D₂ without rupturing, in spite of being relatively thin and relatively non-elastic. In this manner, the sheath 100 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

Additionally, because of the radial force applied by the elastic layer 106, the radial expansion of the sheath 100 can be localized to the specific portion of the sheath occupied by the prosthetic device. For example, with reference to FIG. 6, as the prosthetic device 12 moves distally through the sheath 100, the portion of the sheath immediately proximal to the prosthetic device 12 can radially collapse back to the initial diameter D₁ under the influence of the elastic layer 106. The layers 102 and 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

In addition to the advantages above, the expandable sheath examples described herein can provide surprisingly superior performance relative to known introducer sheaths. For example, it is possible to use a sheath configured as described herein to deliver a prosthetic device having a diameter that is two times larger, 2.5 times larger, or even three times larger than the natural outer diameter of the sheath. For example, in one example a crimped prosthetic heart valve having a diameter of 7.2 mm was successfully advanced through a sheath configured as described above and having a natural outer diameter of 3.7 mm. As the prosthetic valve was advanced through the sheath, the outer diameter of the portion of the sheath occupied by the prosthetic valve increased to 8 mm. In other words, it was possible to advance a prosthetic device having a diameter more than two times the outer diameter of the sheath through the sheath, during which the outer diameter of the sheath resiliently increased by 216%. In another example, a sheath with an initial or natural outer diameter of 4.5 mm to 5 mm can be configured to expand to an outer diameter of 8 mm to 9 mm.

In alternative examples, the sheath 100 may optionally include the layer 102 without the layer 108, or the layer 108 without the layer 102, depending upon the particular characteristics desired.

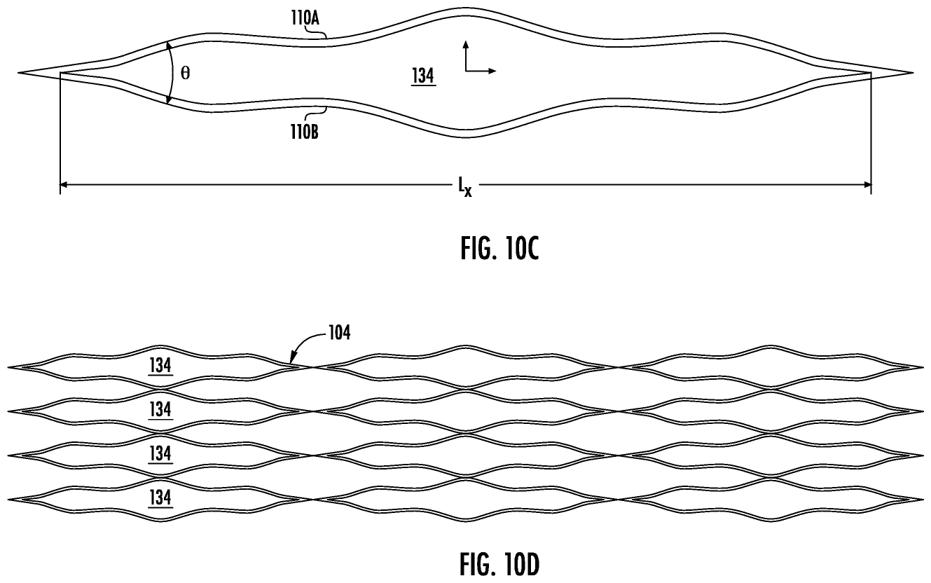

FIGS. 10A-10D illustrate another example of the braided layer 104 in which the filaments 110 are configured to buckle. For example, FIG. 10A illustrates a unit cell 134 of the braided layer 104 in a configuration corresponding to the braided layer in a fully expanded state. For example, the expanded state illustrated in FIG. 10A can correspond to the diameter D₂ described above, and/or a diameter of the braided layer during initial construction of the sheath 100 before the sheath is radially collapsed to its functional design diameter D₁, as described further below with reference to FIG. 7. The angle θ between the filaments 110A and 110B can be, for example, 40°, and the unit cell 134 can have a length Lₓ along the x-direction (note Cartesian coordinate axes shown). FIG. 10B illustrates a portion of the braided layer 104 including an array of unit cells 134 in the expanded state.

In the illustrated examples, the braided layer 104 is disposed between the polymeric layers 102 and 108, as described above. For example, the polymeric layers 102 and 108 can be adhered or laminated to each other at the ends of the sheath 100 and/or between the filaments 110 in the open spaces 136 defined by the unit cells 134. Thus, with reference to FIGS. 10C and 10D, when the sheath 100 is radially collapsed to its functional diameter D₁, the diameter of the braided layer 104 can decrease as the angle θ decreases. However, the bonded polymeric layers 102 and 108 can constrain or prevent the braided layer 104 from lengthening as it radially collapses. This can cause the filaments 110 to resiliently buckle in the axial direction, as shown in FIGS. 10C and 10D. The degree of buckling can be such that the length Lₓ of the unit cells 134 is the same, or substantially the same, between the collapsed and fully expanded diameters of the sheath. This means that the overall length of the braided layer 104 can remain constant, or substantially constant, between the natural diameter D₁ of the sheath and the expanded diameter D₂. As the sheath expands from in its initial diameter D₁ during passage of a medical device, the filaments 110 can straighten as the buckling is relieved, and the sheath can radially expand. As the medical device passes through the sheath 100, the braided layer 104 can be urged back to the initial diameter D₁ by the elastic layer 106, and the filaments 110 can resiliently buckle again. Using the configuration of FIGS. 10A-10C, it is also possible to accommodate a prosthetic device having a diameter that is two times larger, 2.5 times larger, or even three times larger than the natural outer diameter D₁ of the sheath.

Turning now to methods of making expandable sheaths, FIG. 7 illustrates the layers 102-108 of the expandable sheath 100 disposed on a cylindrical mandrel 118, according to one example. In certain examples, the mandrel 118 can have a diameter D₃ that is greater than the desired natural outer diameter D₁ of the finished sheath. For example, in some examples a ratio of the diameter D₃ of the mandrel to the outer diameter D₁ of the sheath can be 1.5:1, 2:1, 2.5:1, 3:1, or greater. In certain examples, the diameter D₃ of the mandrel can be equal to the expanded diameter D₂ of the sheath. In other words, the diameter D₃ of the mandrel can be the same, or nearly the same, as the desired expanded diameter D₂ of the sheath when a prosthetic device is being advanced through the sheath. Thus, in certain examples a ratio of the expanded outer diameter D₂ of the expanded sheath to the collapsed outer diameter D₁ of the unexpanded sheath can be 1.5:1, 2:1, 2.5:1, 3:1, or greater.

With reference to FIG. 7, the expandable sheath 100 can be made by wrapping or situating an ePTFE layer 120 around the mandrel 118, followed by the first polymeric layer 102. In some examples, the ePTFE layer can aid in removing the sheath 100 from the mandrel 118 upon completion of the fabrication process. The first polymeric layer 102 may be in the form of a pre-fabricated sheet that is applied by being wrapped around the mandrel 118, or may be applied to the mandrel by dip-coating, electro-spinning, etc. The braided layer 104 can be situated around the first layer 102, followed by the elastic layer 106. In examples in which the elastic layer 106 comprises one or more elastic bands 116, the bands 116 can be helically wrapped around the braided layer 104. In other examples, the elastic layer 106 may be dip-coated, electro-spun, etc. The outer polymeric layer 108 can then be wrapped, situated, or applied around the elastic layer 106, followed by another layer 122 of ePTFE and one or more layers 124 of heat-shrink tubing or heat-shrink tape.

In particular examples, the elastic bands 116 can be applied to the braided layer 104 in a stretched, taut, or extended condition. For example, in certain examples the bands 116 can be applied to the braided layer 104 stretched to a length that is twice their natural, relaxed length. This will cause the completed sheath to radially collapse under the influence of the elastic layer when removed from the mandrel, which can cause corresponding relaxation of the elastic layer, as described below. In other examples, the layer 102 and the braided layer 104 can be removed from the mandrel, the elastic layer 106 can be applied in a relaxed state or moderately stretched state, and then the assembly can be placed back on the mandrel such that the elastic layer is radially expanded and stretched to a taut condition prior to application of the outer layer 108.

The assembly can then be heated to a sufficiently high temperature that the heat-shrink layer 124 shrinks and compresses the layers 102-108 together. In certain examples, the assembly can be heated to a sufficiently high temperature such that the polymeric inner and outer layers 102 and 108 become soft and tacky, and bond to each other in the open spaces between the braided layer 104 and the elastic layer 106 and encapsulate the braided layer and the elastic layer. In other examples, the inner and outer layers 102, 108 can be reflowed or melted such that they flow around and through the braided layer 104 and the elastic layer 106. In one example, the assembly can be heated at 150° C. for 20-30 minutes.

After heating, the sheath 100 can be removed from the mandrel 118, and the heat-shrink tubing 124 and the ePTFE layers 120 and 122 can be removed. Upon being removed from the mandrel 118, the sheath 100 can at least partially radially collapse to the natural design diameter D₁ under the influence of the elastic layer 106. In certain examples, the sheath can be radially collapsed to the design diameter with the optional aid of a crimping mechanism. The attendant reduction in circumference can buckle the filaments 110 as shown in FIGS. 10C and 10D, along with the inner and outer layers 102 and 108 to form the longitudinally-extending folds 126.

In certain examples, a layer of PTFE can be interposed between the ePTFE layer 120 and the inner layer 102, and/or between the outer layer 108 and the ePTFE layer 122, in order to facilitate separation of the inner and outer polymeric layers 102, 108 from the respective ePTFE layers 120 and 122. In further examples, one of the inner layer 102 or the outer layer 108 may be omitted, as described above.

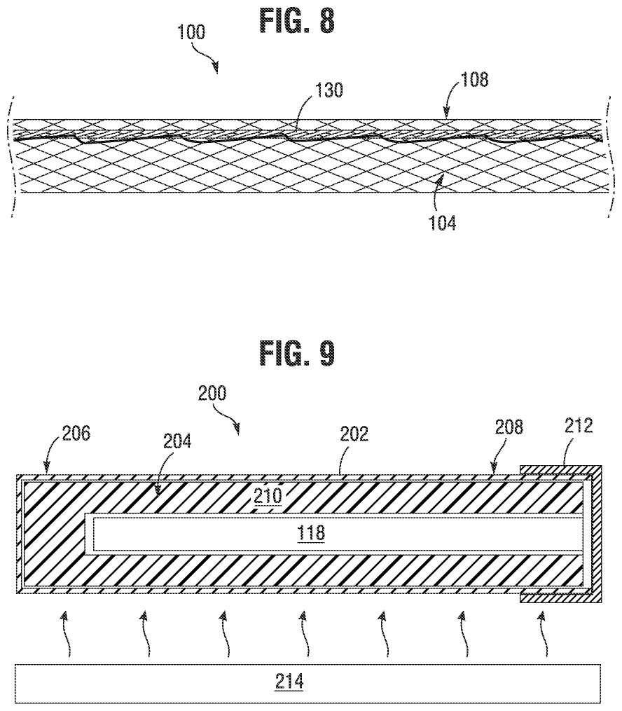

FIG. 8 illustrates another example of the expandable sheath 100 including one or more members configured as yarns or cords 130 extending longitudinally along the sheath and attached to the braided layer 104. Although only one cord 130 is illustrated in FIG. 8, in practice the sheath may include two cords, four cords, six cords, etc., arrayed around the circumference of the sheath at equal angular spacings. The cords 130 can be sutured to the exterior of the braided layer 104, although other configurations and attachment methods are possible. By virtue of being attached to the braided layer 104, the cords 130 can be configured to prevent axial elongation of the braided layer 104 when a prosthetic device is passed through the sheath. The cords 130 may be employed in combination with the elastic layer 106, or separately. The cords 130 may also be used in combination with one or both of the inner and/or outer layers 102 and 108, depending upon the particular characteristics desired. The cords 130 may also be disposed on the inside of the braided layer 104 (e.g., between the inner layer 102 and the braided layer 104).

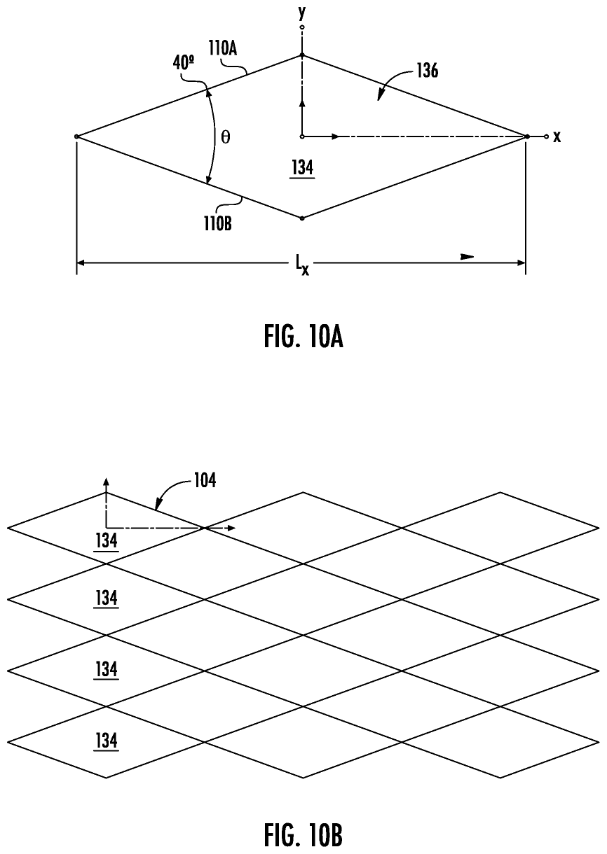

The expandable sheath 100 can also be made in other ways. For example, FIG. 9 illustrates an apparatus 200 including a containment vessel 202 and a heating system schematically illustrated at 214. The apparatus 200 is particularly suited for forming devices (medical devices or devices for non-medical uses) comprised of two or more layers of material. Devices formed by the apparatus 200 can be formed from two or more co-axial layers of material, such as the sheath 100, or shafts for catheters. Devices formed by the apparatus 200 alternatively can be formed by two or more non-coaxial layers, such as two or more layers stacked on top of each other.

The containment vessel 202 can define an interior volume or chamber 204. In the illustrated examples, the vessel 202 can be a metal tube including a closed end 206 and an open end 208. The vessel 202 can be at least partially filled with a thermally-expandable material 210 having a relatively high coefficient of thermal expansion. In particular examples, the thermally-expandable material 210 may have a coefficient of thermal expansion of 2.4×10⁻⁴/° C. or greater. Exemplary thermally-expandable materials include elastomers such as silicones materials. Silicone materials can have a coefficient of thermal expansion of from 5.9×10⁻⁴/° C. to 7.9×10⁻⁴/° C.

A mandrel similar to the mandrel 118 of FIG. 7 and including the desired combination of sheath material layers disposed around it can be inserted into the thermally-expandable material 210. Alternatively, the mandrel 118 can be inserted into the chamber 204, and the remaining volume of the chamber can be filled with the thermally-expandable material 210 so that the mandrel is surrounded by the material 210. The mandrel 118 is shown schematically for purposes of illustration. As such, the mandrel 118 can be cylindrical as depicted in FIG. 7. Likewise, the inner surface of the material 210 and the inner surface of the vessel 202 can have a cylindrical shape that corresponds to the shape of the mandrel 118 and the final shape of the sheath 100. To facilitate placement of a cylindrical or rounded mandrel 118, the vessel 202 can comprise two portions that are connected to each other by a hinge to allow the two portions to move between an open configuration for placing the mandrel inside of the vessel and a closed configuration extending around the mandrel. For example, the upper and lower halves of the vessel shown in FIG. 9 can be connected to each other by a hinge at the closed side of the vessel (the left side of the vessel in FIG. 9).

The open end 208 of the vessel 202 can be closed with a cap 212. The vessel 202 can then be heated by the heating system 214. Heating by the heating system 214 can cause the material 210 to expand within the chamber 204 and apply radial pressure against the layers of material on the mandrel 118. The combination of the heat and pressure can cause the layers on the mandrel 118 to bond or adhere to each other to form a sheath. In certain examples, it is possible to apply radial pressure of 100 MPa or more to the mandrel 118 using the apparatus 200. The amount of radial force applied to the mandrel can be controlled by, for example, the type and quantity of the material 210 selected and its coefficient of thermal expansion, the thickness of the material 210 surrounding the mandrel 118, the temperature to which the material 210 is heated, etc.

In some examples, the heating system 214 can be an oven into which the vessel 202 is placed. In some examples, the heating system can include one or more heating elements positioned around the vessel 202. In some examples, the vessel 202 can be an electrical resistance heating element or an induction heating element controlled by the heating system 214. In some examples, heating elements can be embedded in the thermally-expandable material 210. In some examples, the material 210 can be configured as a heating element by, for example, adding electrically conductive filler materials, such as carbon fibers or metal particles.

The apparatus 200 can provide several advantages over known methods of sheath fabrication, including uniform, highly controllable application of radial force to the mandrel 118 along its length, and high repeatability. The apparatus 200 can also facilitate fast and accurate heating of the thermally-expandable material 210, and can reduce or eliminate the need for heat-shrink tubing and/or tape, reducing material costs and labor. The amount of radial force applied can also be varied along the length of the mandrel by, for example, varying the type or thickness of the surrounding material 210. In certain examples, multiple vessels 202 can be processed in a single fixture, and/or multiple sheaths can be processed within a single vessel 202. The apparatus 200 can also be used to produce other devices, such as shafts or catheters.

In one specific method, the sheath 100 can be formed by placing layers 102, 104, 106, 108 on the mandrel 118 and placing the mandrel with the layers inside of the vessel 202 with the thermally-expandable material 210 surrounding the outermost layer 108. If desired, one or more inner layers 120 of ePTFE (or similar material) and one or more outer layers 122 of ePTFE (or similar material) can be used (as shown in FIG. 7) to facilitate removal of the finished sheath from the mandrel 118 and the material 210. The assembly is then heated with the heating system 214 to reflow the layers 102, 108. Upon subsequent cooling, the layers 102, 108 become at least partially bonded to each other and at least partially encapsulate layers 104, 106.

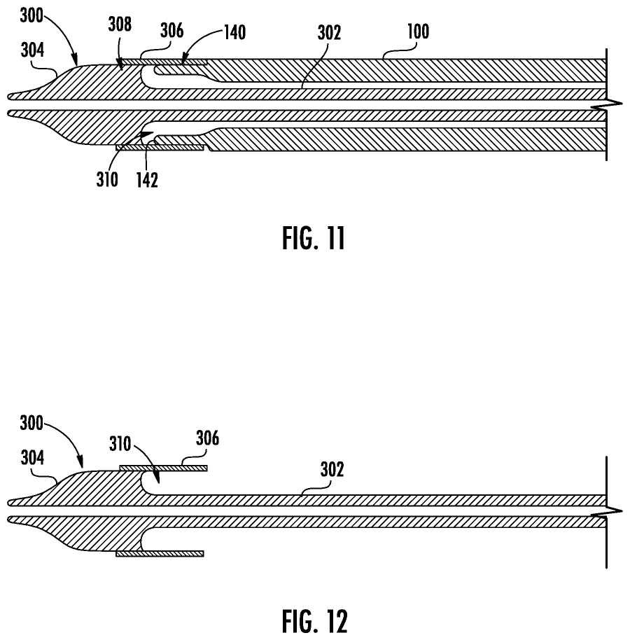

FIG. 11 illustrates another example in which the expandable sheath 100 is configured to receive an apparatus configured as a pre-introducer or vessel dilator 300. In particular examples, the introducer device 90 can include the vessel dilator 300. Referring to FIG. 12, the vessel dilator 300 can comprise a shaft member 302 including a tapered dilator member configured as a nose cone 304 located at the distal end portion of the shaft member 302. The vessel dilator 300 can further comprise a capsule or retaining member 306 extending proximally from a proximal end portion 308 of the nose cone 304 such that a circumferential space 310 is defined between the exterior surface of the shaft member 302 and the interior surface of the retaining member 306. In certain examples, the retaining member 306 can be configured as a thin polymeric layer or sheet, as further described below.

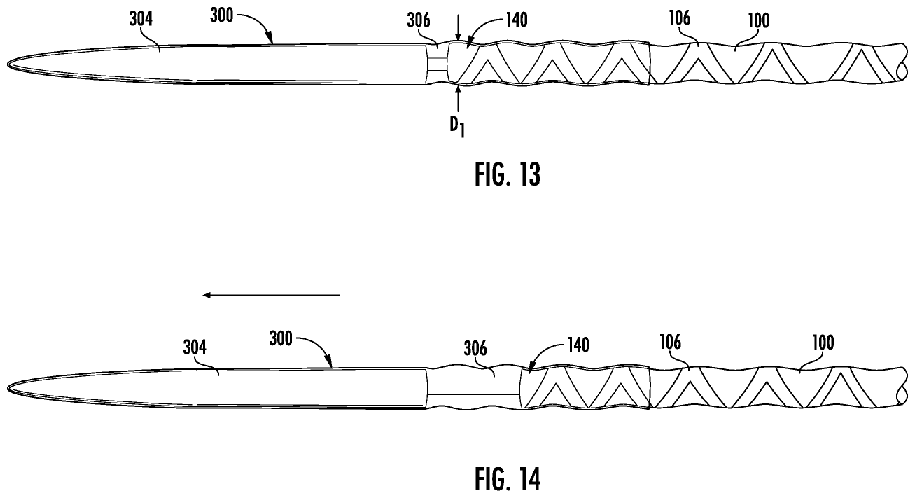

Referring to FIGS. 11 and 13, a first or distal end portion 140 of the sheath 100 can be received in the space 310 such that the sheath engages the nose cone 304, and/or such that the retaining member 306 extends over the distal end portion 140 of the sheath. In use, the coupled or assembled vessel dilator 300 and sheath 100 can then be inserted through an incision into a blood vessel. The tapered cone shape of the nose cone 304 can aid in gradually dilating the blood vessel and access site while minimizing trauma to the blood vessel and surrounding tissue. Once the assembly has been inserted to the desired depth, the vessel dilator 300 can be advanced further into the blood vessel (e.g., distally) while the sheath 100 is held steady, as illustrated in FIG. 14.

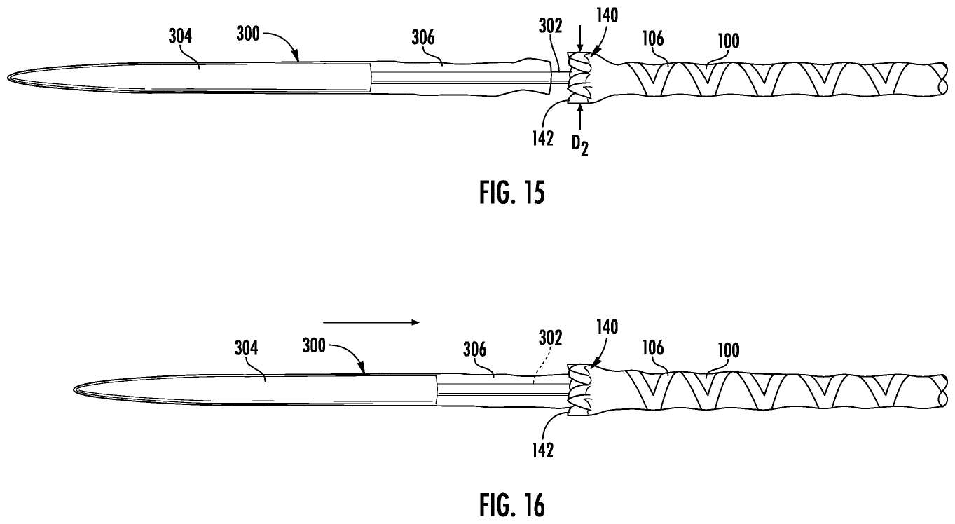

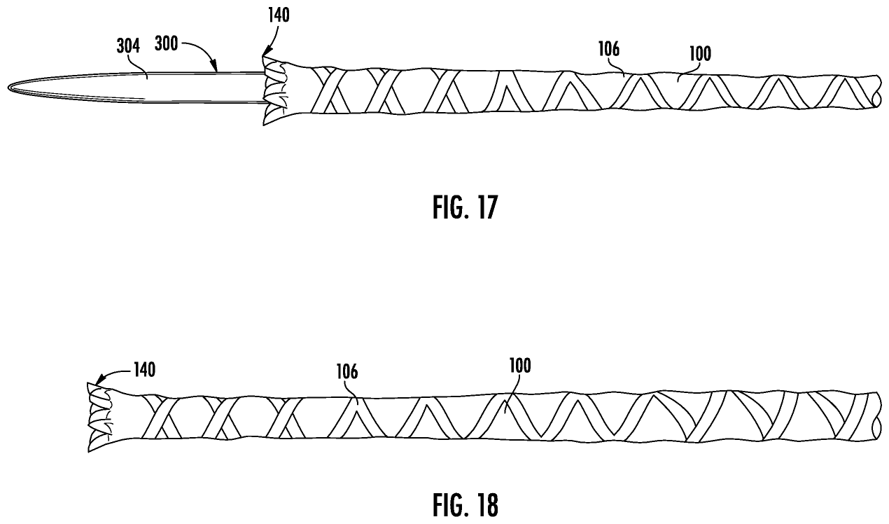

Referring to FIG. 15, the vessel dilator 300 can be advanced distally through the sheath 100 until the retaining member 306 is removed from over the distal end portion 140 of the sheath 100. In certain examples, the helically-wrapped elastic layer 106 of the sheath can terminate proximally of the distal end 142 of the sheath. Thus, when the distal end portion 140 of the sheath is uncovered, the distal end portion (which can be heat-set) can flare or expand, increasing the diameter of the opening at the distal end 142 from the first diameter D₁ (FIG. 13) to a second, larger diameter D₂ (FIG. 15). The vessel dilator 300 can then be withdrawn through the sheath 100 as illustrated in FIGS. 16-18, leaving the sheath 100 in place in the vessel.

The vessel dilator 300 can include a variety of active and/or passive mechanisms for engaging and retaining the sheath 100. For example, in certain examples the retaining member 306 can comprise a polymeric heat-shrink layer that can be collapsed around the distal end portion of the sheath 100. In the examples illustrated in FIG. 1, the retaining member can comprise an elastic member configured to compress the distal end portion 140 of the sheath 100. In yet other examples, the retaining member 306 and the sheath 100 can be glued or fused (e.g., heat-bonded) together in a manner such that application of selected amount of force can break the adhesive bonds between retaining member 306 free from the sheath 100 to allow the vessel dilator to be withdrawn. In some examples, the end portion of the braided layer 104 can be heat set to flare or expand radially inwardly or outwardly, in order to apply pressure to a corresponding portion of the vessel dilator 300.

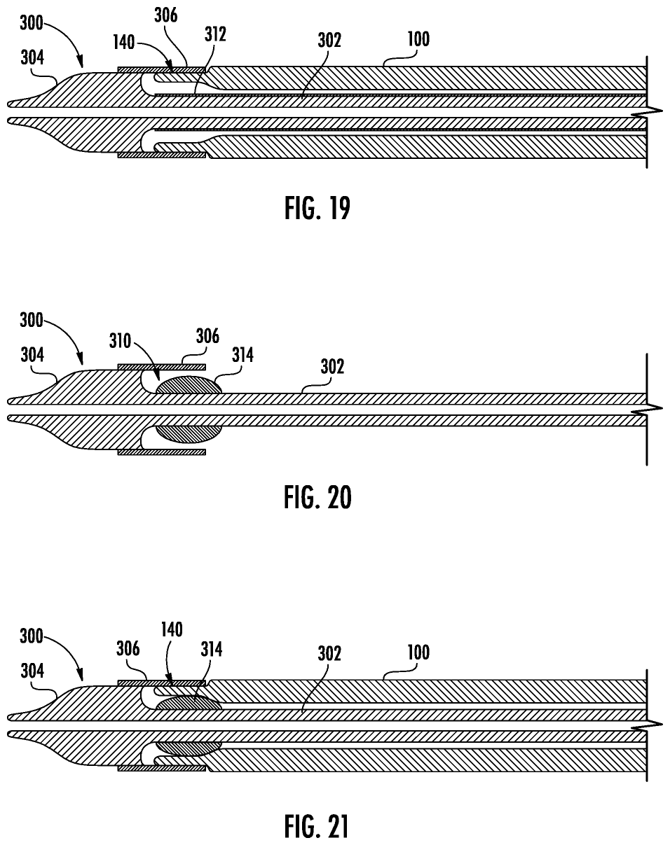

Referring to FIG. 19, the assembly can include a mechanically-actuated retaining mechanism, such as a shaft 312 disposed between the dilator shaft member 302 and the sheath 100. In certain examples, the shaft 312 can releasably couple the vessel dilator 300 to the sheath 100, and can be actuated from outside the body (i.e., manually deactivated).

Referring to FIGS. 20 and 21, in some examples the shaft 304 can comprise one or more balloons 314 arrayed circumferentially around its exterior surface and configured to engage the sheath 100 when inflated. The balloons 314 can be selectively deflated in order to release the sheath 100 and withdraw the vessel dilator. For example, when inflated, the balloons press the captured distal end portion of the sheath 100 against the inner surface of the capsule 306 to assist in retaining the sheath in place relative to the vessel dilator. When the balloons are deflated, the vessel dilator can be more easily moved relative to the sheath 100.

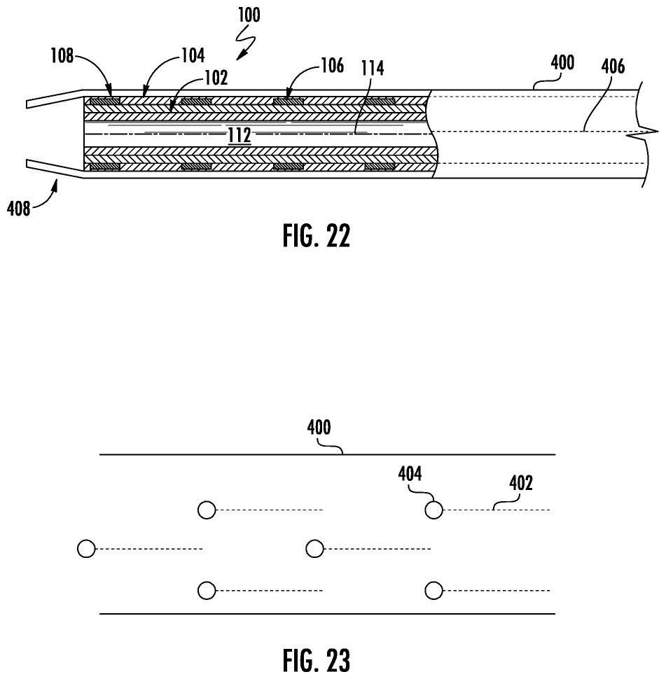

In another examples, an expandable sheath configured as described above can further comprise a shrinkable polymeric outer cover, such as a heat-shrink tubing layer 400 shown in FIG. 22. The heat-shrink tubing layer 400 can be configured to allow a smooth transition between the vessel dilator 300 and the distal end portion 140 of the sheath. The heat-shrink tubing layer 400 can also constrain the sheath to a selected initial, reduced outer diameter. In certain examples, the heat-shrink tubing layer 400 extends fully over the length of the sheath 100 and can be attached to the sheath handle by a mechanical fixation means, such as a clamp, nut, adhesive, heat welding, laser welding, or an elastic clamp. In some examples, the sheath 100 is press-fit into the heat-shrink tubing layer 400 during manufacturing.

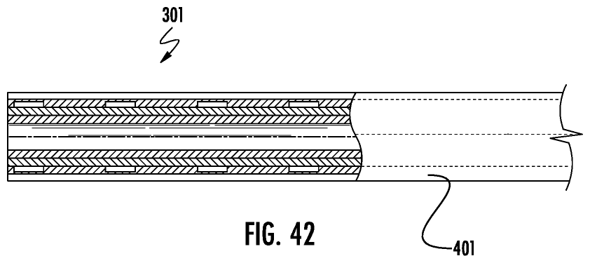

In some examples, the heat-shrink tubing layer 400 can extend distally beyond the distal end portion 140 of the sheath 100 as the distal overhang 408 shown in FIG. 22. A vessel dilator 300 can be inserted through the sheath lumen 112 and beyond the distal edge of the overhang 408. The overhang 408 conforms tightly to the inserted vessel dilator 300 to give a smooth transition between the dilator diameter and the sheath diameter to ease insertion of the combined dilator 300 and sheath 100. When the vessel dilator 300 is removed, overhang 408 remains in the vessel as part of sheath 100. The heat shrink tubing layer 400 offers the additional benefit of shrinking the overall outer diameter of the sheath 100 along the longitudinal axis. However, it will be understood that some examples, such as sheath 301 shown at FIG. 42 may have a heat-shrink tubing layer 401 that stops at the distal end of the sheath 301 or, in some examples, does not extend fully to the distal end of the sheath. In examples without distal overhangs, the heat-shrink tubing layer functions mainly as an outer shrinking layer, configured to maintain the sheath in a compressed configuration. Such examples will not result in a flapping overhang at the distal end of the sheath once the dilator is retrieved.

In some examples, the heat-shrink tubing layer 400 can be configured to split open as a delivery apparatus such as the delivery apparatus 10 is advanced through the sheath 100. For example, the heat-shrink tubing layer 400 can comprise one or more longitudinally extending openings, slits, or weakened, elongated scorelines 406 such as those shown in FIG. 22 configured to initiate splitting of the layer at a selected location. As the delivery apparatus 10 is advanced through the sheath 100, the heat-shrink tubing layer 400 can continue to split open, allowing the sheath to expand as described above with reduced force. In certain examples, the sheath 100 need not comprise the elastic layer 106 such that the sheath automatically expands from the initial, reduce diameter when the heat-shrink tubing layer 400 splits open. The heat shrink tubing layer 400 can comprise polyethylene or other suitable materials.

FIG. 23 illustrates a heat-shrink tubing layer 400 that can be placed around the expandable sheaths described herein, according to one example. In some examples, the heat-shrink tubing layer 400 can comprise a plurality of cuts or scorelines 402 extending axially along the tubing layer 400 and terminating at distal stress relief features configured as circular openings 404. It is contemplated that the distal stress relief feature can be configured as any other regular or irregular curvilinear shape including, for example, oval and/or ovoid shaped openings. It is also contemplated various shaped distal stress relief features along and around the heat-shrink tubing layer 400. As the delivery apparatus 10 is advanced through the sheath 100, the heat-shrink tubing layer 400 can split open along the scorelines 402, and the distally positioned openings 404 can arrest further tearing or splitting of the tubing layer along the respective scorelines 402. As such, the heat-shrink tubing layer 400 remains attached to the sheath 100 along the sheath length. In the illustrated example, the scorelines 402 and associated openings 404 are longitudinally and circumferentially offset from one another or staggered. Thus, as the sheath 100 expands, the scorelines 402 can form rhomboid structures. The scorelines 402 can also extend in other directions, such as helically around the longitudinal axis of the sheath 100, or in a zig-zag pattern

In other examples, splitting or tearing of the heat-shrink tubing layer 400 may be induced in a variety of other ways, such as by forming weakened areas on the tubing surface by, for example, applying chemical solvents, cutting, scoring, or ablating the surface with an instrument or laser, and/or by decreasing the wall thickness or making cavities in the tubing wall (e.g., by femto-second laser ablation).

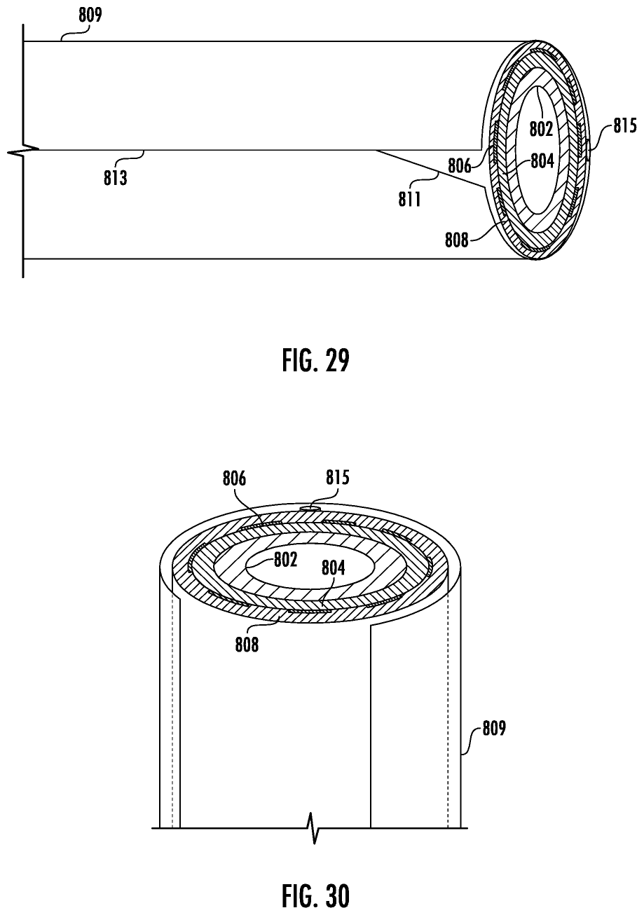

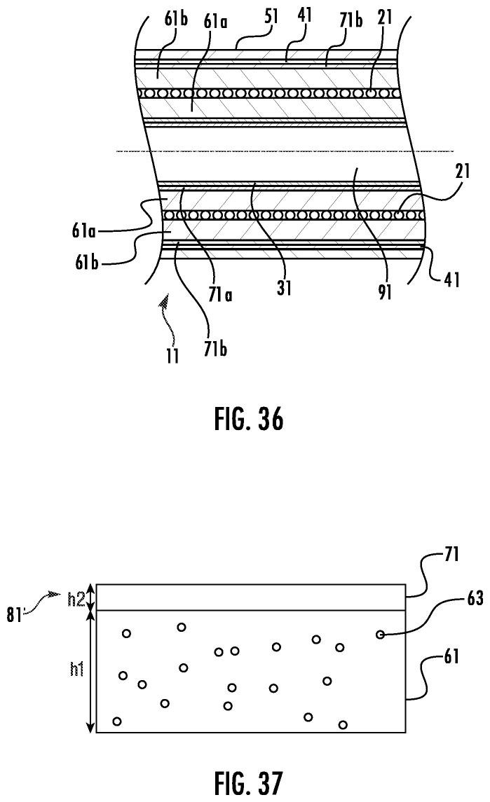

In some examples, the heat-shrink tubing layer may be attached to the body of the sheath by adhesive, welding, or any other suitable fixation means. FIG. 29 shows a perspective view of an example sheath including an inner layer 802, a braided layer 804, an elastic layer 806, an outer layer 808, and a heat shrink tubing layer 809. As described below with respect to FIG. 36, some examples may not include elastic layer 806. Heat shrink tubing layer 809 includes a split 811 and a perforation 813 extending along the heat shrink tubing layer 809. Heat shrink tubing layer 809 is bonded to the outer layer 808 at an adhesive seam 815. For example, in certain examples the heat-shrink tubing layer 809 can be welded, heat-bonded, chemically bonded, ultrasonically bonded, and/or bonded using adhesive agents (including, but not limited to, hot glue, for example LDPE fiber hot glue) at seam 815. The outer layer 808 can be bonded to the heat shrink tubing layer 809 axially along the sheath at a seam 815, or in a spiral or helical fashion. FIG. 30 shows the same example sheath with heat shrink tubing layer 809 split open at the distal end of the sheath.

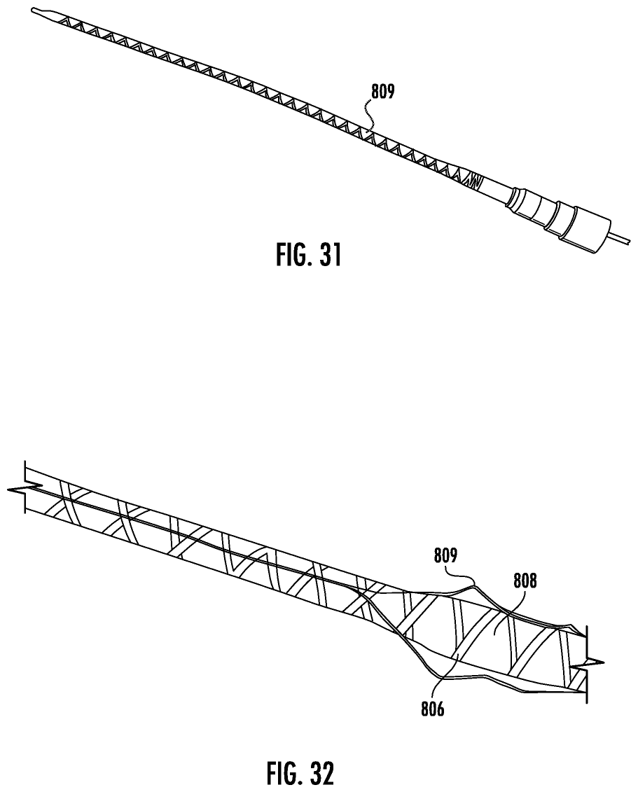

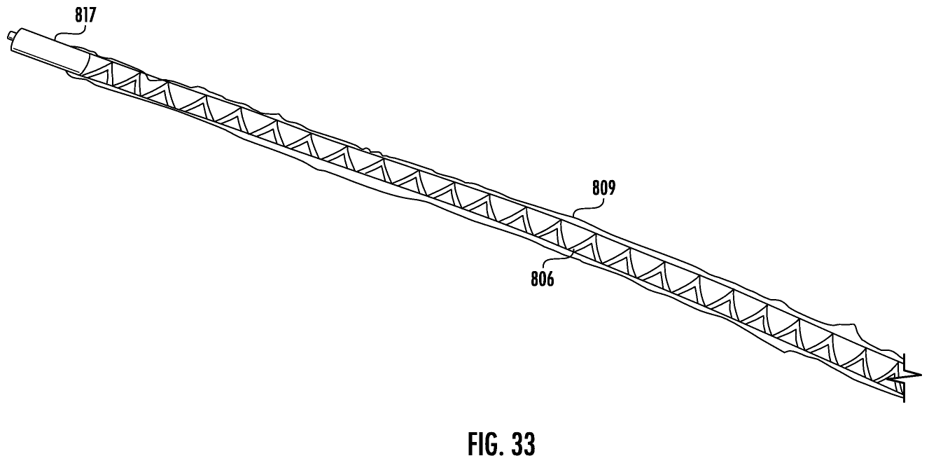

FIG. 31 shows a sheath having a heat shrink tubing layer 809, but prior to movement of a delivery system therethrough. FIG. 32 shows a perspective view of a sheath wherein the heat shrink tubing layer 809 has been partially torn open and detached as a passing delivery system widens the diameter of the sheath. Heat shrink tubing layer 809 is being retained by the adhesive seam 815. Attaching the heat-shrink tubing layer 809 to the sheath in this manner can help to keep the heat-shrink tubing layer 809 attached to the sheath after the layer splits and the sheath has expanded, as shown in FIG. 33, where delivery system 817 has moved completely through the sheath and torn the heat shrink tubing layer 809 along the entire length of the sheath.

In another example, the expandable sheath 100 can have a distal end or tip portion comprising an elastic thermoplastic material (e.g., Pebax), which can be configured to provide an interference fit or interference geometry with the corresponding portion of the vessel dilator 300. In certain configurations, the outer layer of the sheath 100 may comprise polyamide (e.g., nylon) in order to provide for welding the distal end portion to the body of the sheath 100. In certain examples, the distal end portion 140 can comprise a deliberately weakened portion, scoreline, slit, etc., to allow the distal end portion 140 to split apart as the delivery apparatus 10 is advanced through the distal end portion 140.

In another examples, the entire sheath 100 could have an elastomeric outer cover that extends longitudinally from the handle to the distal end portion 140 of the sheath, optionally extending onward to create an overhang similar to overhang 408 shown in FIG. 22. The elastomeric overhang portion conforms tightly to the vessel dilator 300 but remains a part of the sheath once the vessel dilator 300 is removed. As a delivery system is passed through, the elastomeric overhang portion expands and then collapses to allow it to pass. The elastomeric overhang portion, or the entire elastomeric outer cover, can include deliberately weakened portions, scorelines, slits, etc. to allow the distal end portion 140 to split apart as the delivery apparatus is advanced through the distal end portion 140.

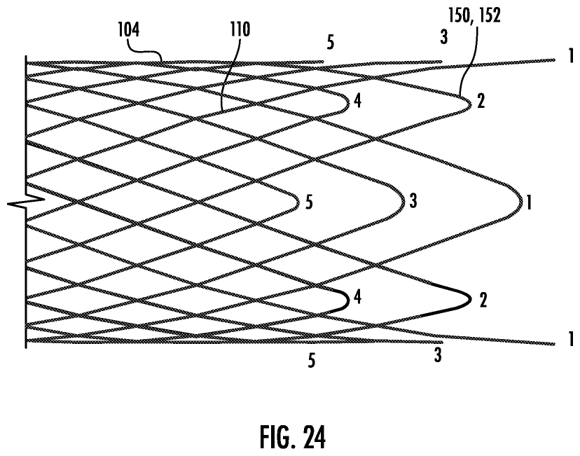

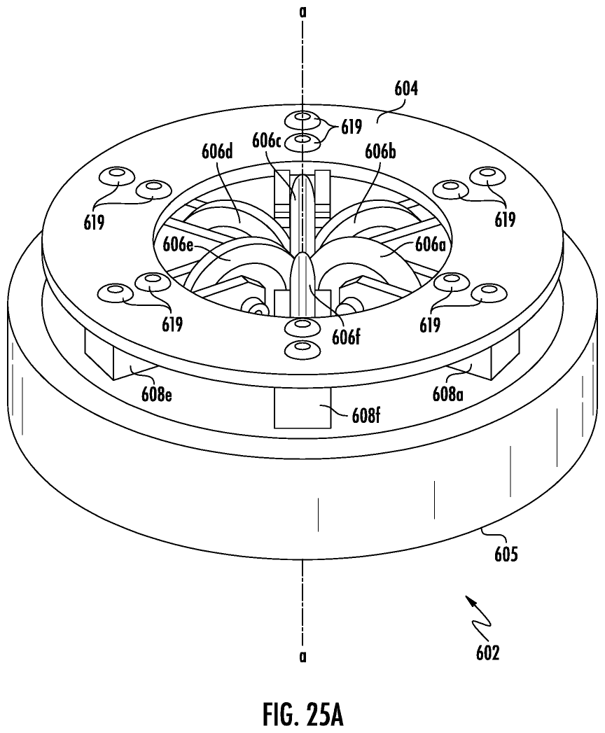

FIG. 24 illustrates an end portion (e.g., a distal end portion) of another example of the braided layer 104 in which portions 150 of the braided filaments 110 are bent to form loops 152, such that the filaments 110 loop or extend back in the opposite direction along the sheath 100. The filaments 110 can be arranged such that the loops 152 of various filaments 110 are axially offset from each other in the braid. Moving toward the distal end of the braided layer 104 (to the right in the figure), the number of braided filaments 110 can decrease. For example, the filaments 110 indicated at 5 can form loops 152 first, followed by the filaments 110 indicated at 4, 3, and 2, with the filaments 110 at 1 forming the distal-most loops 152. Thus, the number of filaments 110 in the braid decreases in the distal direction, which can increase the radial flexibility of the braided layer 104.

In another example, the distal end portion 140 of the expandable sheath 100 can comprise a polymer such as Dyneema®, which can be tapered to the diameter of the vessel dilator 300. Weakened portions such as dashed cuts, scoring, etc., can be applied to the distal end portion 140 such that it will split open and/or expand in a repeatable way.

Crimping of the expandable sheath 100 examples described herein can be performed in a variety of ways, as described above. In additional examples, the sheath 100 can be crimped using a conventional short crimper several times longitudinally along the longer sheath 100. In other examples, the sheath 100 may be collapsed to a specified crimped diameter in one or a series of stages in which the sheath is wrapped in heat-shrink tubing and collapsed under heating. For example, a first heat shrink tube can be applied to the outer surface of the sheath 100, the sheath 100 can be compressed to an intermediate diameter by shrinking the first heat shrink tube (via heat), the first heat shrink tube can be removed, a second heat shrink tube can be applied to the outer surface of the sheath 100, the second heat shrink tube can be compressed via heat to a diameter smaller than the intermediate diameter, and the second heat shrink tube can be removed. This can go on for as many rounds as necessary to achieve the desired crimped sheath diameter.

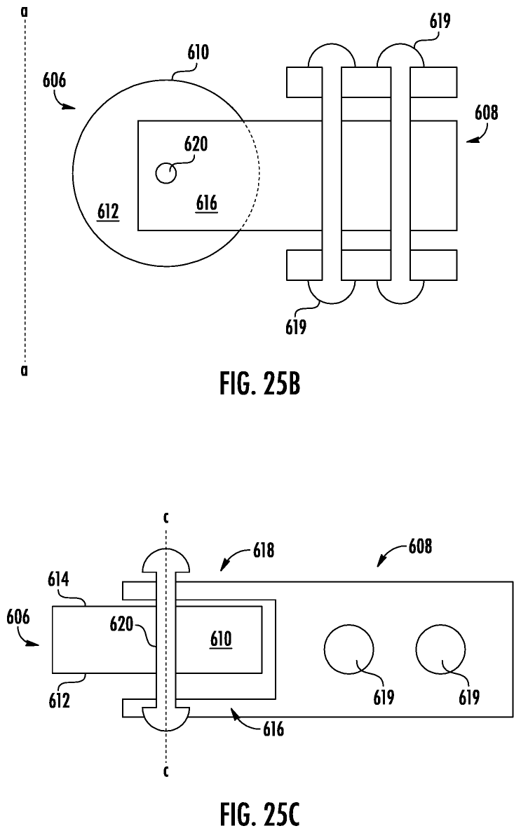

Crimping of the various expandable sheath examples described herein can be performed in a variety of ways, as described above. A roller-based crimping mechanism 602, such as the one shown in FIGS. 25A-25C, can be advantageous for crimping elongated structures such as the sheaths disclosed herein. The crimping mechanism 602 has a first end surface 604, a second end surface 605, and a longitudinal axis a-a extending between the first and second end surfaces 604, 605. A plurality of disc-shaped rollers 606a-f are radially arranged about the longitudinal axis a-a, each positioned at least partially between the first and second end surfaces of the crimping mechanism 602. Six rollers are depicted in the example shown, but the number of rollers may vary. Each disc-shaped roller 606 is attached to the larger crimping mechanism by a connector 608. A side cross-sectional view of an individual disc-shaped roller 606 and connector 608 is shown in FIG. 25B, and a top view of an individual disc-shaped roller 606 and connector 608 is shown in FIG. 25C. An individual disc-shaped roller 606 has a circular edge 610, a first side surface 612, a second side surface 614, and a central axis c-c extending between center points of first and second side surfaces 612, 614, as shown in FIG. 25C. The plurality of disc-shaped rollers 606a-f are radially arranged about the longitudinal axis a-a of the crimping mechanism 602 such that each central axis c-c of a disc-shaped roller 606 is oriented perpendicularly to the longitudinal axis a-a of the crimping mechanism 602. The circular edges 610 of the disc-shaped rollers partially define a passage that extends axially through the crimping mechanism 602 along longitudinal axis a-a.

Each disc-shaped roller 606 is held in place in the radially arranged configuration by a connector 608 that is attached to crimping mechanism 602 via one or more fasteners 619, such that the location of each of the plurality of connectors is fixed with respect to the first end surface of the crimping mechanism 602. In the depicted example, fasteners 619 are positioned adjacent an outer portion of the crimping mechanism 602, radially outwardly of the disc-shaped rollers 606. Two fasteners 619 are used to position each connector 608 in the example shown, but the number of fasteners 619 can vary. As shown in FIGS. 25B and 25C, a connector 608 has a first arm 616 and a second arm 618. First and second arms 616, 618 extend over a disc-shaped roller 608 from a radially-outward portion of circular edge 610 to a central portion of the disc-shaped roller 608. A bolt 620 extends through the first and second arms 616, 618 and through a central lumen of the disc-shaped roller 608, the central lumen passing from a center point of front surface 612 to a center point of the back surface 614 of the disc-shaped roller 606 along central axis c-c. The bolt 620 is positioned loosely within the lumen, with substantial clearance/space to allow the disc-shaped roller 608 to rotate about central axis c-c.

During use, an elongated sheath is advanced from the first side 604 of the crimping mechanism 602, through the axial passage between the rollers, and out the second side 605 of the crimping mechanism 602. The pressure from the circular edge 610 of the disc shaped rollers 606 reduces the diameter of the sheath to a crimped diameter as it rolls along the outer surface of the elongated sheath.

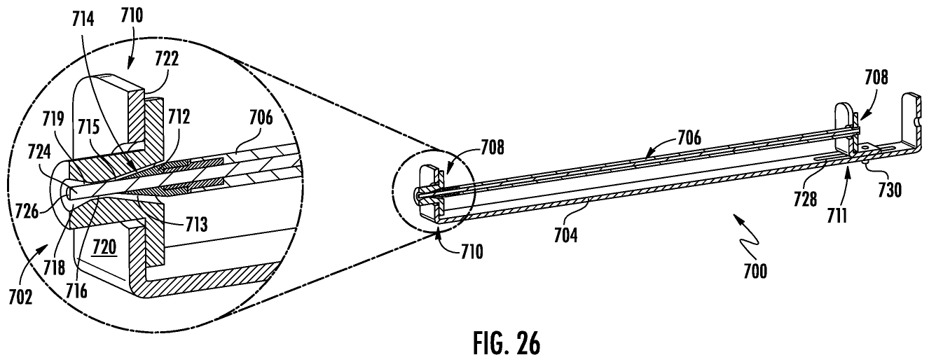

FIG. 26 shows an example of a crimping device 700 designed to facilitate crimping of elongated structures, such as sheaths. The crimping device includes an elongated base 704, and elongated mandrel 706 positioned above the elongated base 704, and a holding mechanism 708 attached to the elongated base 704. The holding mechanism 708 supports the mandrel 706 in an elevated position above base 704. The holding mechanism includes a first end piece 710 that includes a crimping mechanism 702. The mandrel 706 includes a conical end portion 712 that nests within a first tapered portion 713 of a narrowing lumen 714 of the first end piece 710. The conical end portion 712 of mandrel 706 is positioned loosely within the narrowing lumen 714, with enough space or clearance between the conical end portion 712 and the lumen 714 to allow for passage of an elongated sheath over the conical end portion 712 of mandrel 706 and through the narrowing lumen 714. During use, the conical end portion 712 helps to avoid circumferential buckling of the sheath during crimping. In some examples, the mandrel 706 can also include a cylindrical end portion 724 that extends outwardly from the conical end portion 712 and defines an end 726 of the mandrel 706.

The first tapered portion 713 of the narrowing lumen 714 opens toward a second end piece 711 of the holding mechanism 708, such that the widest side of the taper is located on an inner surface 722 of the first end piece 710. In the example shown, the first tapered portion 713 narrows to a narrow end 715 that connects with a narrow cylindrical portion 716 of the narrowing lumen 714. In this example, the narrow cylindrical portion 716 defines the narrowest diameter of the narrowing lumen 714. The cylindrical end portion 724 of the mandrel 706 may nest loosely within the narrow cylindrical portion 716 of the narrowing lumen 714, with enough space or clearance between the cylindrical end portion 724 and the narrow cylindrical portion 716 of the lumen to allow for passage of the elongated sheath. The elongated nature of the narrow cylindrical portion 716 may facilitate smoothing of the crimped sheath after it has passed over the conical end portion 712 of the mandrel. However, the length of the cylindrical portion 716 of the narrowing lumen 714 is not meant to limit the disclosure, and in some examples, the crimping mechanism 702 may only include first tapered portion 713 of the narrowing lumen 714, and still be effective to crimp an elongated sheath.

At the opposite end of the first end piece 710 shown in FIG. 26, a second tapered portion 718 of the narrowing lumen 714 opens up from narrow cylindrical portion 716 such that the widest side of the taper located on the outer surface 720 of the first end piece 710. The narrow end 719 of the second tapered portion 718 connects with the narrow cylindrical portion 716 of the narrowing lumen 714 in the interior of the crimping mechanism 702. The second tapered portion 718 of the narrowing lumen 714 may not be present in some examples.

The holding mechanism 708 further includes a second end piece 711 positioned opposite the elongated base 704 from the first end piece 710. The second end piece 711 is movable with respect to elongated base 704, such that the distance between the first end piece 710 and the second end piece 711 is adjustable and therefore able to support mandrels of varying sizes. In some examples, elongated base 704 may include one or more elongated sliding tracks 728. The second end piece 711 can be slidably engaged to the sliding track 728 via at least one reversible fastener 730, such as, but not limited to, a bolt that extends into or through the second end piece 711 and the elongated sliding track 728. To move the second end piece 711, the user would loosen or remove the reversible fastener 730, slide the second end piece 711 to the desired location, and replace or tighten the reversible fastener 730.

In use, a sheath in an uncrimped diameter can be placed over the elongated mandrel 706 of the crimping device 700 shown in FIG. 26, such that the inner surface of the entire length of the uncrimped sheath is supported by the mandrel. The uncrimped sheath is then advanced over the conical end portion 712 and through the narrowing lumen 714 of the crimping mechanism 702. The uncrimped sheath is crimped to a smaller, crimped diameter via pressure from the interior surface of the narrowing lumen 714. In some examples, the sheath is advanced through both a first tapered portion 713 and a cylindrical portion 716 of the narrowing lumen 714 before exiting the crimping mechanism 702. In some examples, the sheath is advanced through a first tapered portion 713, a cylindrical portion 716, and a second tapering portion 718 of the narrowing lumen 714 before exiting the crimping mechanism 702.

In some examples, the crimping mechanism 602 shown in FIG. 25A may be positioned within a larger crimping device such as crimping device 700 shown in FIG. 26. For example, the crimping mechanism 602 can be positioned within the first end piece 710 of crimping device 700 instead of, or in combination with, crimping mechanism 702. For example, the rolling crimping mechanism 602 could entirely replace the narrowing lumen 714 of crimping mechanism 702, or the rolling crimping mechanism 602 could be nested within the narrow cylindrical portion 716 of the narrowing lumen 714 of the crimping mechanism 702, such that the first tapered portion 713 feeds the expandable sheath through the plurality of radially arranged disc-shaped rollers 606.

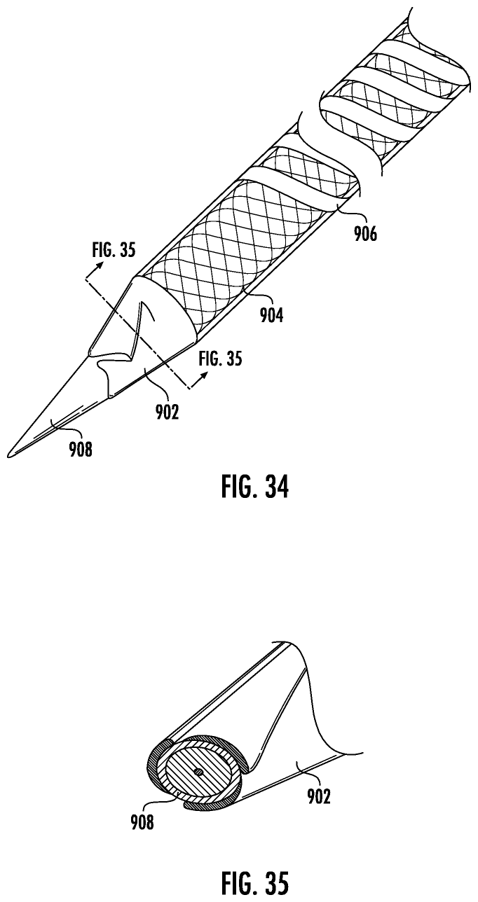

FIGS. 34-35 show an example sheath 100 including a distal end portion 902, which can be an extension of an outer cover extending longitudinally along the sheath 100 in the proximal direction. FIG. 34 shows a distal end portion 902 folded around an introducer 908 (in the crimped and collapsed configuration). FIG. 35 shows a cross section of the distal end portion 902 folded around the introducer 908 (in the crimped and collapsed configuration). The distal end portion 902 can be formed of, for example, one or more layers of a similar or the same material used to form the outer layer of the sheath 100. In some examples, the distal end portion 902 includes an extension of the outer layer of the sheath 100, with or without one more additional layers added by separate processing techniques. The distal end portion 902 can include anywhere from 1 to 8 layers of material (including 1, 2, 3, 4, 5, 6, 7, and 8 layers of material). In some examples, the distal end portion comprises multiple layers of a Dyneema® material. The distal end portion 902 can extend distally beyond a longitudinal portion of the sheath that includes braided layer 904 and elastic layer 906. In fact, in some examples, the braided layer 904 may extend distally beyond the elastic layer 906, and the distal end portion 902 may extend distally beyond both the braided layer 904 and elastic layer 906, as shown in FIGS. 34-35.

The distal end portion 902 may have a smaller collapsed diameter than the more proximal portions of the sheath, giving it a tapered appearance. This smooths the transition between the introducer/dilator 908 and the sheath 100, ensuring that the sheath 100 does not get lodged against the tissue during insertion into the patient. The smaller collapsed diameter can be a result of multiple folds (for example, 1, 2, 3, 4, 5, 6, 7, or 8 folds) positioned circumferentially (evenly or unevenly spaced) around the distal end portion. For example, a circumferential segment of the distal end portion 902 can be brought together and then laid against the adjacent outer surface of the distal end portion 902 to create an overlapping fold. In the collapsed configuration, the overlapping portions of the fold extend longitudinally along the distal end portion 902. Exemplary folding methods and configurations are described in U.S. application Ser. No. 14/880,109 (issued as U.S. Pat. No. 10,792,471) and U.S. application Ser. No. 14/880,111 (issued as U.S. Pat. No. 10,327,896), each of which are hereby incorporated by reference in their entireties. Scoring can be used as an alternative, or in addition to folding of the distal end portion 902. Both scoring and folding of the distal end portion 902 allow for the expansion of the distal end portion 902 upon the passage of the delivery system, and ease the retraction of the delivery system back into the sheath 100 once the procedure is complete. In some examples, the distal end portion 902 of the sheath 100 (and/or of the vessel dilator) can decrease from the initial diameter of the sheath (e.g., 8 mm) to 3.3 mm (10F), and may decrease to the diameter of a guide wire, allowing the sheath 100 and/or the vessel dilator 300 to run on a guide wire.

In some examples, a distal end portion 902 can be added, the sheath 100 and tip can be crimped, and the crimping of the distal end portion 902 and sheath 100 can be maintained, by the following method. As mentioned above, the distal end portion 902 can be an extension of the outer layer of the sheath 100. It can also be a separate, multilayer tubing that is heat bonded to the remainder of the sheath 100 prior to the tip crimping processing steps. In some examples, the separate, multilayer tubing is heat bonded to a distal extension of the outer layer of the sheath 100 to form the distal end portion 902. For crimping of the sheath 100 after tip attachment, the sheath 100 is heated on small mandrel. The distal end portion 902 can be folded around the mandrel to create the folded configuration shown in FIG. 34. The folds be added to the distal end portion 902 prior to the tip crimping process, or at an intermediate point during the tip crimping process. In some examples, the small mandrel can be from about 2 millimeters to about 4 millimeters in diameter (including about 2.2 millimeters, about 2.4 millimeters, about 2.6 millimeters, about 2.8 millimeters, about 3.0 millimeters, about 3.2 millimeters, about 3.4 millimeters, about 3.6 millimeters, about 3.8 millimeters and about 4.0 millimeters). The heating temperature will be lower than the melting point of the material used. This can cause the material to shrink on its own to a certain extent. For example, in an example sheath where Dyneema® materials are utilized as part of the sheath 100 outer layer and/or distal end portion 902 materials, a sheath crimping process begins by heating the sheath 100 on a 3 millimeter mandrel to about 125 degrees Celsius (lower than Dyneema® melting point of about 140 degrees Celsius). This causes the sheath 100 to crimp itself to about a 6 millimeter outer diameter. At this point, the sheath 100 and distal end region 902 are allowed to cool. A heat shrink tube can then be applied. In some examples, the heat shrink tube can have a melting point that is about the same as the melting point of the distal end portion 902 material. The sheath 100 with the heat shrink tube extending over the sheath 100 and the distal end portion 902 is heated again (for example, to about 125 degrees Celsius for sheaths including Dyneema® outer layers and distal end portions), causing the sheath 100 to crimp to an even smaller diameter. At the distal end portion 902, a higher temperature can be applied (for example, from about 145 degrees Celsius to about 155 degrees Celsius for Dyneema® material) causing the layers of material to melt together in the folded configuration shown in FIG. 34 (the folds can be added at any point during this process). The bonds at the distal end portion 902 induced by the high temperature melting step will still be weak enough to be broken by a passing delivery system. As a final step, the heat shrink tube is removed, and the shape of the sheath 100 remains at the crimped diameter.

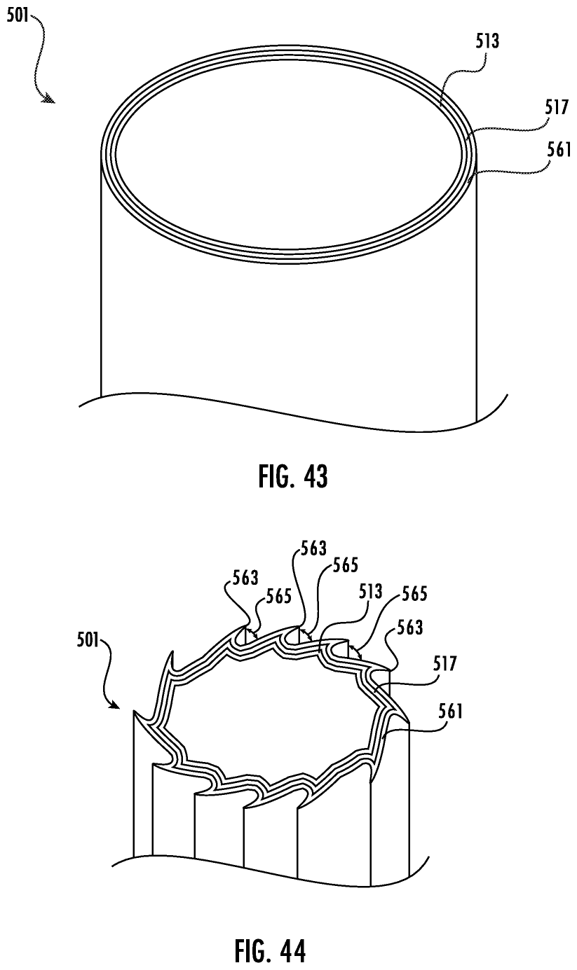

FIG. 43 shows a transverse cross section taken near the distal end of another sheath example, at a point longitudinally distal to the braided layer. The sheath 501 includes an inner polymeric layer 513, an outer polymeric layer 517, and an outer covering 561. A method of compressing the distal portion of an expandable sheath 501 can include: covering at pre-crimped state the distal portion of the expandable sheath 501 with an external covering layer 561 having a melting temperature TM1 which is lower than the melting temperature TM2 of the inner and outer polymeric layers; heating at least one region, which does not span the entire area of overlap between the cover layer 561 and the expandable sheath 501, to a first temperature which is equal or higher than TM2, thereby melting both the covering layer 561 and the outer polymeric layer 517 of the expandable sheath 501, so as to create at attachment region 569 there between; inserting a mandrel into the lumen of the expandable sheath 501 and crimping at least a portion thereof, such as the distal portion, of the expandable sheath 501; heating the external covering layer 561 over the distal portion of the expandable sheath 501 to a second temperature which is at least equal to or higher than the melting temperature TM1 of the external covering layer 561, and lower than the melting temperature TM2 of the inner and outer polymeric layers, for a predefined first time window.

This method advantageously avoids risks that a tear initiated at a score or split line (such as perforation 813 shown in FIG. 29) should divert from the intended axial direction of tear propagation due to defects (weakened points or unintended apertures) in the heat-shrink tubing. This method further enables choosing an external covering layer made of materials that may be heated to form moderately attached folds at temperatures lower than those required for the internal or external layers of the expandable sheath.

The crimping of the inner and outer polymeric layers (e.g., inner and outer layers 513, 517) and the external covering layer (e.g., layer 561) can be, for example, from a pre-compressed diameter of about 8.3 mm to a compressed diameter of about 3 mm. FIG. 44 shows a transverse cross section of the example of FIG. 43 during crimping. Folds 563 are created along the external layer 561 during crimping. The heating to the second temperature is sufficient to melt the external covering layer 561 so as to attach the fold 563 to each other, while avoiding similar melting and attachment of the inner and outer polymeric layers.

The method of compressing the distal portion of the expandable sheath can further include a step of covering the expandable sheath 501 and the external covering layer 561 with a heat-shrink tube (HST) prior to, during or following the heating to the second temperature, wherein the second temperature further acts to shrink the HST in order to retain the external covering layer 561 and the expandable sheath 501 in a compressed state. The HST can be removed from the expandable sheath 501 and the external covering layer 561 after the folds 563 of the covering layer 563 are sufficiently attached to each other in the desired compressed state, and cooled down for a sufficient period of time.

According to some examples, the HST is further utilized as a heat shrink tape, to apply the external radial pressure by wrapping and heating it over the external covering layer 561 and the expandable sheath 501. According to some examples, a non heat-shrink tape can be used instead of a heat shrink tube.

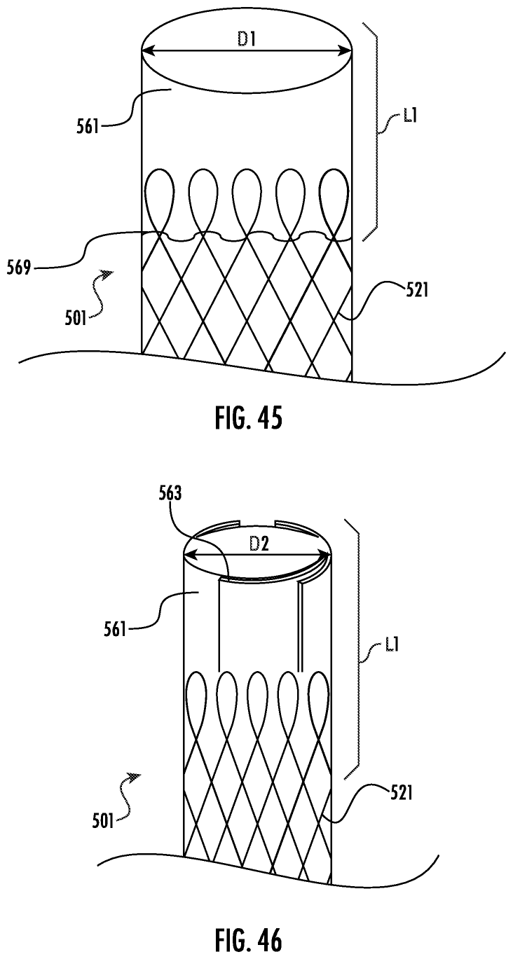

FIG. 45 shows a distal portion of an expandable sheath 501 having an expandable braid 120, wherein its distal portion is covered by an external covering layer 561, which is shown to extend along a length L1 up to the distal edge 513 of the expandable sheath 501. D1 denotes the distal diameter of the expandable sheath 501 in the pre-compressed state. FIG. 6B shows the distal portion of the expandable sheath 501 in a compressed state, wherein its distal diameter D2 is smaller than D1. It should be noted that compressing the external covering layer 561, from an un-compressed state to a compressed state of the expandable sheath 501, results in formation of folds 563 (FIGS. 44 and 46) along the external covering layer 561 as well as layers 517 and 513, when reaching the compressed state, due to the diameter reduction thereof. It is desirable to promote moderate attachment between the folds 563. The term “moderate attachment”, as used herein, refers to an attachment force sufficient in magnitude to form a structural cover maintaining the expandable sheath 501in a compressed state prior to advancement of a DS component through its lumen, yet low enough so that advancement of the DS component there-through is sufficient to break or disconnect the attachments 565 between the folds 563 (FIG. 44), thereby enabling expansion of the expandable sheath 501.

The external covering layer 561 is chosen such that its melting temperature TM1 is lower than the melting temperature TM2 of the polymeric layers of the expandable sheath 100, in order to promote folds 563 formation with moderate attachment in the external covering layer 561, while avoiding melting and attaching similar folds in the polymeric layers 513 and 517 of the expandable sheath 501.

According to some examples, the external covering layer 561 is low density polyethylene. Other suitable materials as known in the arts, such as polypropylene, thermoplastic polyurethane, and the like, may be utilized to form the external covering layer 561.

FIGS. 45 and 46 show perspective views of an example sheath that is similar to or the same as FIGS. 43 and 44. The external covering layer 561 and expandable sheath 501 were heated to a first temperature TM2 along a circumferential interface there between at the proximal end of the external covering layer 561, to form a circumferential proximal attachment region 569.

According to some examples, the external covering layer 561 is attached different attachment regions, such as along a longitudinally oriented attachment line, to the external surface of the expandable sheath 501 (e.g., the outer polymeric layer). According to some examples, the external covering layer 561 is attached to the external surface of the expandable sheath 501 by a plurality of circumferentially spaced attachment regions 569, 571, wherein the circumferential distance between adjacent attachment regions is chosen to allow formation of folds 573 there between. Attachment regions 569, 571 ensure that the external covering layer 561 always remains attached to the expandable sheath 501, either during the compressed or expanded states thereof.

According to some examples, the covering with an external covering layer 561 is performed after crimping the expandable sheath 501, such that the external layer 561 covers pre-formed folds of inner 513 and/or outer 517 layers of the sheath 501.

According to some examples, the bond between the folds 563 is based on adhesive with moderate adhesion strength.

Examples of the sheaths described herein may comprise a variety of lubricious outer coatings, including hydrophilic or hydrophobic coatings, and/or surface blooming additives or coatings.

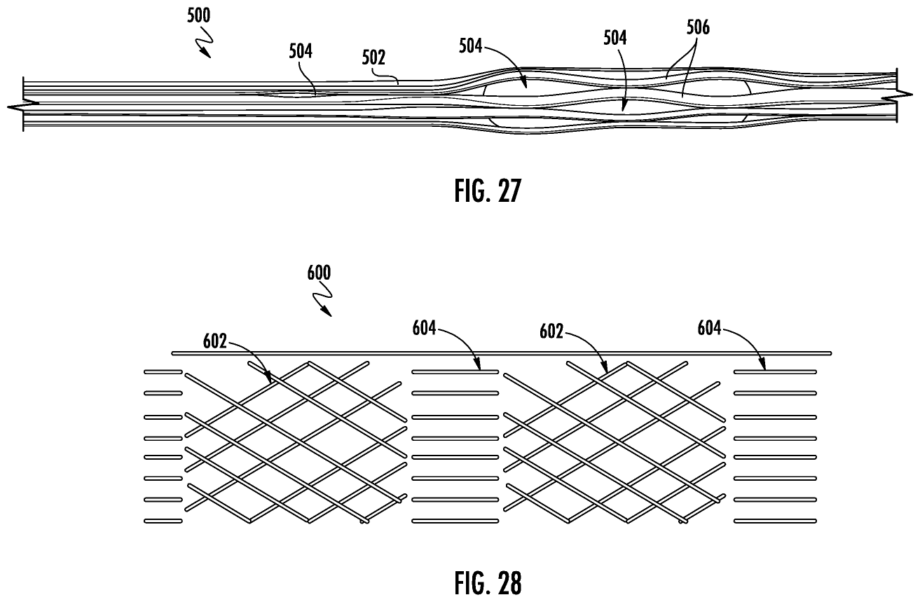

FIG. 27 illustrates another example of a sheath 500 comprising a tubular inner layer 502. The inner layer 502 may be formed from an elastic thermoplastic material such as nylon, and can comprise a plurality of cuts or scorelines 504 along its length such that the tubular layer 502 is divided into a plurality of long, thin ribs or portions 506. When the delivery apparatus 10 is advanced through the tubular layer 502, the scorelines 504 can resiliently expand or open, causing the ribs 506 to splay apart, and allowing the diameter of the layer 502 to increase to accommodate the delivery apparatus.

In other examples, the scorelines 504 can be configured as openings or cutouts having various geometrical shapes, such as rhombuses, hexagons, etc., or combinations thereof. In the case of hexagonal openings, the openings can be irregular hexagons with relatively long axial dimensions to reduce foreshortening of the sheath when expanded.

The sheath 500 can further comprise an outer layer (not shown), which can comprise a relatively low durometer, elastic thermoplastic material (e.g., Pebax, polyurethane, etc.), and which can be bonded (e.g., by adhesive or welding, such as by heat or ultrasonic welding, etc.) to the inner nylon layer. Attaching the outer layer to the inner layer 502 can reduce axial movement of the outer layer relative to the inner layer during radial expansion and collapse of the sheath. The outer layer may also form the distal tip of the sheath.

FIG. 28 illustrates another example of a braided layer 600 that can be used in combination with any of the example sheaths described herein. The braided layer 600 can comprise a plurality of braided portions 602, in which filaments of the braided layer are braided together, and unbraided portions 604, in which the filaments are not braided, and extend axially without being intertwined. In certain examples, the braided portions 602 and unbraided portions 604 can alternate along the length of the braided layer 600, or may be incorporated in any other suitable pattern. The proportion of the length of the braided layer 600 given to braided portions 602 and unbraided portions 604 can allow the selection and control of the expansion and foreshortening properties of the braided layer.

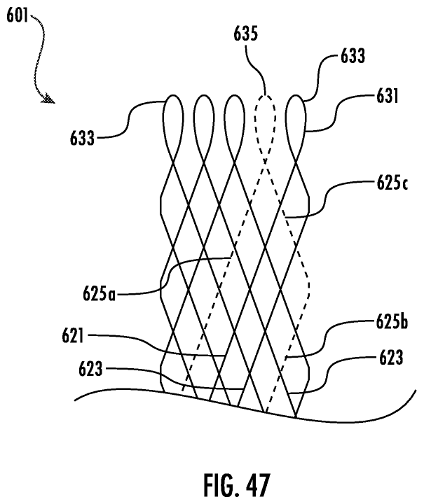

FIG. 47 depicts an example of a braided layer 601 having at least one radiopaque strut or filament. The expandable sheath 601 and its expandable braided layer 621 is shown without the polymeric layers, as would be visualized in the x-ray fluoroscopy, for purposes of illustration. As shown in FIG. 47, the expandable braided layer 621 comprises a plurality of crossing struts 623, that can further form distal crowns 633, for example in the form of distal loops or eyelets at the distal portion of the expandable sheath 601.

The expandable sheath 601 is configured for advancement in a pre-compressed state up to a target area, for example along the abdominal aorta or the aortic bifurcation, at which point the clinician should cease further advancement thereof and introduce the DS through its lumen, to facilitate expansion thereof. For that end, the clinician should receive a real-time indication of the expandable sheath's position during advancement thereof. According to an aspect of the present disclosure, there is provided at least one radio-opaque marker at or along at least one region of the expandable braided layer 621, configured to enable visualization of the expandable sheath's position under radio fluoroscopy.

According to one example, at least one of the distal crowns 633 comprises a radio-opaque marker. According to some examples, the distal crowns 633 comprise at least one gold-plated crown 635 (FIG. 47), configured to serve as a radio-opaque marker. It will be clear that gold-plating is merely an example, and that the crowns 635 can comprise other radio-opaque material known in the art, such as tantalum, platinum, iridium and the like.

Since the expandable sheath 601 comprises an expandable braided layer 621 having a plurality of crossing struts 623 disposed along its length, this structure can be advantageously utilized for more convenient incorporation of radio-opaque elements.

According to some examples, the struts 623 further comprise at least one radio-opaque strut 625, having a radio-opaque core. For example, a drawn filled tubing (DFT) wire comprising a gold core (as may be provided by, for example, Fort Wayne Metals Research Products Corp.) may serve as a radio-opaque strut 625. FIG. 47 shows an exemplary expandable braided layer 621 comprising a plurality of less-opaque struts or filaments 623 and radio-opaque struts or filaments 625a, 625b and 625c. In some instances, the struts 625a and 625c can be made of a single wire, wherein the wire extends along the path of strut 625a, loops at the distal crown 635 and extends along the path of strut 625c therefrom. Thus, a single wire, such as a DFT wire, can be utilized to form radio-opaque struts 625a and 625c and radio-opaque distal crown 635.

Since radio-opaque wires, such as a DFT wire, can be costly, the expandable braided layer 621 can comprise a plurality of non radio-opaque or less radio-opaque struts 623, for example made of a shape-memory alloy such as Nitinol and polymer wire such as PET respectively, intertwined with at least one radio-opaque strut 625 (FIG. 47).

According to some examples, radio-opaque wires are embedded within the polymer braid, such as the outer polymeric layer 617 or the inner polymeric layer 615, which are made of less-opaque materials.

Advantageously, the expandable braid embedded within the expandable sheath is utilized according to the present disclosure, for incorporating radio-opaque markers along specific portions thereof to improve visualization of the sheath's position in real-time under radio fluoroscopy.

According to yet another aspect of the present disclosure, radiopaque tubes can be threaded on the distal crowns or loops 633, or radiopaque rivets can be swaged on the distal crowns or loops 633 to improve their visibility under fluoroscopy.

FIG. 36 shows a longitudinal cross section of another example of expandable sheath 11 (positioned on mandrel 91 during the fabrication process, under compression by heat shrink tube 51). The sheath 11 comprises a braided layer 21, but lacks the elastic layer described in previous examples. The heat applied during the shrinking procedure may promote at least partial melting of the inner 31 and outer 41 polymeric layers. Since the filaments of the braid define open cells therebetween, uneven outer surfaces may be formed when the inner 31 and outer 41 polymeric layers melt into the cell openings and over the filaments of the braided layer 21.

In order to mitigate uneven surface formations, cushioning polymeric layers 61a, 61b are added between the inner 31 and outer 41 layers of the sheath 11, configured to evenly spread the forces acting in the radial direction during sheath compression. A first cushioning layer 61a is placed between the inner polymeric layer 31 and the braided layer 21, and a second cushioning layer 61b is placed between the outer polymeric layer 41 and the braided layer 21.

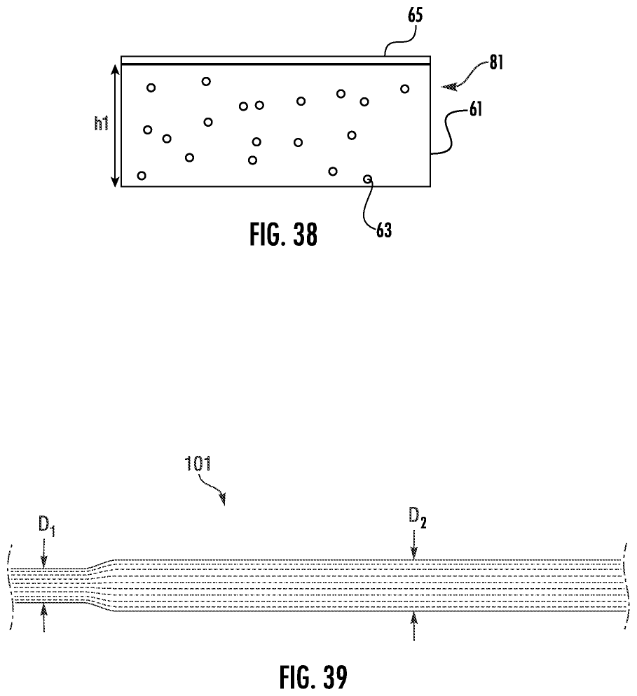

The cushioning layers 61a, 61b can comprise a porous material having plurality of micropores of nanopores 63 (FIGS. 37-38) in a porous interior region. One such material includes, but is not limited to, expanded polytetrafluoroethylene (ePTFE). A porous cushioning layer can advantageously be formed with a minimal thickness h1 required to sufficiently spread the compression forces to prevent uneven surface formation along the inner 31 and outer 41 polymeric layers. Thickness h1 is measured in the radial direction (from an inner surface to an outer surface) of the cushioning layer, and can be from about 80 microns to about 1000 microns (including, for example, about 80 microns, about 90 microns, about 100 microns, about 110 microns, about 120 microns, about 130 microns, about 140 microns, about 150 microns, about 160 microns, about 170 microns, about 180 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, and about 1000 microns). In some examples, the range of thickness h1 is from about 110 to 150 microns.

However, when cushioning layers comprise a plurality of micropores of nanopores 63 (FIGS. 37-38), the inner 31 and outer 41 polymeric layers may melt into the pores of the cushioning layers 61a, 61b upon heating during the fabrication process. In order to prevent the inner 31 and outer 41 polymeric layers from melting into the pores 63 of the cushioning layer 61, a first sealing layer 71a can be placed between the inner polymeric layer 31 and the first cushioning layer 61a, and a second sealing layer 71b can be placed between the outer polymeric layer 41 and the second cushioning layer 61b. (as shown in FIG. 36). The sealing layers 71a, 71b can have a higher melting point than the polymeric layers 31 and 41, and can be formed of a non-porous material (such as, but not limited to, polytetrafluoroethylene) in order to prevent fluid flow therethrough. The thickness h2 of each sealing layer 71 (FIG. 37), measured in a radial direction from the inner to the outer surface of the sealing layer, can be much thinner than that of the cushioning layer 61, for example, from about 15 to about 35 microns (including about 15 microns, about 20 microns, about 25 microns, about 30 microns, and about 35 microns).

While advantageous for the reasons described above, the addition of the cushioning and sealing can increase the complexity and time required to assemble the sheath 11. Advantageously, providing a single sealed cushioning member, configured to provide both cushioning and sealing functionalities (instead of providing two separate cushioning and sealing layers, each configured to provide one functionality) reduces sheath assembly time and significantly simplifies the process. According to an aspect of the present disclosure, there is provided a single sealed cushioning member, configured for placement between the inner and outer polymeric layers of the sheath and the central braided layer. The single sealed cushioning member includes cushioning layer and a sealed surface configured to prevent leakage/melting into the pores in the radial direction.

FIG. 37 shows an example of a single sealed cushioning member 81′, comprising a cushioning layer 61 having a width thickness h1 as elaborated herein above, fixedly attached to a corresponding sealing layer 71 having a thinner thickness h2 to form the sealed surface. The sealing layer 71 and the cushioning layer 61 are pre-assembled or pre-attached to each other to form together a single member 81′, for example by gluing, welding and the like.

FIG. 38 shows one example of a single sealed cushioning member 81, comprising a cushioning layer 61 having a width thickness h1, wherein the cushioning layer 61 is provided with at least one sealed surface 65, configured to face an inner 31 or an outer 41 polymeric layer when assembled in the sheath 11. According to some examples, the sealed surface 65 can be formed by a surface treatment configured to fluidly seal a surface of the cushioning layer 61. As such, the sealed surface 65 can be the same material as the cushioning layer 61.

According to another aspect of the present disclosure, and as mentioned above with respect to FIG. 36, a minimum of three layers may be sufficient to retain the sheath's expandability provided with the preferable resistance to axial elongation. This is accomplished by eliminating the need to incorporate an additional elastic layer in the sheath, thereby advantageously reducing production costs and simplifying manufacturing procedures. The sheath does not necessarily return to an initial diameter, but may rather remain in an expanded diameter upon passage of the valve, in the absence of the elastic layer.

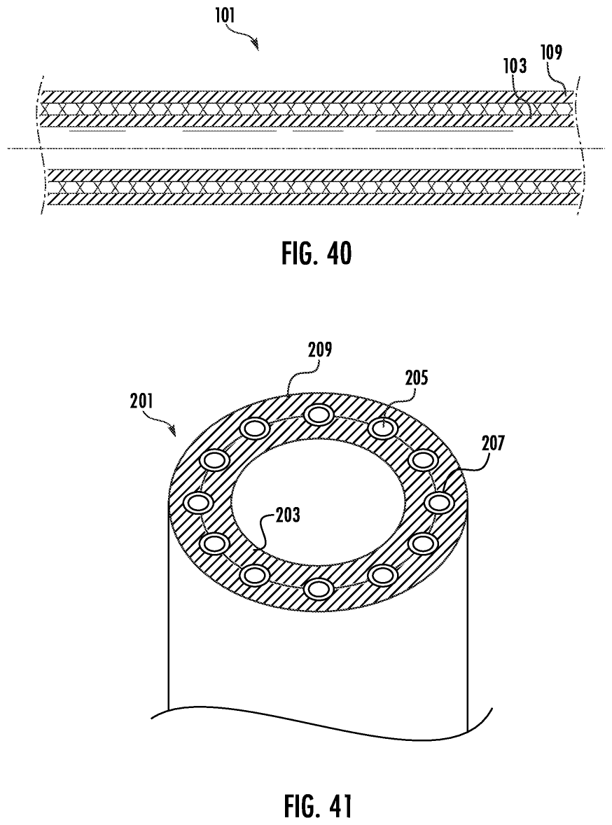

FIGS. 39-40 show an expandable sheath 101 similar to the expandable sheath 100 shown in FIG. 3, but without an elastic layer 106. Sheath 301 includes structure similar to sheath 100 described above, like element numbers are used to designate like structure. The inner and outer layers 103 and 109 may be structured and configured to resist axial elongation of the sheath 101 during expansion. However, in the proposed configuration, the absence of an elastic layer results in the sheath 101 remaining in an expanded diameter along the sheath's portion proximal to the valve, without necessarily collapsing back to the initial diameter D₁ after the valve passes in in the longitudinal direction. FIG. 39 is a schematic representation of the sheath 101 remaining in an expanded diameter D₂ along the portion proximal to the valve's passage.

Thus, there is provided an expandable sheath for deploying a medical device, comprising a first polymeric layer, a braided layer radially outward of the first polymeric layer, and a second polymeric layer radially outward of the braided layer. The braided layer includes a plurality of filaments braided together. The second polymeric layer is bonded to the first polymeric layer such that the braided layer is encapsulated between the first and second polymeric layers. When a medical device is passed through the sheath, the diameter of the sheath expands from a first diameter to a second diameter around the medical device, while the first and second polymeric layers resist axial elongation of the sheath such that the length of the sheath remains substantially constant. However, according to some examples, the first and second polymeric layers are not necessarily configured to resist axial elongation.

According to another aspect of the present disclosure, the expandable sheath does include an elastic layer. But, unlike elastic layer 106 shown in FIG. 3, the elastic layer is not configured to apply a substantial radial force. It can still serve to provide column strength to the sheath. By limiting tangential (diametrical) expansion of the braid, the elastic layer enhances the strength of the braid and the sheath in the axial direction (column strength). As such, use of elastic materials with higher tensile strengths (resistance to stretch) will result in a sheath with greater column strength. Likewise, elastic materials that are under greater tension in the free state will also result in a sheath with greater column strength during pushing, as they will be more resistant to stretch. The pitch of any helically wound elastic layers is another variable that contributes to the column strength of the sheath. The additional column strength ensures that the sheath does not spontaneously expand due to frictional forces applied thereto during forward movement in a distal direction, and does not buckle when the delivery system is pulled out of the sheath.

In another optional example, the elastic layer can be applied by dip coating in an elastic material (such as, but not limited to) silicone or TPU. The dip coating can be applied to the polymeric outer layer, or to the braided layer.

Thus, there is provided an expandable sheath for deploying a medical device, comprising a first polymeric layer, a braided layer radially outward of the first polymeric layer, an elastic layer radially outward of the braided layer, and a second polymeric layer radially outward of the braided layer. The braided layers comprise a plurality of filaments braided together. The elastic layer is configured to provide the expandable sheath with sufficient column strength to resist buckling of spontaneous expansion due to friction forces applied thereto by a surrounding anatomical structure during the sheath's movement in an axial direction. The second polymeric layer is bonded to the first polymeric layer such that the braided layer is encapsulated between the first and second polymeric layers. When a medical device is passed through the sheath, the diameter of the sheath expands from a first diameter to a second diameter around the medical device, optionally while the first and second polymeric layers resist axial elongation of the sheath such that the length of the sheath remains substantially constant.

According to an aspect of the present disclosure, there is provided a three-layered expandable sheath, comprising an inner polymeric layer, an outer polymeric layer bonded to the inner polymeric layer and a braided layer encapsulated between the inner and outer polymeric layers, wherein the braided layer comprises an elastic coating.

FIG. 41 shows a transverse cross section of expandable sheath 201. Sheath 201 includes structure similar to sheath 100 described above, like element numbers are used to designate like structure. The expandable sheath 201 includes inner and outer polymeric layers 203 and 209 and a braided layer 205. Instead of the elastic layer described with reference to FIG. 3, above, the braided layer 205 is provided with an elastic coating 207. The elastic coating 207 can be applied directly to the filaments of the braided layer 205, as shown in FIG. 41. The elastic coating can be made of synthetic elastomers, exhibiting properties similar to those described in conjunction with the elastic layer 106.

In some examples, the second, outer polymeric layer 209 is bonded to the first, inner polymeric layer 203 such that the braided layer 205 and the elastic coating 207 are encapsulated between the first and second polymeric layers. Moreover, the elastic coating applied directly to the braided filaments is configured to serve the same function as that of the elastic layer 106 (that is, to apply radial force on the braided layer and the first polymeric layer).

While the example of FIG. 41 shows the elastic coating 207 covering the entire circumference of every filament of the braided layer 205, it will be understood that only a portion of the filaments, for example, a portion constituting essentially an outer surface of the braided layer, may be coated by the elastic coating 207.

Alternatively, or additionally, an elastic coating can be applied to other layers of the sheath 201.

In some examples, a braided layer such as the one shown in FIG. 40 can have self-contractible frame made of a shape-memory material, such as, but not limited to, Nitinol. The self-contracting frame can be pre-set to have a free-state diameter equal to the sheath's initial compressed diameter D1, for example, prior to being placed on a mandrel around the first polymeric layer. The self-contracting frame may expand to a larger diameter D2 while an inner device, such as a prosthetic valve, passes through the sheath's lumen, and self-contract back to the initial diameter D1 upon passage of the valve. In some examples, the filaments of the braid are the self-contracting frame, and are made of a shape-memory material.

According to another aspect, an expandable sheath can include a braided expandable layer, attached to at least one expandable sealing layer. In some examples, the braided layer and the sealing layer are the only two layers of the expandable sheath. The braided layer is passively or actively expandable relative to a first diameter, and the at least one expandable sealing layer is passively or actively expandable relative to a first diameter. An expandable sealing layer can be useful with any of the examples described above, and may be particularly advantageous for braids having self-contracting frames or filaments.

The braided layer can be attached or bonded to the expandable sealing layer along its entire length, advantageously decreasing the risk of the polymeric layer being peeled off the braided layer due to frictional forces that may be applied thereon either during entry or exit through the surgical incision. The at least one sealing layer can comprise a lubricious, low-friction material, to facilitate passage of the sheath within the blood vessels, and or to facilitate passage of the delivery apparatus carrying a valve through the sheath.

A sealing layer is defined as a layer which is not permeable to the blood flow. The sealing layer can comprise a polymeric layer, a membrane, a coating and/or a fabric, such as a polymeric fabric. According to some examples, the sealing layer comprises a lubricious, low-friction material. According to some examples, the sealing layer is radially outward to the braided layer, to facilitate passage of the sheath within the blood vessels. According to some examples, the sealing layer is radially inward to the braided layer, to facilitate passage of the medical device through the sheath.

According to some examples, the at least one sealing layer is passively expandable and/or contractible. In some examples, the sealing layer is thicker at certain longitudinal positions of the sheath than at others, which can hold a self-contracting braided layer open at a wider diameter than at other longitudinal positions where the sealing layer is thinner.

Attaching the braided layer to at least one expandable sealing layer, instead of encapsulating it between two polymeric layers bonded to each other, may simplify manufacturing process and reduce costs.

According to some examples, the braided layer can be attached to both an outer expandable sealing layer and an inner expandable sealing layer, so as to seal the braided layer from both sides, while facilitating passage of the sheath along the blood vessels, and facilitating passage of a medical device within the sheath. In such examples, the braided layer can be attached to a first sealing layer, while the other sealing layer may be also attached to the first sealing layer. For example, the braided layer and the inner sealing layer can be each attached to the outer sealing layer, or the braided layer and the outer sealing layer can be each attached to the inner sealing layer.

According to some examples, the braided layer is further coated by a sealing coating. This may be advantageous in configurations of a braided layer being attached only to a single expandable layer, wherein the coating ensures that the braided layer remains sealed from the blood flow or other surrounding tissues, even along regions which are not covered by the expandable layer. For example, if a braided layer is attached to a sealing layer on one side, the other side of the braided layer may receive a sealing coating. In some examples, the sealing coating can be used instead of, or in addition to, one or both of the sealing layers.

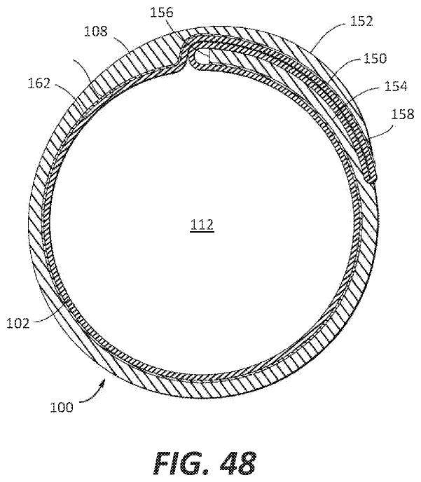

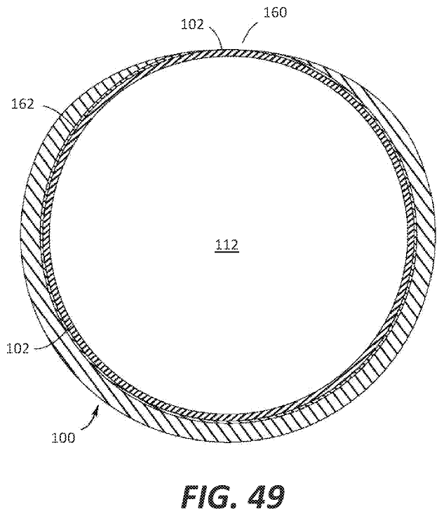

In another example, the sheath 100 can include a folded inner and outer layer construction. Example sheaths are described for example in U.S. application Ser. No. 12/249,867 filed Oct. 10, 2008 (issued as U.S. Pat. No. 8,690,936), U.S. application Ser. No. 13/312,739 file Dec. 6, 2011 (issued as U.S. Pat. No. 8,790,387), the disclosures of which are incorporated herein by reference in their entirety. FIGS. 48 and 49 illustrate a cross-section of an alternate sheath construction. The sheath 100 includes an inner layer 102 and an outer layer 108 as described above. In some examples, a thin layer of bonding or adhesive material 162 is positioned between the inner and outer layers 102, 108. As shown in FIG. 48, the inner layer 102 can be arranged to form a substantially cylindrical lumen 112 therethrough. Inner layer 102 can include one or more folded portions 150. In the example shown in FIG. 48, inner layer 102 is arranged to have one folded portion 150 that can be positioned on either side of the inner layer 102. Inner layer 102 can be continuous, in that there are no breaks, slits, or perforations in inner layer 102. Outer layer 108 can be arranged in an overlapping fashion such that an overlapping portion 120 overlaps at least a part of the folded portion 150 of the inner layer 102. As shown in FIG. 48, the overlapping portion 152 also overlaps an underlying portion 154 of the outer layer 108. The underlying portion 154 can be positioned to underlie both the overlapping portion 152 of the outer layer 108, as well as the folded portion 150 of the inner layer 102. Thus, the outer layer 108 can be discontinuous, in that it includes a slit or a cut in order to form the overlapping and underlying portions 152, 154. In other words, a first edge 156 of the outer layer 108 is spaced apart from a second edge 158 of the outer layer 108 so as not to form a continuous layer. The configuration of FIG. 48 allows for radial expansion of the sheath 100 as an outwardly directed radial force is applied from within (e.g., by passing a medical device such as a prosthetic heart valve through the lumen 112). As radial force is applied, the folded portion 150 can at least partially separate, straighten, and/or unfold, and/or the overlapping portion 152 and the underlying portion 154 of the outer layer 108 can slide circumferentially with respect to one another, thereby allowing the diameter of lumen 112 to enlarge as shown in FIG. 49.

In this manner, the sheath 100 is configured to expand from a resting configuration (FIG. 48) to an expanded configuration shown in FIG. 48. In the expanded configuration, as shown in FIG. 49, an annular gap 160 can form between the longitudinal edges of the overlapping portion 152 and the underlying portion 154 of the outer layer 108. As the sheath 100 expands at a particular location, the overlapping portion 152 of the outer layer 108 can move circumferentially with respect to the underlying portion 154 as the folded portion 150 of the inner layer 102 unfolds. This movement can be facilitated by the use of a low-friction material for inner layer 102, such as PTFE. Further, the folded portion 150 can at least partially separate and/or unfold to accommodate a medical device having a diameter larger than that of lumen 112 in the resting configuration. As shown in FIG. 48, in some examples, the folded portion of the inner layer 108 can completely unfold, so that the inner layer 102 forms a cylindrical tube at the location of the expanded configuration.

The sheath 100 can be configured such that it locally expands at a particular location corresponding to the location of the medical device along the length of the lumen 112, and then locally contracts once the medical device has passed that particular location. Thus, a bulge may be visible, traveling longitudinally along the length of the sheath as a medical device is introduced through the sheath, representing continuous local expansion and contraction as the device travels the length of the sheath 100. In some examples, each segment of the sheath 100 can locally contract after removal of any radial outward force such that it regains the original resting diameter of lumen 112. In some examples, each segment of the sheath 100 can locally contract after removal of any radial outward force such that it at least partially returns to the original resting diameter of lumen 112.

It is contemplated that an expandable introducer can be used with any of the sheath examples described herein. As described above, the various sheath examples can include a distal tip formed from multiple layers of material and/or folded layers treated with compression or heating. As a result, the distal tip of the sheath may be narrower and/or stiffer than the remaining portions of the sheath. Accordingly, an expandable introducer may be used to facilitate expansion of the distal tip of the sheath/pre-dilate the sheath tip, proving more room for delivery and withdraw of medical equipment through the distal opening of the sheath. Accordingly, we are able to reduce the push force needed for delivery and provide an opening large enough to retrieve medical equipment without causing trauma to the patient and/or damage to the sheath.

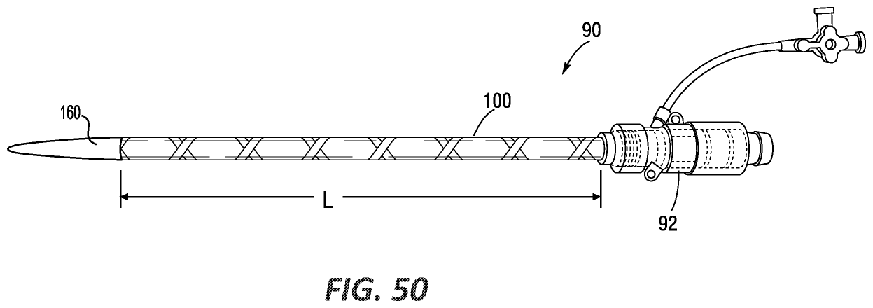

FIG. 50 illustrates an expandable introducer sheath 100 in combination with an expandable introducer 160, according to one example. While the expandable introducer 160 is illustrated in conjunction with the sheath 100 of FIG. 2, it is contemplated that the expandable introducer 160 can be used with any of the expandable sheath examples described above. Similar to vessel dilator 300 described above, the introducer 160 facilitates expansion of the distal tip portion of the sheath 100, and the corresponding area of the patient's blood vessel.

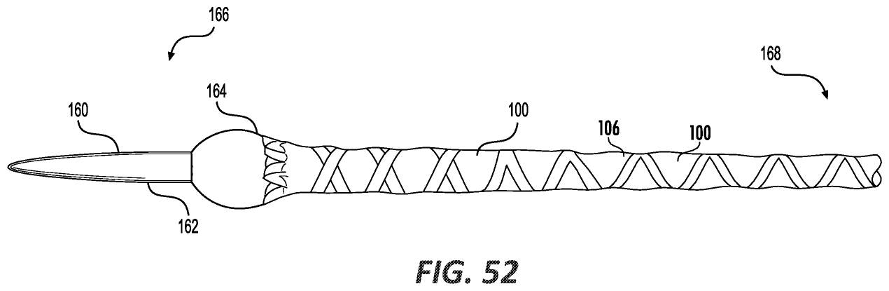

As illustrated in FIG. 50, the introducer 160 is received within a central lumen 112 of the sheath 100. The introducer 160 includes an elongated body member 162 and one or more inflatable members, e.g., balloon(s) 164, disposed along the elongated body member 162 between proximal and distal ends 166, 168. The balloon 164 is configured to expand radially in a direction away from the outer surface of the elongated body member 162. As will be described in more detail below, when the balloon 164 is inflated, the distal end portion 140 of the sheath 100 expands providing an enlarged distal opening (FIG. 52).

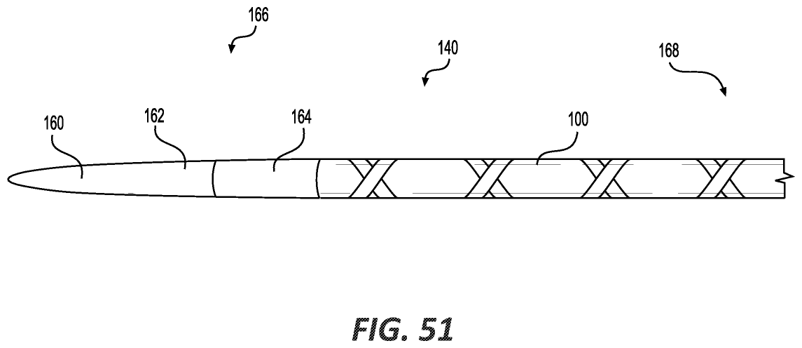

The balloon 164 is inflatable from an unexpanded configuration (FIGS. 50 and 51) to an expanded configuration (FIG. 52). In the unexpanded/deflated configuration, the outer diameter of the balloon 164 corresponds to the outer diameter of the elongated body member 162. In the unexpanded/deflated configuration, the outer diameter of the balloon 164 can also be less than the outer diameter of the elongated body member 162. In the inflated/expanded configuration, the outer diameter of the balloon 164 is greater than the outer diameter of the elongated body member 162. In the inflated/expanded configuration, the diameter of the balloon 164 is up to about 75% larger than the diameter of the diameter of the elongated body member 162. In some examples, in the inflated/expanded configuration, the diameter of the balloon 164 is about 10% to about 25% larger than the diameter of the elongated body member 162. In an example system, the maximum diameter of the balloon 146 in the inflated configuration is about 25% to about 50% larger than the maximum diameter of the elongated body member 162. In further examples, the maximum diameter of the balloon 146 in the inflated configuration is about 50% to about 75% larger than the maximum diameter of the elongated body member 162.

The introducer 160 is both axially/longitudinally and rotatably movable within the central lumen 112. As illustrated in FIGS. 50-51, the introducer 160 is movable through the distal opening provided in the introducer sheath 100. When the balloon 164 is deflated, at least a portion of the balloon 164 can pass through a distal opening of the introducer sheath 100 (FIG. 51). Once inflated/expanded, the balloon 164 dilates/expands the distal end portion 140 of the sheath 100 (or at least a portion thereof), increasing a diameter of the distal opening.

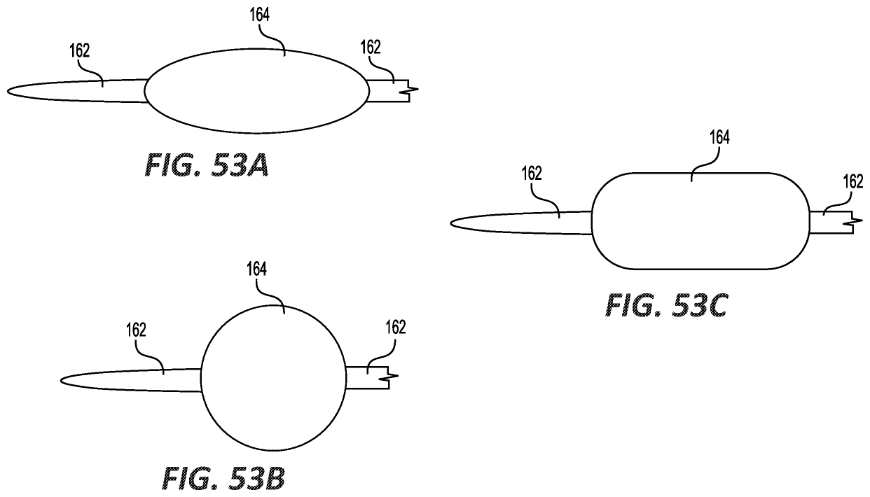

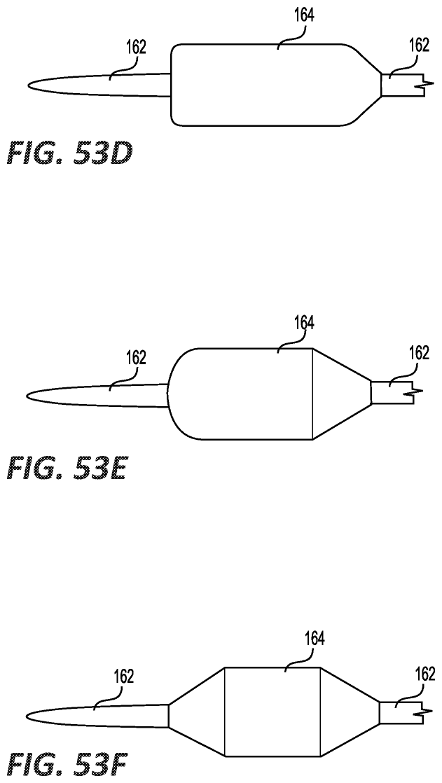

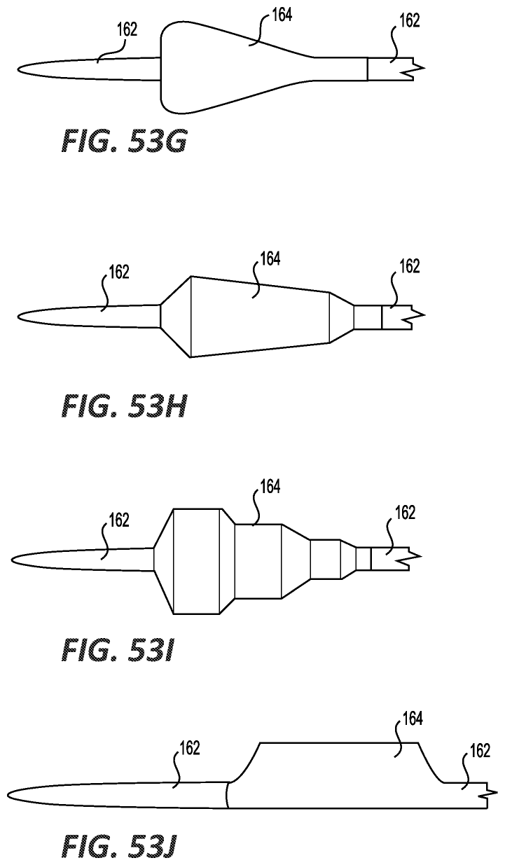

The inflated balloon 164 can have any regular and irregular shape. FIGS. 53A-53J illustrate various example shapes of the inflated balloon 164. The shape of the inflated balloon 164 can be selected based on patient anatomy and/or physician preference. As will be described below, the shape of the balloon 164 can influence the expanded shape of the distal end portion 140 of the sheath 100. The physician may select a desired shape of expanded distal end portion 140 based on the medical device being delivered and/or the tools being used, and the likelihood that withdraw/retrieval through the expanded distal opening of the sheath 100 will be necessary. FIG. 53A shows a rounded/elliptical shaped-balloon 164. FIG. 53B shows a round/spherical shaped-balloon 164. FIG. 53C shows an elongated spherical-shaped balloon 164 having an elongated cylindrical body and semi-spherical-shaped leading and trailing edges, where the leading edge is adjacent the distal end 168 of the introducer 160 and the trailing edge adjacent the proximal end 166 of the introducer 160. FIG. 53D shows an elongated conical/square-shaped balloon having an elongated cylindrical body, a square-shaped first edge and a tapered second edge. While FIG. 53D illustrates a square-shaped leading edge and a tapered trailing edge, opposite orientation is contemplated. A tapered trailing edge may be desired to provide a tapered expanded shape to the distal end portion 140 of the sheath 100. FIG. 53E shows an elongated conical/spherical-shaped balloon 164 having an elongated cylindrical body, a semi-spherical-shaped first edge tapered and a tapered second edge. While FIG. 53E illustrates a tapered trailing edge and a semi-spherical-shaped leading edge, opposite orientation is contemplated. As in FIG. 53D, the tapered trailing edge of FIG. 53E may be desired to provide a tapered expanded shape of the distal end portion 140 of the sheath 100. FIG. 53F shows an elongated conical/conical-shaped balloon including an elongated cylindrical body and tapered leading and trailing edges. The leading and trailing edges can have a corresponding taper (FIG. 53F). Alternatively, the taper of the leading edge can be greater than the taper of the trailing edge. Likewise, the taper of the trailing edge can be greater than the taper of the leading edge. It is contemplated that the balloon 164 may include only the leading and trailing edges, abutting each other, without a cylindrical portion extending therebetween. It is also contemplated that the balloon 164 can include a tapered body portion extending between the tapered leading edge and trailing edges as shown, illustrated for example, in FIG. 53H. In this example, the taper can vary between the leading edge, trailing edge and body portion. Similarly, the axial length of the taper may differ between the leading and trailing edges (and the tapered body portion where applicable). FIG. 53G shows a conical/square-shaped balloon includes a square-shaped first edge and a tapered second edge, opposite orientation is contemplated. As illustrated in FIG. 53G, a conical trailing edge may be desired to provide a conical expanded shape of the distal end portion 140 of the sheath 100). FIG. 53I shows a stepped balloon 164 including portions of varying diameter. As illustrated in FIG. 53I, the diameter of each of the portions decreases between the distal and proximal ends 168, 166 of the introducer 160, opposite orientation of the decreased diameter portions (from proximal to distal) is contemplated. FIG. 53J shows an offset balloon 146 having a height on a first side of the elongated body member 162 greater than a height on an opposite second side of the elongated body member 162. Though not shown, it is contemplated that the balloon 164 can have a square shape.

The balloon 164 can be constructed from a compliant material, a semi-compliant material, and/or a non-compliant material. For example, when the balloon 164 is composed of a compliant material, the balloon 164 may be constructed from a polyamide, polyolefin, silicone, and/or polyester. In some examples, the polyamide can be nylon. In some examples the polyolefin can be polyethylene or polypropylene. In some examples, the polyester can be polyethylene terephthalate (PET). Balloon 164 is may also be constructed from a polymeric material. Different portions of the balloon 164 may be composed of different materials, e.g., a compliant material, semi-compliant material, and/or non-compliant material. For example, a proximal portion of the balloon 164 can be composed of a less compliant material than a distal portion of the balloon 164, where the proximal portion of the balloon is adjacent the proximal end 166 of the elongated body member 162 and the distal portion of the balloon 164 is adjacent the distal end 168 of the elongated body member 162.

The balloon 164 can also be composed of an impermeable material for containing the inflation fluid, for example saline, within the balloon 164. In some examples, the inflation fluid can also include a contrast medium. The balloon 164 can also be composed of a permeable material such as a mesh or perforated material. In this example, the permeable material allows for fluid communication between an interior of the balloon 164 and an exterior of the balloon. The inflation fluid is provided to the interior of the balloon 164 via the inflation lumen 170. The inflation lumen 170 extends longitudinally within the elongated body portion 162 of the introducer 160 between a reservoir for the inflation fluid and the interior of the balloon 164. The volume of the inflation fluid can be varied to control the volume/inflation of the balloon 164. Where the balloon 164 is permeable, the flow rate of inflation fluid into the balloon 164 and through the mesh material can be varied to control the inflation of the balloon 164. Additionally, the flow rate of the inflation fluid can be controlled is such that the balloon 164 inflates to a diameter greater than the outer diameter of the elongated body member 162, even while inflation fluid is passing through the mesh material.

The balloon 164 can have a uniform wall thickness along the length and/or around the circumference of the balloon 164. In another example, the wall thickness of the balloon 164 varies along the length and/or around the circumference of the balloon 164. For example, the wall thickness of the proximal portion of the balloon 164 can be greater than the thickness of the distal portion of the balloon 164, the proximal portion of the balloon 164 adjacent the proximal end 166 of the elongated body member 162 and the distal portion of the balloon 164 is adjacent the distal end 168 of the elongated body member 162. In an alternate example, the thickness of the distal portion of the balloon 164 is greater than the thickness of the proximal portion of the balloon 164. In a further example, the balloon 164 includes at least one circumferential band of increased wall thickness.

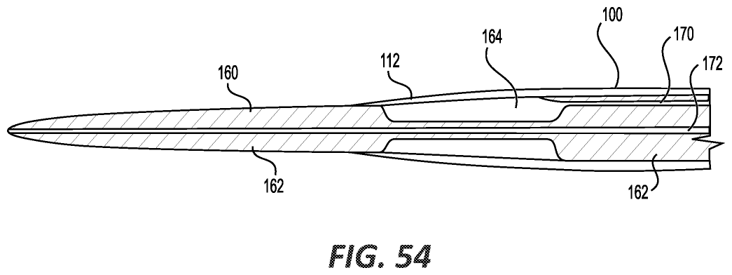

FIGS. 54-56 provide a cross sectional views of the distal end portion 140 of the sheath 100 and the introducer 160. As shown in FIG. 54, where the balloon 164 is coupled to the elongated body member 162 such that there is no break or gap between the outer surface of the elongated body member 162 and the outer surface of the balloon 164. To facilitate movement of the introducer 160 within the central lumen 112 of the sheath 100, the elongated body member 162 can be composed of a lubricious material or have a lubricious coating. In some examples, the elongated body member 162 is composed of a flexible material including, for example, high-density polyethylene, PTFE, other fluoropolymers such as ECTFE (ethylene chlorotrifluoroethylene) (e.g., Halar® available from Solvay), polyamide (e.g., nylon 6, nylon 6,6, and other nylons), acetal copolymer or polyoxymethylene (e.g., Celcon® available from Celanese), a polyolefin (e.g., HDPE) and/or polyolefin blends. Also “blends” of materials could be used, for example, a blend of HDPE and LDPE. The elongated body member 162 can include a flexibility feature/attribute to promote lateral flexibility. Example flexibility features include circumferentially and/or longitudinally extending groove(s), circumferentially/radially extending slit(s), and a coil structure embedded in our coupled to the elongated body member 162 of the introducer 160.

As illustrated in FIG. 54, the distal end of the elongated body member 162 includes a tapered tip portion adapted for insertion into a body tissue. To aid with placement of the introducer 160 and sheath 100 proximate the treatment site, the introducer can also include at least one radio opaque marker. For example, a radio opaque maker can be located proximate the tapered distal end of the introducer 160, along the elongated body member 162 at the leading end of the balloon 164, on the balloon 164, and/or along the elongated body member 162 at the trailing end of the balloon. To further help with placement of the introducer 160 and sheath 100, the introducer 160 includes a guidewire lumen 172. The guidewire lumen 172 extends through the introducer 160 and is sized and configured to receive a guidewire and facilitate advancement of the introducer 160/sheath 100 to the treatment site. As illustrated in FIG. 54, guidewire lumen 172 extends along a longitudinal centerline of the elongated body member 162, and the inflation lumen 170 is radially offset from the guidewire lumen 172 such that the inflation lumen 170 extends along a one side of the guidewire lumen 172.

The method of expanding the distal end portion 140 of the sheath 100 during delivery of a medical device is described below. FIG. 54 illustrates the sheath 100 and introducer 160 prior to and/or during insertion into the patient's vasculature, and before the distal end portion 140 of the sheath 100 is expanded by the balloon 164. As provided in FIG. 54, the introducer 160 is advanced into the central lumen 112 of the sheath 100. Generally, the outer diameter of the elongated body member 162 of the introducer is less than the diameter of the central lumen 112 of the sheath 100. Accordingly, the introducer 160 is generally movable (axially, rotationally) within the central lumen 112 of the sheath 100. However, as provided in FIG. 54, during insertion and prior to expansion of the balloon 164, at least a portion of an outer surface of the elongated body member 162 is fitted against a surface of the central lumen 112 of the introducer sheath 100 proximate the distal opening. The close fitting between the introducer 160 and the sheath 100 helps reduce push force and patient trauma during insertion of the sheath into and through the patient's tissue. The elongated body member 162 is fitted against the surface of the central lumen 112 by at least one of a press fit and/or an interference fit. As illustrated in FIG. 54, with the introducer 160 fitted to the distal end portion 140 of the sheath 100, the distal end of the introducer 160 extends through and beyond the distal opening of the central lumen 112. In some examples (not shown), the distal end of the introducer 160 is flush with the distal end/distal opening of the central lumen 112.

Typically, a guide wire is advanced into the patient's vasculature to the treatment site. With the sheath 100 and the introducer 160 coupled, the sheath 100 and introducer 160 are advanced along the guide wire to the treatment site. The guidewire can remain in place during expansion of the distal end portion 140 of the sheath 100 or it can be removed. The position of the sheath 100 and/or the introducer 160 can be imaged as it is moved through the patient's vasculature using an imaging modality such as x-ray fluoroscopy. The introducer 160 can include a radio opaque marker(s) to determine the position of the introducer 160.

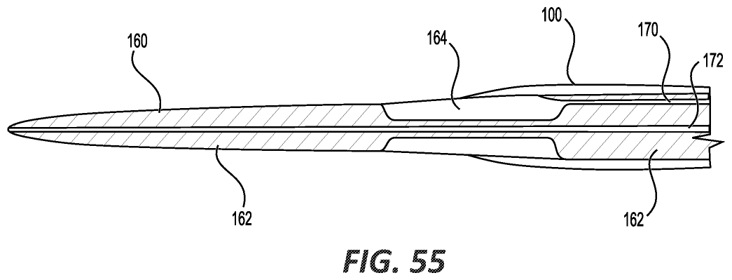

Once the sheath 100 is at the desired position, the introducer 160 is advanced axially/longitudinally within the central lumen of the sheath 100 such that an inflatable balloon 164 is axially aligned with a distal opening of the sheath 100, as shown in FIG. 55. Advancing the introducer 160 axially/longitudinally within the central lumen 112 of the sheath 100 includes advancing the distal end of the elongated body member 162 beyond the distal opening of the central lumen 112 (if it was not already extending beyond). The introducer 160 and/or sheath 100 can include a stop feature such that axial movement of the introducer 160 through the distal opening is limited to a previously determined distal-most position of the introducer 160 with respect to the sheath 100. In this distal-most position, the balloon 164 is aligned axially with the distal opening of the sheath 100. When the balloon 164 is aligned axially with the distal opening of the sheath 100 the balloon 100 is positioned such that a first portion 164A of the balloon 164 extends beyond the distal opening of the sheath 100 and a second portion 164B of the balloon 164 is positioned within the central lumen 112 of the sheath 100 (FIG. 55).

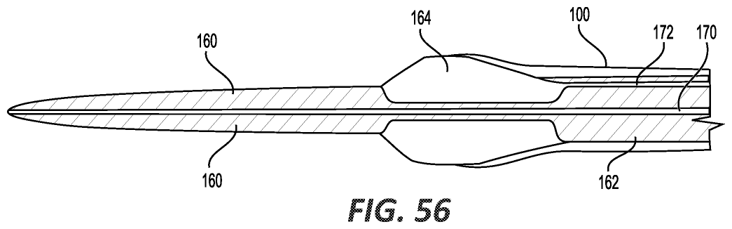

Next, as illustrated in FIG. 56, the inflation fluid is provided to the balloon 164 via the inflation lumen 170, inflating the balloon 164. The balloon 164 expands from an initial diameter to a second diameter greater than the initial diameter. Inflation of the balloon 164 results in a corresponding expansion of the distal end portion 140 of the sheath 100, such that the diameter of the distal opening increased from an initial (non-expanded) diameter to a second, larger, expanded diameter. As discussed above, the shape of the inflated balloon 146 can determine the shape of the expanded shape of the distal end portion 140 of the sheath 100. For example, as illustrated in FIG. 56 the trailing end of the balloon 164 has a tapered surface extending from an elongated cylindrical body toward the outer surface of the introducer 160. The tapered surface is located adjacent the distal opening of the sheath 100 and expands the distal end portion 140 to a corresponding tapered shape.

Next the balloon 164 is deflated as the inflation fluid is removed from the interior of the balloon 164. For example, the inflation fluid can be withdrawn from the balloon 164 via mechanical suction or vacuum. In another example, were the balloon 164 comprises a mesh material, the flow of inflation fluid into the balloon 164 can end, causing the balloon to deflate. With the balloon 164 deflated, the diameter of the balloon 164 is less than the second/inflated diameter of the balloon 164 and approaches the initial/non-inflated diameter of the balloon 164. The introducer 160 is then withdrawn through the central lumen 112 of the sheath 100 and the distal end portion 140 of the sheath 100 remains expanded after the introducer 160 has been withdrawn.

With the central lumen 112 free of the introducer 160, the medical device 12 can be advanced through the sheath 100 to the treatment site. When advancing the medical device 12 through the central lumen 112 of the sheath 100, the medical device 12 applies an outward radial force on an inner surface of the central lumen 112. This outward radial force locally expands the sheath 100 from an initial non-expanded state to a locally expanded state. The diameter of the sheath 100 increases from a first (non-expanded) diameter to a second, larger (expanded) diameter. In some examples described above, the sheath 100 includes a polymeric layer having at least one longitudinally-extending fold when the sheath 100 is at the first (non-expanded) diameter. The medical device 12 passing through the central lumen 112 of the sheath 100 applies an outward radial force on the sheath 100 causing the sheath 100 to expand radially by at least partially unfolding the longitudinally-extending fold(s). In some examples, partial unfolding of the longitudinally-extending fold(s) causes a decrease in a wall thickness of the introducer sheath 100. Similarly, in some examples described above, the sheath 100 includes a polymeric layer having a plurality of longitudinally-extending folds when the sheath 100 is at the first (non-expanded) diameter. The longitudinally-extending folds creating a plurality of circumferentially spaced ridges and a plurality of circumferentially spaced valleys. The medical device 12 passing through the central lumen 112 of the sheath 100 applies an outward radial force on the sheath 100 causing the sheath 100 to expand radially from the first (non-expanded) diameter to the second, larger (expanded) diameter by levelling out the ridges and valleys. Preferably, the medical device 12 is advanced through the sheath 100 at a push for less than the push force required for a sheath with a non-expanded tip.

Once the medical device 12 has passed, the sheath 100 locally contracts from the locally expanded state at least partially back to the non-expanded state after passage of the medical device 12. In some examples, the sheath 100 includes a layer of a self-contracting material to facilitate local contraction of the sheath.

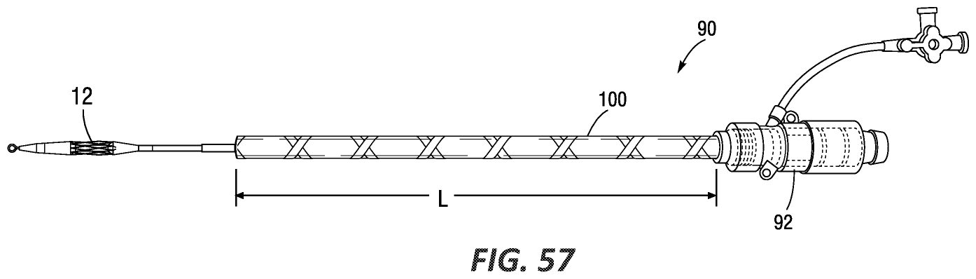

As illustrated in FIG. 57, the medical device is advanced through the central lumen 112 of the sheath 100 and beyond the distal opening for placement at the treatment site. In some examples, the medical device 12 is a prosthetic heart valve, e.g., a self-expanding prosthetic heart valve. Once the medical device 12 is delivered to the patient, the sheath 100 is removed.

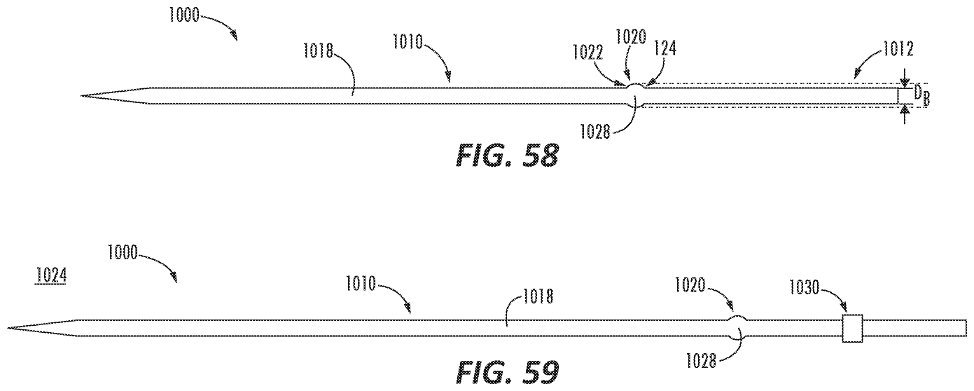

It is further contemplated that the introducer sheaths 100 described herein can be used in combination with an expansion device 1000. Example expansion devises 1000 are illustrated in FIGS. 58-64. Similar to the vessel dilator 300 and introducer 160 described above, the expansion device 1000 pre-expands the sheath and/or patient's blood vessel in advance of the delivery device. The expansion device 1000 can be an introducer or a dilator depending on when/how the expansion device 1000 is used in the procedure. For example, when used as an introducer, the expansion device 1000 is positioned within the central lumen 112 of the sheath 100 and the combined expansion device 1000 and sheath are introduced into a patient's vascular together. The expansion device 1000 can then be further advanced within the sheath to locally expand the central lumen 112 of the sheath 100. In another example, when expansion device 1000 is used as a dilator, the sheath 100 is disposed within the patient's vascular prior to the insertion of the expansion device 1000. With the sheath 100 positioned within the vasculature, the expansion device 1000 is then passed through the central lumen of the sheath 100 to dilate/expand the sheath 100 and the patient's blood vessel.

As described above, the expansion device 1000 is received within the central lumen 112 of the sheath 100. As illustrated in FIGS. 58-64, the expansion device 1000 includes an elongated body 1010 and one or more radially extending protrusions 1020 disposed along a portion of the body 1010. The expansion device 1000 is sized and configured to be received within the central lumen 112 of an expandable sheath 100 such that the protrusion 1020 at least partially expands a portion of an expandable sheath 100. As noted above, the expansion device 1000 is configured to be received within any of the of expandable sheath implementations disclosed herein. The protrusion 1020 of the expansion device 1000 can be formed from a fixed structure such that the size and shape of the protrusion 1020 does not change during use. In another example, the protrusion 1020 is constructed from an inflatable structure such that the size and shape of the protrusion 1020 can be adjusted during use.

FIG. 58 illustrates an example expansion device 1000. The expansion device 1000 includes a body 1010 with a proximal end 1012 and a tapered distal end 1014 opposite and spaced apart from the proximal end 1012 of the body 1010. The tapered distal end 1014 of the body 1010 includes a decreasing taper extending from the diameter of the body 1010 towards the distal end of the expansion device 1000. As illustrated in FIG. 58, the protrusion 1020 extends radially from the outer surface of the body 1010 such that the outer surface of the protrusion 1020 has a diameter greater than the diameter of the body 1010.

For example, the body 1010 includes a body diameter D_{B} and the protrusion 1020 includes a protrusion diameter D_{P}, and the protrusion diameter D_{P} is greater than the body diameter D_{B}. In some examples, the body diameter D_{B} is 14F, and the diameter D_{P} is greater than 14F. In other examples, the body diameter is 2F, and the protrusion diameter is greater than 2F. In other examples, the body diameter is 4F, and the protrusion diameter is greater than 4F. In other examples, the body diameter is 6F, and the protrusion diameter is greater than 6F. In other examples, the body diameter is 8F, and the protrusion diameter is greater than 8F. In other examples, the body diameter is 10F, and the protrusion diameter is greater than 10F. In other examples, the body diameter is 12F, and the protrusion diameter is greater than 12F. In other examples, the body diameter is 16F, and the protrusion diameter is greater than 16F. In other examples, the body diameter is 18F, and the protrusion diameter is greater than 18F. In other examples, the body diameter is 20F, and the protrusion diameter is greater than 20F. In other examples, the body diameter is 22F, and the protrusion diameter is greater than 22F. In other examples, the body diameter is 24F, and the protrusion diameter is greater than 24F. In other examples, the body diameter is 26F, and the protrusion diameter is greater than 26F. In other examples, the body diameter is 28F, and the protrusion diameter is greater than 28F. In other examples, the body diameter is any suitable diameter as long as the protrusion diameter is greater than the body diameter.

The protrusion 1020 can be fixedly disposed at a location along the length of the body 1010. For example, the protrusion 1020 can be fixedly coupled to and/or integrally formed with the body 1010. In some examples, the protrusion 1020 is molded to or otherwise integrally formed with the body 1010. In further examples, the protrusion 1020 is fixedly coupled to the body 1010 through an adhesive, a chemical or mechanical fastener, and or heat processing (e.g., welding).

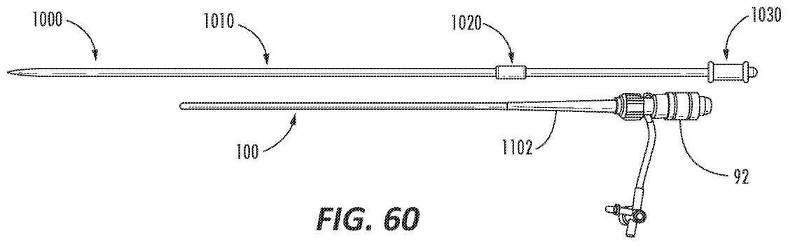

The protrusion 1020 can have any regular or irregular shape. The protrusion 1020 can have any shape similar to the balloon 164 shapes illustrated in FIGS. 53A-53J. The physician may select a desired shape of the protrusion 1020 based on the medical device being delivered and/or tools being inserted after the expansion device 1000 is used to pre-dilate and/or pre-expand the sheath 100. As shown in FIGS. 58 and 59, the protrusion 1020 can be spherically shaped with a curved leading end 1022 and curved trailing end 1024. As shown in FIG. 60, the protrusion 1020 is cylindrically shaped, with curved/tapered leading and trailing ends 1022, 1024. In other examples, and as illustrated in FIGS. 53F and 53H-53J, the protrusion 1020 has a tapered leading end 1022. As illustrated in FIGS. 53D-53J, the protrusion 1020 has a tapered trailing end 1024. In other examples, the protrusion is any suitable shape with a diameter or outer perimeter dimension greater than the body 1010 diameter that is capable of at least partially expanding a portion of the expandable sheath 100.

As illustrated in FIGS. 58-64, the protrusion 1020 is disposed between the proximal end 1012 and the tapered distal end 1014 of the body 1010. As illustrated in FIGS. 58 and 59, the protrusion 1020 is disposed adjacent the proximal end of the body 1012. In this example, insertion of the expansion device into the sheath 100 causes the protrusion 1020 to expand and/or unfold a portion of the proximal end of the sheath 100. In some examples, the sheath 100 includes a strain relief portion 1102 extending distally from the proximal end of the sheath 100 (e.g., along the outer layer/jacket) providing increased stiffness and rigidity compared to the remaining portions of the sheath 100. Moving the expansion device 1000 and protrusion 1020 through sheath 100 proximate the strain relief portion 1102 causes portion of the sheath 100 proximate the strain relief portion 1102 to at least partially expand/unfold.

Both the body 1010 and the protrusion 1020 further include an outer surface 1018, 1028. In some examples, the outer surface 1018 of the body 1010 and the outer surface 1028 of the protrusion 1020 include a hydrophilic coating to reduce friction between the sheath 100 and the expansion device 1000 and ensure that the expansion device 1000 is easily received and movable within the central lumen of the expandable sheath 100. In some examples, the hydrophilic coating includes a material with a low coefficient of friction.

In another example, as shown in FIG. 59, the expansion device 1000 further includes a locking mechanism 1030 coupled to the body 1010. The locking mechanism 1030 can be used to fix/limit the movement of the expansion device 1000 within the central lumen 112 of the sheath 100. In some examples, the locking mechanism 1030 can limit axial and/or rotational movement of the expansion device 1000 within the central lumen 112. In some examples, the locking mechanism 1030 couples to/engages with the housing 92. In further examples, the locking mechanism 1030 provides an increased diameter portion projecting from the outer surface/diameter of the sheath 100 such that the outer diameter of the locking mechanism 1030 is greater than the diameter of the central lumen 112, preventing the locking mechanism 1030 from entering the central lumen 112 and fixing the axial position of the locking mechanism 1030/expansion device 1000.

The locking mechanism 1030 is disposed along the proximal end 1012 of the body 1010 and is larger than the body diameter D_{B}. In some implementations, the locking mechanism 1030 is adjustably disposable along a portion of the body such that a user is able to adjust the distance between the protrusion 1020 and the locking mechanism 1030, thereby adjusting how far the protrusion 1020/expansion device 1000 moves within the expandable sheath 100. As a result, the locking mechanism 1030 prevents the user from inserting the expansion device 1000 (and protrusion 1020) too deep within the expandable sheath 100 and helps to avoid unnecessary trauma to a patient's vascular system.

In the example shown in FIG. 60, the expansion device 1000 can be used as an introducer. The locking mechanism 1030 is positioned at a predetermined distance from the protrusion 1020 such that, when inserted into the sheath 100, the protrusion 1020 does not extend past the strain relief portion 1102 of the sheath 100. As a result, the expansion device 1000 expands the portion of the expandable sheath 100 within the strain relief portion 1102 and the push force required to subsequently insert a medical device through the strain relief portion 1102 is greatly decreased.

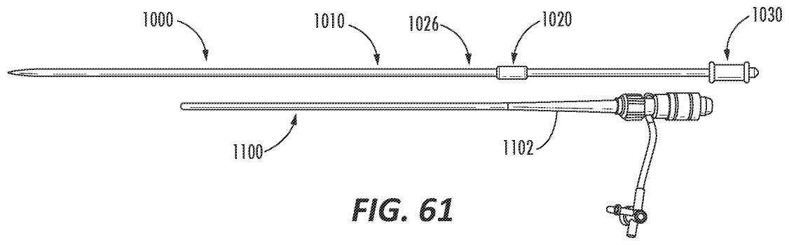

FIG. 61 illustrates another example expansion device 1000, capable of use as an introducer. The expansion devise 1000 includes a tapered leading end 1026 that extends from the outer diameter/surface of the body 1010 to the outer diameter of the protrusion 1020. In this example, when the expansion device is inserted into the central lumen 112 of the sheath 100, the protrusion 1020 and/or tapered leading edge 1026 aligns with the location of the strain relief portion 1102 of the expandable sheath 100. In the example protrusion 1020 illustrated in FIG. 60, the outer diameter of the protrusion 1020 remains constant between the proximal end of the tapered leading edge 1026 and the proximal end 1012 of the expansion device. However, as outlined above, the in other examples, the protrusion 1020 can include a trailing end having a tapered, curved or any other regular or irregular shape.

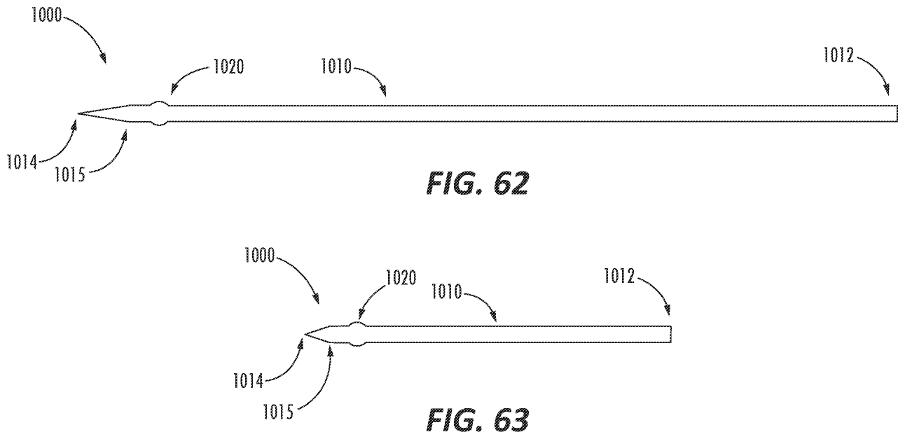

As described above, the expansion device 1000 can be used as a dilator to dilate/expand the sheath 100 and corresponding portion of the patient's blood vessel in advance of the insertion of the delivery device. FIGS. 62 and 63 illustrate an example expansion device 1000 capable of use as a dilator. In this example, the protrusion 1020 is disposed proximate the tapered distal end 1014 of the body 1010. It is contemplated that when used as a dilator, the protrusion 1020 can be located at any location between the tapered distal end 1014 of the body 1010 and the proximal end 1012 of the body 1010. As illustrated in FIGS. 62 and 63, a spherically shaped protrusion 1020 is disposed offset from the proximal end 1015 of the tapered distal tip 1014. In other examples, the protrusion is disposed adjacent the proximal end 1015 of the tapered distal end 1014. When provided at the distal end of the body 1010, the protrusion 1020 locally dilates and/or expands the sheath 100 and the patient's blood vessel as it is advanced through the central lumen of the sheath. This localized and temporary expansion reduces stress on both the sheath 100 and the blood vessel compared to typical introducers/dilators that provide a longer area of increased diameter along the sheath/vessel for a longer period of time.

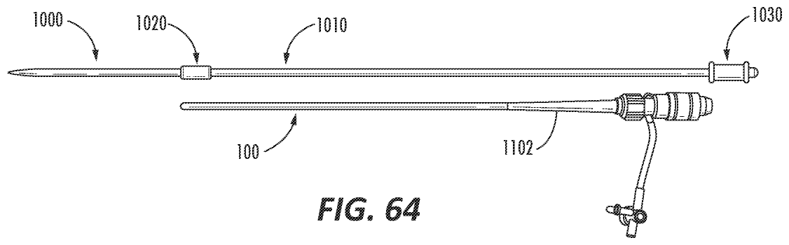

FIG. 64 illustrates another example expansion device 1000 capable of use as a dilator. In this example, the protrusion is disposed proximate the tapered distal end 1014 of the body 1010. As described above, the expansion device 1000 also includes a locking mechanism 1030. The locking mechanism 1030 is positioned relative to the protrusion 1020 of the expansion device 1000 such that the protrusion 1020 is able to extend past the strain relief portion 1102 of the corresponding expandable sheath 100 when assembled. As the expansion device 1000 and the protrusion 1020 advance through the sheath 100, the protrusion 1020 locally dilates and/or expands the sheath and the patient's blood vessel. The axial distance that the protrusion 1020 is allowed to advance within the sheath 1100 is adjustable by changing the location of the locking mechanism 1030 or, as described below, adjusting the axial location of the protrusion 1020 upon the body 1010.

As described above, the protrusion 1020 can axially and/or rotationally fixed to a certain position along/around the body 1010 of the expansion device. It is further contemplated that the axial and rotational position of the protrusion 1020 can be adjusted along/around the body 1010. For example, protrusion 1020 can include an adjustment device capable are fixedly coupling the protrusion 1020 at varying locations longitudinally and circumferentially along/around the body 1010. For example, the protrusion 1020 can include a threaded portion for engaging a corresponding threaded portion on the body 1010. In one example, the protrusion 1020 includes a central lumen/throughhole including a threaded inner surface. Likewise, the outer surface of the body 1010 can include a threaded outer surface extending along all or a portion of the outer surface of the body 1010. The threaded opening provided on the protrusion 1020 can engage the threads provided on the outer surface of the body 1010 such that the protrusion 1020 is rotationally adjustable around the body 1010 and, thus, axially movably along the body 1010 by engagement between the threaded portions. In other implementations, the protrusion 1020 is adjustably disposed on the body 1010 and includes a locking mechanism (e.g., a mechanical fastener including, for example, a pin, screw, bolt, clip, bayonet lock, or any other mechanical fastener suitable for fixing the protrusion 1020 with respect to the body 1010) for fixing the axial and rotational position of the protrusion 1020 along the body 1010.

Each of the expandable sheaths 100 shown in FIGS. 60, 61, and 64 (FIGS. 48 and 49) include an inner layer 1104 with at least one fold 1106 that is configured to move between a folded configuration, as shown in FIG. 48, and a less folded configuration, as shown in FIG. 49, during local expansion of the sheath 100. Specifically, during local expansion of the sheath 100 caused by receiving the protrusion 1020 of any of the aforementioned implementations of the expansion device 1000. As described above, examples of folded expandable sheaths can be found in U.S. application Ser. No. 12/249,867 filed Oct. 10, 2008 (issued as U.S. Pat. No. 8,690,936), U.S. application Ser. No. 13/312,739 file Dec. 6, 2011 (issued as U.S. Pat. No. 8,790,387), and U.S. Provisional Patent Application No. 62/912,569, entitled “Expandable Sheath,” which are incorporated by reference in their entireties.

A method of locally expanding an expandable sheath using an expansion device is disclosed herein. In this example, the combined sheath and expansion device can be used as an introducer. The method includes introducing an expansion device within a central lumen of the expandable sheath. The combined expandable sheath and expansion device are then advanced into a patient's vascular. The expansion device is advanced distally within the central lumen of the expandable sheath to locally expand the lumen of the sheath at a local axial location corresponding to an axial location of a radially extending protrusion provided on the expansion device.

Introducing the expansion device within the central lumen of the expandable sheath also includes positioning the expansion device within the central lumen of the sheath such that the radially extending protrusion is disposed proximally (outside of) the strain relief portion of the sheath. In another example, introducing the expansion device within the central lumen of the expandable sheath includes positioning the expansion device within the central lumen of the sheath such that the protrusion is at least partially disposed within the strain relief portion of the expandable sheath.

In some examples, locally expanding the lumen of the sheath further includes advancing the expansion device through the strain relief portion of the sheath and into an elongated body portion of the sheath. The expansion device can then be advanced beyond the strain relief portion of the sheath and into an elongated body portion of the sheath.

The method next includes removing the expansion device from the expandable sheath such that the outer diameter of the sheath (and inner diameter of the central lumen of the sheath) corresponding to the location(s) of the protrusion is greater than the non-expanded, initial, diameter of the sheath.

Lastly, the method includes inserting a medical device into the central lumen of the expandable sheath.

Another method of locally expanding an expandable sheath using an expansion device is disclosed herein. In this example, the sheath and expansion device can be used as a dilator to pre-dilate/expand the sheath and the patient's vasculature. The method includes introducing an expandable sheath having a central lumen into a patient's vascular. The expansion device is advanced into the central lumen of the expandable sheath. The expansion device is advanced distally within the central lumen of the expandable sheath to locally expand the lumen of the sheath at an (local) axial location corresponding to the axial location of the radially extending protrusion provided on the expansion device.

Introducing an expansion device within the central lumen of the expandable sheath can further include positioning the expansion device within the central lumen of the sheath such that the radially extending protrusion is at least disposed within a strain relief portion of the expandable sheath.

In some examples, locally expanding the lumen of the sheath further includes advancing the expansion device beyond the strain relief portion of the sheath and into an elongated body portion of the sheath.

The method next includes removing the expansion device from the expandable sheath such that the outer diameter of the sheath (and inner diameter of the central lumen of the sheath) corresponding to the location(s) of the protrusion is greater than the non-expanded, initial, diameter of the sheath. Lastly, the method includes inserting a medical device into the central lumen of the expandable sheath.

General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of a valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

Unless otherwise indicated, all numbers expressing dimensions, quantities of components, molecular weights, percentages, temperatures, forces, times, and so forth, as used in the specification or claims, are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing examples from discussed prior art, the example numbers are not approximates unless the word “about” is recited. Furthermore, not all alternatives recited herein are equivalents.

In view of the many possible examples to which the principles of the disclosed technology may be applied, it should be recognized that the illustrated examples are only preferred examples and should not be taken as limiting the scope of the disclosure. Rather, the scope of the disclosure is at least as broad as the following claims. We therefore claim all that comes within the scope and spirit of these claims.

### Exemplary Aspects

Example 1: An expandable introducer comprising: an elongated body member; an inflatable balloon disposed between a proximal and distal end of the elongated body member, the balloon expandable from a deflated configuration to an inflated configuration; and an inflation lumen in fluid communication with the inflatable balloon, the inflation sized and configured for providing an inflation fluid to the balloon; wherein, in the deflated configuration, an outer diameter of the balloon corresponds to an outer diameter of the elongated body member, and in the inflated configuration, the outer dimeter of the balloon is greater than the outer diameter of the elongated body member, wherein at least a portion of the balloon is sized and configured to pass through a distal opening of an expandable introducer sheath when the balloon is in the deflated condition, the balloon sized and configured to expand at least a portion of a distal end of an introducer sheath as the balloon is inflated.

Example 2: The expandable introducer according to any example herein, particularly example 1, including an expandable introducer sheath for deploying a medical device, wherein the elongated body member is received within a central lumen of the introducer sheath, and axially and rotatably movable therein.

Example 3: The expandable introducer according to any example herein, particularly example 2, wherein the elongated body member is movable through a distal opening provided in the introducer sheath.

Example 4: The expandable introducer according to any example herein, particularly example 3, wherein during insertion into the patient's vasculature, at least a portion of an outer surface of the elongated body member is fitted against a surface of the central lumen of the introducer sheath proximate the distal opening.

Example 5: The expandable introducer according to any example herein, particularly example 4, wherein the elongated body member is fitted against the surface of the central lumen by at least one of a press fit and an interference fit.

Example 6: The expandable introducer according to any example herein, particularly examples 1-5, wherein axial movement of the elongated body member through the distal opening is limited such that when the elongated body member is provided in a distal-most position, the balloon is aligned axially with the distal opening of the introducer sheath.

Example 7: The expandable introducer according to any example herein, particularly example 6, wherein when the elongated body member is at the distal-most position, a first portion of the balloon extends beyond the distal opening of the introducer sheath and a second portion of the balloon remains within the central lumen of the introducer sheath, wherein the second portion of the balloon is sized and configured to expand at least a portion of the distal end of the introducer sheath as the balloon expands from the deflated configuration to the inflated configuration.

Example 8: The expandable introducer according to any example herein, particularly example 7, wherein the second portion of the balloon includes a tapered surface adapted to dilate the at least a portion of the distal end of the introducer sheath to a corresponding tapered shape.

Example 9: The expandable introducer according to any example herein, particularly examples 1-8, wherein a diameter of the central lumen of the introducer sheath is greater than the diameter of the elongated body member.

Example 10: The expandable introducer according to any example herein, particularly examples 1-9, wherein a diameter of the distal opening of the introducer sheath when dilated is up to about 75% larger than a diameter of the distal opening of the introducer sheath when not dilated.

Example 11: The expandable introducer according to any example herein, particularly examples 1-10, wherein when a medical device is passed through the central lumen of the introducer sheath, a diameter of the introducer sheath expands from a first diameter to a second, larger, diameter.

Example 12: The expandable introducer according to any example herein, particularly example 11, wherein the introducer sheath includes at least one polymeric layer including a plurality of longitudinally-extending folds when the sheath is at the first diameter, wherein a medical device passing through the central lumen of the introducer sheath applies an outward radial force on the introducer sheath causing the introducer sheath to expand radially from the first diameter to the second diameter by at least partially unfolding the plurality of longitudinally-extending folds.

Example 13: The expandable introducer according to any example herein, particularly example 12, wherein the at least partial unfolding of the plurality of longitudinally-extending folds causes a decrease in a wall thickness of the introducer sheath.

Example 14: The expandable introducer according to any example herein, particularly example 11, wherein the introducer sheath includes at least one polymeric layer including a plurality of longitudinally-extending folds when the sheath is at the first diameter, the longitudinally-extending folds creating a plurality of circumferentially spaced ridges and a plurality of circumferentially spaced valleys, wherein a medical device passing through the central lumen of the introducer sheath applies an outward radial force on the introducer sheath causing the introducer sheath to expand radially from the first diameter to the second diameter by levelling out the ridges and valleys.

Example 15: The expandable introducer according to any example herein, particularly example 12, wherein the introducer sheath includes at least one layer of a self-contracting material.

Example 16: The expandable introducer according to any example herein, particularly examples 1-15, wherein the outer diameter of the elongated body member is less than a diameter of the central lumen.

Example 17: The expandable introducer according to any example herein, particularly examples 1-16, wherein the elongated body member comprises a lubricious material.

Example 18: The expandable introducer according to any example herein, particularly examples 1-17, wherein the elongated body member is flexible.

Example 19: The expandable introducer according to any example herein, particularly example 18, wherein the elongated body member composed of a flexible material including high-density polyethylene.

Example 20: The expandable introducer according to any example herein, particularly example 18, wherein the elongated body member includes a flexibility feature including at least one circumferentially and/or longitudinally extending groove, a slit, and a coil.

Example 21: The expandable introducer according to any example herein, particularly examples 1-20, wherein the distal end of the elongated body member includes a tapered tip portion adapted for insertion into a body tissue.

Example 22: The expandable introducer according to any example herein, particularly examples 1-21, wherein the introducer includes a radio opaque marker.

Example 23: The expandable introducer according to any example herein, particularly example 22, wherein the radio opaque maker is located proximate at least one of the tapered distal end of the elongated body member, along the elongated body member proximate a leading end of the balloon, on the balloon, and along the elongated body member at a trailing end of the balloon.

Example 24: The expandable introducer according to any example herein, particularly examples 1-23, including a guidewire lumen extending therethrough.

Example 25: The expandable introducer according to any example herein, particularly example 24, wherein the guidewire lumen extends along a longitudinal centerline of the elongated body member, wherein the inflation lumen is radially offset from the guidewire lumen, the inflation lumen extending along a first side of the guidewire lumen.

Example 26: The expandable introducer according to any example herein, particularly examples 1-25, wherein a shape of the balloon in the inflated configuration comprises at least one of a regular or irregular shape.

Example 27: The expandable introducer according to any example herein, particularly examples 1-26, wherein a shape of the balloon in the inflated configuration includes a tapered leading edge and a tapered trailing edge, the leading edge adjacent the distal end of the elongated body member and the trailing edge adjacent the proximal end of the elongated body member.

Example 28: The expandable introducer according to any example herein, particularly example 27, wherein the taper of the leading edge corresponds to the taper of the trailing edge.

Example 29: The expandable introducer according to any example herein, particularly example 27, wherein the taper of the leading edge is greater than the taper of the trailing edge.

Example 30: The expandable introducer according to any example herein, particularly example 27, wherein the taper of the leading edge is less than the taper of the trailing edge.

Example 31: The expandable introducer according to any example herein, particularly example 27, wherein the balloon includes a cylindrical body portion extending between the tapered leading edge and the tapered trailing edge.

Example 32: The expandable introducer according to any example herein, particularly example 27, wherein the balloon includes a tapered body portion extending between the tapered leading edge and the tapered trailing edge, wherein the taper of the body portion varies from the tapers of each of the leading and trailing edges.

Example 33: The expandable introducer according to any example herein, particularly examples 1-32, wherein a shape of the balloon in the inflated configuration comprises at least one an elliptical-shaped balloon, a spherical-shaped balloon, a square-shaped balloon, a conical-shaped balloon, an elongated spherical-shaped balloon, an elongated conical/square-shaped balloon, an elongated conical/spherical-shaped balloon, an elongated conical/conical-shaped balloon, a conical/square-shaped balloon, a tapered balloon, a stepped balloon, an offset balloon.

Example 34: The expandable introducer according to any example herein, particularly example 33, wherein the conical-shaped balloon includes a tapered leading and trailing edge, wherein the elongated spherical-shaped balloon includes an elongated cylindrical body portion and a semi-spherical-shaped leading and trailing edge, wherein the elongated conical/square-shaped balloon includes an elongated cylindrical body, a first edge tapered and a second edge square-shaped, wherein the elongated conical/spherical-shaped balloon includes an elongated cylindrical body, a first edge tapered and a second edge semi-spherical-shaped, wherein the elongated conical/conical-shaped balloon includes a tapered leading and trailing edge, wherein the conical/square-shaped balloon includes a tapered first edge and a square-shaped second edge, wherein the tapered balloon includes a tapered leading edge and a tapered trailing edge, wherein the stepped balloon includes portions of varying diameter, wherein the offset balloon includes a height on a first side of the elongated body member greater than a height on an opposite second side of the elongated body member.

Example 35: The expandable introducer according to any example herein, particularly example 1, wherein the balloon is made of polymeric material.

Example 36: The expandable introducer according to any example herein, particularly examples 1-35, wherein the balloon is composed of at least one of a compliant material, a semi-compliant material, and a non-compliant material.

Example 37: The expandable introducer according to any example herein, particularly example 36, wherein the balloon is composed of a compliant material.

Example 38: The expandable introducer according to any example herein, particularly example 37, wherein the balloon is composed of at least one of polyolefin, silicone, polyethylene terephthalate.

Example 39: The expandable introducer according to any example herein, particularly example 36, wherein different portions of the balloon are composed of different ones of the compliant material, semi-compliant material, and non-compliant material.

Example 40: The expandable introducer according to any example herein, particularly example 39, wherein a proximal portion of the balloon is composed of a less compliant material than a distal portion of the balloon, wherein the proximal portion of the balloon is adjacent the proximal end of the elongated body member and the distal portion of the balloon is adjacent the distal end of the elongated body member.

Example 41: The expandable introducer according to any example herein, particularly example 40, wherein at least a portion of the proximal portion of the balloon includes a tapered surface.

Example 42: The expandable introducer according to any example herein, particularly example 41, wherein the tapered surface sized and configured to be located adjacent a distal opening of an introducer sheath such that the tapered surface of the balloon is adapted to expand the distal end of an introducer sheath.

Example 43: The expandable introducer according to any example herein, particularly examples 1-42, wherein the balloon is composed of an impermeable material.

Example 44: The expandable introducer according to any example herein, particularly examples 1-43, wherein the balloon is composed of a permeable material.

Example 45: The expandable introducer according to any example herein, particularly example 44, wherein the balloon comprises a mesh material that allows for fluid communication between an interior of the balloon and an exterior of the balloon.

Example 46: The expandable introducer according to any example herein, particularly example 45, where if the flow rate of inflation fluid through the mesh material of the balloon is such that the balloon inflates to have a diameter greater than the outer diameter of the elongated body member.

Example 47: The expandable introducer according to any example herein, particularly examples 1-46, including an inflation fluid comprises saline.

Example 48: The expandable introducer according to any example herein, particularly examples 1-47, wherein an inflation fluid includes a contrast medium.

Example 49: The expandable introducer according to any example herein, particularly examples 1-48, wherein the balloon has a uniform thickness along a length and/or around a circumference of the balloon.

Example 50: The expandable introducer according to any example herein, particularly examples 1-49, wherein a thickness of the balloon varies along a length and/or around a circumference of the balloon.

Example 51: The expandable introducer according to any example herein, particularly example 50, wherein a thickness of a proximal portion of the balloon is greater than a thickness of a distal portion of the balloon, wherein the proximal portion of the balloon is adjacent the proximal end of the elongated body member and the distal portion of the balloon is adjacent the distal end of the elongated body member.

Example 52: The expandable introducer according to any example herein, particularly example 50, wherein a thickness of a distal portion of the balloon is greater than a thickness of a proximal portion of the balloon, wherein the proximal portion of the balloon is adjacent the proximal end of the elongated body member and the distal portion of the balloon is adjacent the distal end of the elongated body member.

Example 53: The expandable introducer according to any example herein, particularly example 50, wherein the balloon includes at least one circumferential band of increased thickness.

Example 54: The expandable introducer according to any example herein, particularly examples 1-53, wherein the balloon is coupled to the elongated body member such that there is no break between an outer surface of the elongated body member and an outer surface of the balloon.

Example 55: The expandable introducer according to any example herein, particularly examples 1-54, wherein, in the inflated configuration, the diameter of the balloon is up to about 75% larger than the diameter of the elongated body member.

Example 56: An introducer sheath system comprising: an expandable introducer sheath for deploying a medical device; an introducer received within a central lumen of the introducer sheath and axially and rotatably movable therein, the introducer comprising: an elongated body member having a proximal end and a tapered distal end; an inflatable balloon disposed between the proximal end and the distal end of the elongated body member, the balloon expandable from a deflated configuration to an inflated configuration; and an inflation lumen in fluid communication with the inflatable balloon, the inflation sized and configured for providing an inflation fluid to the balloon; wherein, in the deflated configuration, an outer diameter of the balloon corresponds to an outer diameter of the elongated body member, and in the inflated configuration, the outer dimeter of the balloon is greater than the outer diameter of the elongated body member, wherein at least a portion of the balloon is sized and configured to pass through a distal opening of the introducer sheath when the balloon is in the deflated condition, as the balloon is inflated at least a portion of the distal end of an introducer sheath expands, increasing a diameter of the distal opening.

Example 57: The introducer sheath system according to any example herein, particularly example 56, wherein the elongated body member is movable through the distal opening provided in the introducer sheath.

Example 58: The introducer sheath system according to any example herein, particularly example 57, wherein during insertion into the patient's vasculature, at least a portion of an outer surface of the elongated body member is fitted against a surface of the central lumen of the introducer sheath proximate the distal opening.

Example 59: The introducer sheath system according to any example herein, particularly example 58, wherein the elongated body member is fitted against the surface of the central lumen by at least one of a press fit and an interference fit.

Example 60: The introducer sheath system according to any example herein, particularly examples 56-59, wherein axial movement of the elongated body member through the distal opening is limited such that when the elongated body member is provided in a distal-most position, the balloon is aligned axially with the distal opening of the introducer sheath.

Example 61: The introducer sheath system according to any example herein, particularly example 60, wherein when the elongated body member is at the distal-most position, a first portion of the balloon extends beyond the distal opening of the introducer sheath and a second portion of the balloon remains within the central lumen of the introducer sheath, wherein the second portion of the balloon is sized and configured to dilate at least a portion of the distal end of the introducer sheath as the balloon expands from the deflated configuration to the inflated configuration.

Example 62: The introducer sheath system according to any example herein, particularly example 61, wherein the second portion of the balloon includes a tapered surface adapted to dilate the at least a portion of the distal end of the introducer sheath to a corresponding tapered shape.

Example 63: The introducer sheath system according to any example herein, particularly examples 56-62, wherein a diameter of the central lumen of the introducer sheath is greater than the diameter of the elongated body member.

Example 64: The introducer sheath system according to any example herein, particularly examples 56-63, wherein a diameter of the distal opening of the introducer sheath when dilated up to about 75% larger than a diameter of the distal opening of the introducer sheath when not dilated.

Example 65: The introducer sheath system according to any example herein, particularly examples 56-64, wherein when a medical device is passed through the central lumen of the introducer sheath, a diameter of the introducer sheath expands from a first diameter to a second, larger, diameter.

Example 66: The introducer sheath system according to any example herein, particularly example 65, wherein the introducer sheath includes at least one polymeric layer including a plurality of longitudinally-extending folds when the sheath is at the first diameter, wherein a medical device passing through the central lumen of the introducer sheath applies an outward radial force on the introducer sheath causing the introducer sheath to expand radially from the first diameter to the second diameter by at least partially unfolding the plurality of longitudinally-extending folds.

Example 67: The introducer sheath system according to any example herein, particularly example 66 wherein the at least partial unfolding of the plurality of longitudinally-extending folds causes a decrease in a wall thickness of the introducer sheath.

Example 68: The introducer sheath system according to any example herein, particularly examples 65-67, wherein the introducer sheath includes at least one polymeric layer including a plurality of longitudinally-extending folds when the sheath is at the first diameter, the longitudinally-extending folds creating a plurality of circumferentially spaced ridges and a plurality of circumferentially spaced valleys, wherein a medical device passing through the central lumen of the introducer sheath applies an outward radial force on the introducer sheath causing the introducer sheath to expand radially from the first diameter to the second diameter by levelling out the ridges and valleys.

Example 69: The introducer sheath system according to any example herein, particularly examples 65-68, wherein the introducer sheath includes at least one layer of a self-contracting material.

Example 70: The introducer sheath system according to any example herein, particularly examples 56-69, wherein the outer diameter of the elongated body member ranges between is less than a diameter of the central lumen of the introducer sheath.

Example 71: The introducer sheath system according to any example herein, particularly examples 56-70, wherein the elongated body member comprises a lubricious material.

Example 72: The introducer sheath system according to any example herein, particularly example 61, wherein the elongated body member is flexible.

Example 73: The introducer sheath system according to any example herein, particularly example 72, wherein the elongated body member composed of a flexible material including high-density polyethylene.

Example 74: The introducer sheath system according to any example herein, particularly example 72, wherein the elongated body member includes a flexibility feature including at least one circumferentially and/or longitudinally extending groove, a slit, and a coil.

Example 75: The introducer sheath system according to any example herein, particularly examples 56-74, wherein the distal end of the elongated body member includes a tapered tip portion adapted for insertion into a body tissue.

Example 76: The introducer sheath system according to any example herein, particularly examples 56-75, wherein the introducer includes a radio opaque marker.

Example 77: The introducer sheath system according to any example herein, particularly example 76, wherein the radio opaque maker is located proximate at least one of the tapered distal end of the elongated body member, along the elongated body member proximate a leading end of the balloon, on the balloon, and along the elongated body member at a trailing end of the balloon.

Example 78: The introducer sheath system according to any example herein, particularly examples 56-77, including a guidewire lumen extending therethrough.

Example 79: The introducer sheath system according to any example herein, particularly example 78, wherein the guidewire lumen extends along a longitudinal centerline of the elongated body member, wherein the inflation lumen is radially offset from the guidewire lumen, the inflation lumen extending along a first side of the guidewire lumen.

Example 80: The introducer sheath system according to any example herein, particularly examples 56-79, wherein a shape of the balloon in the inflated configuration comprises at least one of a regular or irregular shape.

Example 81: The introducer sheath system according to any example herein, particularly examples 56-80, wherein a shape of the balloon in the inflated configuration includes a tapered leading edge and a tapered trailing edge, the leading edge adjacent the distal end of the elongated body member and the trailing edge adjacent the proximal end of the elongated body member.

Example 82: The introducer sheath system according to any example herein, particularly example 81, wherein the taper of the leading edge corresponds to the taper of the trailing edge.

Example 83: The introducer sheath system according to any example herein, particularly example 81, wherein the taper of the leading edge is greater than the taper of the trailing edge.

Example 84: The introducer sheath system according to any example herein, particularly example 81, wherein the taper of the leading edge is less than the taper of the trailing edge.

Example 85: The introducer sheath system according to any example herein, particularly example 81, wherein the balloon includes a cylindrical body portion extending between the tapered leading edge and the tapered trailing edge.

Example 86: The introducer sheath system according to any example herein, particularly example 81, wherein the balloon includes a tapered body portion extending between the tapered leading edge and the tapered trailing edge, wherein the taper of the body portion varies from the tapers of each of the leading and trailing edges.

Example 87: The introducer sheath system according to any example herein, particularly examples 56-86, wherein a shape of the balloon in the inflated configuration comprises at least one an elliptical-shaped balloon, a spherical-shaped balloon, a square-shaped balloon, a conical-shaped balloon, an elongated spherical-shaped balloon, an elongated conical/square-shaped balloon, an elongated conical/spherical-shaped balloon, an elongated conical/conical-shaped balloon, a conical/square-shaped balloon, a tapered balloon, a stepped balloon, an offset balloon.

Example 88: The introducer sheath system according to any example herein, particularly example 87, wherein the conical-shaped balloon includes a tapered leading and trailing edge, wherein the elongated spherical-shaped balloon includes an elongated cylindrical body portion and a semi-spherical-shaped leading and trailing edge, wherein the elongated conical/square-shaped balloon includes an elongated cylindrical body, a first edge tapered and a second edge square-shaped, wherein the elongated conical/spherical-shaped balloon includes an elongated cylindrical body, a first edge tapered and a second edge semi-spherical-shaped, wherein the elongated conical/conical-shaped balloon includes a tapered leading and trailing edge, wherein the conical/square-shaped balloon includes a tapered first edge and a square-shaped second edge, wherein the tapered balloon includes a tapered leading edge and a tapered trailing edge, wherein the stepped balloon includes portions of varying diameter, wherein the offset balloon includes a height on a first side of the elongated body member greater than a height on an opposite second side of the elongated body member.

Example 89: The introducer sheath system according to any example herein, particularly examples 56-88, wherein the balloon is made of polymeric material.

Example 90: The introducer sheath system according to any example herein, particularly examples 56-89, wherein the balloon is composed of at least one of a compliant material, a semi-compliant material, and a non-compliant material.

Example 91: The introducer sheath system according to any example herein, particularly example 90, wherein the balloon is composed of a compliant material.

Example 92: The introducer sheath system according to any example herein, particularly example 91, wherein the balloon is composed of at least one of polyolefin, silicone, polyethylene terephthalate.

Example 93: The introducer sheath system according to any example herein, particularly example 90, wherein different portions of the balloon are composed of different ones of the compliant material, semi-compliant material, and non-compliant material.

Example 94: The introducer sheath system according to any example herein, particularly example 93, wherein a proximal portion of the balloon is composed of a less compliant material than a distal portion of the balloon, wherein the proximal portion of the balloon is adjacent the proximal end of the elongated body member and the distal portion of the balloon is adjacent the distal end of the elongated body member.

Example 95: The introducer sheath system according to any example herein, particularly example 94, wherein at least a portion of the proximal portion of the balloon includes a tapered surface.

Example 96: The introducer sheath system according to any example herein, particularly example 95, wherein the tapered surface sized and configured to be located adjacent a distal opening of an introducer sheath such that the tapered surface of the balloon is adapted dilate the distal end of an introducer sheath.

Example 97: The introducer sheath system according to any example herein, particularly examples 56-96, wherein the balloon is composed of an impermeable material.

Example 98: The introducer sheath system according to any example herein, particularly examples 56-97, wherein the balloon is composed of a permeable material.

Example 99: The introducer sheath system according to any example herein, particularly example 98, wherein the balloon comprises a mesh material that allows for fluid communication between an interior of the balloon and an exterior of the balloon.

Example 100: The introducer sheath system according to any example herein, particularly example 99, where if the flow rate of inflation fluid through the mesh material of the balloon is such that the balloon inflates to have a diameter greater than the outer diameter of the elongated body member.

Example 101: The introducer sheath system according to any example herein, particularly examples 56-100, including an inflation fluid comprises saline.

Example 102: The introducer sheath system according to any example herein, particularly example 101, wherein the inflation fluid includes a contrast medium.

Example 103: The introducer sheath system according to any example herein, particularly examples 56-102, wherein the balloon has a uniform thickness along a length and/or around a circumference of the balloon.

Example 104: The introducer sheath system according to any example herein, particularly examples 56-103, wherein a thickness of the balloon varies along a length and/or around a circumference of the balloon.

Example 105: The introducer sheath system according to any example herein, particularly example 104, wherein a thickness of a proximal portion of the balloon is greater than a thickness of a distal portion of the balloon, wherein the proximal portion of the balloon is adjacent the proximal end of the elongated body member and the distal portion of the balloon is adjacent the distal end of the elongated body member.

Example 106: The introducer sheath system according to any example herein, particularly example 104, wherein a thickness of a distal portion of the balloon is greater than a thickness of a proximal portion of the balloon, wherein the proximal portion of the balloon is adjacent the proximal end of the elongated body member and the distal portion of the balloon is adjacent the distal end of the elongated body member.

Example 107: The introducer sheath system according to any example herein, particularly example 104, wherein the balloon includes at least one circumferential band of increased thickness.

Example 108: The introducer sheath system according to any example herein, particularly examples 56-107, wherein the balloon is coupled to the elongated body member such that there is no break between an outer surface of the elongated body member and an outer surface of the balloon.

Example 109: The introducer sheath system according to any example herein, particularly examples 56-108, wherein, in the inflated configuration, the diameter of the balloon is up to about 75% larger than the diameter of the elongated body member.

Example 110: The introducer sheath system according to any example herein, particularly example 61, wherein the maximum diameter of the balloon in the inflated configuration is about 50% larger than the diameter of the elongated body member.

Example 111: A method of pre-dilating an introducer sheath tip comprising: positioning an expandable introducer within a central lumen of an expandable sheath, the introducer including: an elongated body member; an inflatable balloon disposed between a proximal and distal end of the elongated body member, the balloon expandable from a deflated configuration to an inflated configuration, when in the deflated configuration an initial diameter of the balloon corresponds to an outer diameter of the elongated body member, when in the inflated configuration, the inflated dimeter of the balloon is greater than the outer diameter of the elongated body member; and an inflation lumen in fluid communication with the inflatable balloon, the inflation sized and configured for providing an inflation fluid to the balloon; advancing the introducer axially within the central lumen of the sheath such that the inflatable balloon is axially aligned with a distal opening of the sheath; inflating the balloon to the inflated diameter, where the inflated diameter of the balloon is greater than an initial diameter of the distal opening and thereby expanding a diameter of the distal opening of the sheath; deflating the balloon; and withdrawing the introducer from the central lumen of the sheath.

Example 112: The method according to any example herein, particularly example 111, wherein positioning the introducer within the central lumen of the sheath includes fitting at least a portion of an outer surface of the introducer against a surface of the central lumen of the sheath proximate the distal opening.

Example 113: The method according to any example herein, particularly example 112, wherein the introducer is fitted against the surface of the central lumen by at least one of a press fit and an interference fit.

Example 114: The method according to any example herein, particularly examples 111-113, wherein positioning the introducer within the central lumen of the sheath includes positioning the introducer such that the distal end of the elongated body member of the introducer extends through and beyond the distal opening of the central lumen.

Example 115: The method according to any example herein, particularly examples 111-114, wherein advancing the introducer axially within the central lumen of the sheath includes advancing the distal end of the elongated body member beyond the distal opening of the central lumen, wherein axial movement of the elongated body member through the distal opening is limited such that when the elongated body member is provided in a distal-most position the balloon is aligned axially with the distal opening of the introducer sheath.

Example 116: The method according to any example herein, particularly examples 111-115, wherein advancing the introducer axially includes positioning the balloon such that a first portion of the balloon extends beyond the distal opening of the sheath and a second portion of the balloon is positioned within the central lumen of the sheath.

Example 117: The method according to any example herein, particularly example 116, wherein the second portion of the balloon includes a tapered surface adapted to expand the at least a portion of the distal end of the sheath to a corresponding tapered shape.

Example 118: The method according to any example herein, particularly examples 111-117, wherein the diameter of the distal opening of the introducer sheath when expanded is up to about 75% larger than a diameter of the distal opening of the introducer sheath when not expanded.

Example 119: A method of delivering a medical device comprising: inserting an expandable sheath and an expandable introducer at least partially into the vasculature of the patient, the introducer received within a central lumen of the sheath; advancing the introducer axially within the central lumen of the sheath such that an inflatable balloon disposed on an elongated body member of the introducer is axially aligned with a distal opening of the sheath; inflating the balloon to a diameter greater than an initial diameter of the distal opening and thereby expanding a diameter of the distal opening of the sheath; deflating the balloon; withdrawing the introducer from the central lumen of the sheath; advancing a medical device through the central lumen of the sheath; and delivering the medical device to the patient.

Example 120: The method according to any example herein, particularly example 119, wherein during insertion, at least a portion of an outer surface of the introducer is fitted against a surface of the central lumen of the sheath proximate the distal opening.

Example 121: The method according to any example herein, particularly example 120, wherein the introducer is fitted against the surface of the central lumen by at least one of a press fit and an interference fit.

Example 122: The method according to any example herein, particularly examples 119-121, wherein the introducer is received within the central lumen of the sheath such that a distal end of the introducer extends through and beyond the distal opening of the central lumen.

Example 123: The method according to any example herein, particularly examples 119-122, wherein advancing the introducer axially within the central lumen of the sheath includes advancing the distal end of the elongated body member beyond the distal opening of the central lumen, wherein axial movement of the elongated body member through the distal opening is limited such that when the elongated body member is provided in a distal-most position the balloon is aligned axially with the distal opening of the introducer sheath.

Example 124: The method according to any example herein, particularly examples 119-123, wherein advancing the introducer axially includes positioning the balloon such that a first portion of the balloon extends beyond the distal opening of the expandable sheath and a second portion of the balloon is positioned within the central lumen of the sheath.

Example 125: The method according to any example herein, particularly examples 119-124, wherein the diameter of the distal opening of the introducer sheath when expanded is up to about 75% larger than a diameter of the distal opening of the introducer sheath when not expanded.

Example 126: The method according to any example herein, particularly examples 119-125, further comprising: inserting a guidewire at least partially into the vasculature of the patient; advancing the introducer over the guidewire to a treatment position within the patient's vasculature.

Example 127: The method according to any example herein, particularly example 126, further comprising withdrawing the guidewire from the introducer.

Example 128: The method according to any example herein, particularly examples 119-127, further comprising: visualizing the position of at least one of the expandable sheath and the introducer within the vasculature using an imaging modality, wherein the introducer includes a radio opaque marker located proximate at least one of the tapered distal end, a leading end of the balloon, and a trailing end of the balloon.

Example 129: The method according to any example herein, particularly examples 119-128, wherein inflating the balloon includes providing an inflation fluid to the balloon via an inflation lumen extending though the introducer in fluid communication in an interior volume of the balloon.

Example 130: The method according to any example herein, particularly examples 119-129, wherein the balloon includes a tapered surface adapted to expand the at least a portion of the distal end of the sheath to a corresponding tapered shape.

Example 131: The method according to any example herein, particularly examples 119-130, wherein withdrawing the withdrawing the introducer from the central lumen of the sheath includes moving the introducer axially towards a proximal end of the sheath until the introducer is completely removed from the central lumen.

Example 132: The method according to any example herein, particularly examples 119-131, wherein the diameter of the distal opening of the sheath remains expanded after the balloon is deflated and the introducer withdrawn.

Example 133: The method according to any example herein, particularly examples 119-132, wherein advancing a medical device through the central lumen of the sheath further comprises: advancing the medical device through the central lumen of the sheath as the medical device applies an outward radial force on an inner surface of the central lumen; locally expanding the sheath from an initial non-expanded state to a locally expanded state; at least partially unfolding a plurality of longitudinally-extending folds in the sheath during local expansion of the sheath; and locally collapsing the sheath from the locally expanded state at least partially back to the collapsed state after passage of the medical device.

Example 134: The method according to any example herein, particularly examples 119-133, wherein advancing a medical device through the central lumen of the sheath further comprises advancing the medical device at a push force less than the push force required for a sheath with a non-expanded tip.

Example 135: The method according to any example herein, particularly examples 119-134, wherein delivering the medical device to the patient further comprises advancing the medical device through the distal opening of the sheath for placement at a treatment site.

Example 136: The method according to any example herein, particularly examples 119-135, wherein the medical device is a prosthetic heart valve.

Example 137: The method according to any example herein, particularly example 136, wherein the prosthetic heart valve comprises a self-expanding heart valve.

Example 138: The method according to any example herein, particularly example 136, wherein the prosthetic heart valve comprises a balloon-expanding heart valve.

Example 139: The method according to any example herein, particularly examples 119-138, further comprising: removing the sheath from the patient after the medical device is delivered to the patient.

Example 140: An expansion device that is configured to be received within an expandable sheath, the device comprising: a body comprising an outer surface, a proximal end, and a tapered distal end opposite and spaced apart from the proximal end of the body; a radially extending protrusion disposed along a portion of the body, the radially extending protrusion having an outer surface, the radially extending protrusion has a diameter greater than a diameter of the body, and wherein the device is sized and configured to be received within a central lumen of an expandable sheath such that the radially extending protrusion at least partially expands a portion of the expandable sheath.

Example 141: The expansion device according to any example herein, particularly example 140, wherein the outer surface of the body and the outer surface of the radially extending protrusion include a hydrophilic coating.

Example 142: The expansion device according to any example herein, particularly example 141, wherein the hydrophilic coating comprises a material with a low coefficient of friction.

Example 143: The expansion device according to any example herein, particularly examples 140-142, wherein the radially extending protrusion is fixedly disposed at a location along the length of the body.

Example 144: The expansion device according to any example herein, particularly example 143, wherein the radially extending protrusion is molded to the outer surface of the body.

Example 145: The expansion device according to any example herein, particularly example 143, wherein the radially extending protrusion is fixedly disposed to the body through an adhesive, fasteners, or welding.

Example 146: The expansion device according to any example herein, particularly examples 140-142, wherein the radially extending protrusion is adjustably disposed at a location along the length of the body.

Example 147: The expansion device according to any example herein, particularly example 146, wherein the outer surface of the body comprises a first plurality of threads that extend along a portion of the length of the body, wherein the radially extending protrusion comprises an inner surface including a second plurality of threads that correspond to the first plurality of threads such that the radially extending protrusion is configured to be rotationally adjustable around the body, wherein the radially extending protrusion is axially movable along the body by engagement between first and second plurality of threads.

Example 148: The expansion device according to any example herein, particularly examples 140-147, wherein the diameter of the body is 2F, and the diameter of the radially extending protrusion is greater than 2F.

Example 149: The expansion device according to any example herein, particularly examples 140-147, wherein the diameter of the body is 4F, and the diameter of the radially extending protrusion is greater than 4F.

Example 150: The expansion device according to any example herein, particularly examples 140-147, wherein the diameter of the body is 6F, and the diameter of the radially extending protrusion is greater than 6F.

Example 151: The expansion device according to any example herein, particularly examples 140-147, wherein the diameter of the body is 8F, and the diameter of the radially extending protrusion is greater than 8F.

Example 152: The expansion device according to any example herein, particularly examples 140-147, wherein the diameter of the body is 10F, and the diameter of the radially extending protrusion is greater than 10F.

Example 153: The expansion device according to any example herein, particularly examples 140-147, wherein the diameter of the body is 12F, and the diameter of the radially extending protrusion is greater than 12F.

Example 154: The expansion device according to any example herein, particularly examples 140-147, wherein the diameter of the body is 14F, and the diameter of the radially extending protrusion is greater than 14F.

Example 155: The expansion device according to any example herein, particularly examples 140-147, wherein the diameter of the body is 16F, and the diameter of the radially extending protrusion is greater than 16F.

Example 156: The expansion device according to any example herein, particularly examples 140-147, wherein the diameter of the body is 20F, and the diameter of the radially extending protrusion is greater than 20F.

Example 157: The expansion device according to any example herein, particularly examples 140-147, wherein the diameter of the body is 24F, and the diameter of the radially extending protrusion is greater than 24F.

Example 158: The expansion device according to any example herein, particularly examples 140-147, wherein the diameter of the body is 28F, and the diameter of the radially extending protrusion is greater than 28F.

Example 159: The expansion device according to any example herein, particularly examples 140-158, wherein the radially extending protrusion is disposed between the proximal end and the tapered distal end of the body.

Example 160: The expansion device according to any example herein, particularly example 159, wherein the radially extending protrusion is disposed adjacent the tapered distal end of the body.

Example 161: The expansion device according to any example herein, particularly example 160, wherein the radially extending protrusion is offset from a proximal end of the tapered distal tip.

Example 162: The expansion device according to any example herein, particularly examples 140-159, wherein the radially extending protrusion is disposed adjacent the proximal end of the body.

Example 163: The expansion device according to any example herein, particularly example 162, wherein the radially extending protrusion is offset from the proximal end of the body.

Example 164: The expansion device according to any example herein, particularly examples 140-163, wherein the radially extending protrusion is spherically shaped.

Example 165: The expansion device according to any example herein, particularly examples 140-163, wherein the radially extending protrusion is cylindrically shaped.

Example 166: The expansion device according to any example herein, particularly examples 140-165, wherein radially extending protrusion has a curved leading end and curved trailing end.

Example 167: The expansion device according to any example herein, particularly examples 140-166, wherein the radially extending protrusion has a tapered leading end.

Example 168: The expansion device according to any example herein, particularly examples 140-167, wherein the radially extending protrusion has a tapered trailing end.

Example 169: The expansion device according to any example herein, particularly examples 140-168, wherein the device is a dilator.

Example 170: The expansion device according to any example herein, particularly examples 140-168, wherein the device is an introducer.

Example 171: The expansion device according to any example herein, particularly example 170, further comprising a locking mechanism disposed along the proximal end of the body.

Example 172: The expansion device according to any example herein, particularly example 171, wherein a diameter of the locking mechanism is larger than the diameter of the body.

Example 173: The expansion device according to any example herein, particularly example 171 or 172, wherein the locking mechanism is adjustably disposable along at least a portion of the length of the body

Example 174: A sheath system comprising: an expandable sheath comprising: an inner layer defining a central lumen of the sheath; an outer layer extending at least partially around the inner layer, wherein the inner layer and outer layer transition from a non-expanded and an expanded configuration; an expansion device movable within the central lumen of the sheath, the device comprising: a body including an outer surface, a proximal end and a tapered distal end opposite and spaced apart from the proximal end; a radially extending protrusion disposed along a portion of the body, the radially extending protrusion having an outer surface having a diameter greater than a diameter of the body, wherein receipt of the expansion device within the central lumen of the sheath causes the sheath to locally expand at least a portion of the sheath in response to the outwardly directed radial force provided by the radially extending protrusion.

Example 175: The sheath system according to any example herein, particularly example 174, wherein removal of expansion device from the central lumen of the outer layer causes the outer layer to locally contract from the expanded configuration at least partially back to the non-expanded configuration.

Example 176: The sheath system according to any example herein, particularly examples 174 or 175, wherein the inner layer includes a folded portion configured to move between a folded configuration and a less folded configuration during local expansion of the sheath.

Example 177: The sheath system according to any example herein, particularly example 176, wherein the folded portion comprises a first folded region and a second folded region and an overlapping portion extending between the first and second regions, wherein the first folded region is configured to move closer to the second folded region to shorten the overlapping portion at a local axial location during application of a radial outward force by passage of the expansion device and wherein shortening of the overlapping portion corresponds with a local expansion of the lumen.

Example 178: The sheath system according to any example herein, particularly example 177, wherein the first folded region is configured to move further away from the second folded region to lengthen the overlapping portion at the local axial location after removal of the radial outward force and wherein lengthening of the overlapping portion corresponds with a local contraction of the lumen.

Example 179: The sheath system according to any example herein, particularly examples 177 or 178, wherein the first folded region and the second folded region are circumferentially spaced from each other, wherein the overlapping portion extends circumferentially between the first and second folded regions.

Example 180: The sheath system according to any example herein, particularly examples 177-179, wherein the inner layer defines a circumferentially continuous layer and the overlapping portion is radially spaced from an outer surface of a non-overlapping portion of the inner layer; wherein the outer layer defines a discontinuous outer layer including an underlying portion radially spacing the overlapping portion away from the outer surface of the non-overlapping portion.

Example 181: The sheath system according to any example herein, particularly examples 174-180, wherein the expandable sheath further includes: an elastic outer cover extending around the outer layer exerting a radially inward force on the inner and outer layers.

Example 182: The sheath system according to any example herein, particularly examples 174-181, wherein the outer surface of the body and the radially extending protrusion of the expansion device include a hydrophilic coating.

Example 183: The sheath system according to any example herein, particularly examples 174-182, wherein the radially extending protrusion is fixedly disposed at a location along the length of the body.

Example 184: The sheath system according to any example herein, particularly examples 174-182, wherein the radially extending protrusion is adjustably disposed at a location along the length of the body.

Example 185: The sheath system according to any example herein, particularly example 184, wherein the outer surface of the body comprises a first plurality of threads that extend along a portion of the length of the body, wherein the radially extending protrusion comprises an inner surface including a second plurality of threads that correspond to the first plurality of threads such that the radially extending protrusion is configured to be rotationally adjustable around the length of the body, wherein the radially extending protrusion is axially movable along the body by engagement between first and second plurality of threads.

Example 186: The sheath system according to any example herein, particularly examples 174-185, wherein the expandable sheath includes a strain relief portion adjacent a proximal end of the sheath and an elongated body portion extending between the strain relief portion and a distal end of the sheath, the strain relief portion having a larger diameter than the elongated body portion of the sheath, wherein the diameter of the radially extending protrusion is greater than the diameter of the strain relief portion.

Example 187: The sheath system according to any example herein, particularly example 186, wherein a distal end of the strain relief portion includes a decreasing taper between the diameter of the strain relief portion and the diameter of the elongated body portion.

Example 188: The sheath system according to any example herein, particularly examples 174-187, wherein the expansion device further includes a locking mechanism for engaging the sheath and fixing the axial position of the expansion device within the central lumen of the sheath.

Example 189: The sheath system according to any example herein, particularly example 188, wherein the expandable sheath is coupled to a sheath hub at the proximal end, wherein the locking mechanism engages the sheath hub to fix the axial position of the expansion device within the central lumen of the sheath.

Example 190: A method of locally expanding an expandable sheath comprising: introducing an expansion device within a central lumen of the expandable sheath; introducing the combined expandable sheath and expansion device into a patient's vascular; advancing the expansion device distally within the central lumen of the expandable sheath to locally expand the lumen of the sheath at a local axial location corresponding to an axial location of a radially extending protrusion provided on the expansion device.

Example 191: The method according to any example herein, particularly example 190, wherein introducing an expansion device within the central lumen of the expandable sheath includes: positioning the expansion device within the central lumen of the sheath such that the radially extending protrusion is at least disposed within a strain relief portion of the expandable sheath.

Example 192: The method according to any example herein, particularly example 191, wherein locally expanding the lumen of the sheath further comprises: advancing the expansion device beyond the strain relief portion of the sheath and into an elongated body portion of the sheath.

Example 193: The method according to any example herein, particularly examples 190-192, further comprising removing the expansion device from the expandable sheath such that the outer diameter of the sheath is greater than the non-expanded, initial, diameter of the sheath.

Example 194: The method according to any example herein, particularly example 193, further comprising inserting a medical device into the central lumen of the expandable sheath.

Example 195: A method of locally expanding an expandable sheath comprising: introducing an expandable sheath into a patient's vascular, the expandable sheath having a central lumen; introducing the expansion device into the central lumen of the expandable sheath; advancing the expansion device distally within the central lumen of the expandable sheath to locally expand the lumen of the sheath at a local axial location corresponding to an axial location of a radially extending protrusion provided on the expansion device.

Example 196: The method according to any example herein, particularly example 195, wherein introducing an expansion device within the central lumen of the expandable sheath includes: positioning the expansion device within the central lumen of the sheath such that the radially extending protrusion is at least disposed within a strain relief portion of the expandable sheath.

Example 197: The method according to any example herein, particularly example 196, wherein locally expanding the lumen of the sheath further comprises: advancing the expansion device beyond the strain relief portion of the sheath and into an elongated body portion of the sheath.

Example 198: The method according to any example herein, particularly examples 195-197, further comprising removing the expansion device from the expandable sheath such that the outer diameter of the sheath is greater than the non-expanded, initial, diameter of the sheath.

Example 199: The method according to any example herein, particularly example 198, further comprising inserting a medical device into the central lumen of the expandable sheath.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

1. An expandable introducer comprising:
an elongated body member;
an inflatable balloon disposed between a proximal and distal end of the elongated body member, the balloon expandable from a deflated configuration to an inflated configuration; and
an inflation lumen in fluid communication with the inflatable balloon, the inflation sized and configured for providing an inflation fluid to the balloon;
wherein, in the deflated configuration, an outer diameter of the balloon corresponds to an outer diameter of the elongated body member, and in the inflated configuration, the outer dimeter of the balloon is greater than the outer diameter of the elongated body member,
wherein at least a portion of the balloon is sized and configured to pass through a distal opening of an expandable introducer sheath when the balloon is in the deflated condition, the balloon sized and configured to expand at least a portion of a distal end of an introducer sheath as the balloon is inflated.

2. The expandable introducer of claim 1, wherein axial movement of the elongated body member through the distal opening is limited such that when the elongated body member is provided in a distal-most position, the balloon is aligned axially with the distal opening of the introducer sheath,
wherein when the elongated body member is at the distal-most position, a first portion of the balloon extends beyond the distal opening of the introducer sheath and a second portion of the balloon remains within the central lumen of the introducer sheath,
wherein the second portion of the balloon is sized and configured to expand at least a portion of the distal end of the introducer sheath as the balloon expands from the deflated configuration to the inflated configuration.

3. The expandable introducer of claim 2, wherein the second portion of the balloon includes a tapered surface adapted to dilate the at least a portion of the distal end of the introducer sheath to a corresponding tapered shape.

4. The expandable introducer of claim 1, wherein the introducer sheath includes at least one polymeric layer including a plurality of longitudinally-extending folds when the sheath is at the first diameter,
wherein a medical device passing through the central lumen of the introducer sheath applies an outward radial force on the introducer sheath causing the introducer sheath to expand radially from the first diameter to the second diameter by at least partially unfolding the plurality of longitudinally-extending folds.

5. The expandable introducer of claim 1, wherein the elongated body member includes a flexibility feature including at least one circumferentially and/or longitudinally extending groove, a slit, and a coil.

6. The expandable introducer of claim 1, wherein the introducer includes a radio opaque marker, wherein the radio opaque maker is located proximate at least one of the tapered distal ends of the elongated body member, along the elongated body member proximate a leading end of the balloon, on the balloon, and along the elongated body member at a trailing end of the balloon.

7. The expandable introducer of claim 1, wherein a shape of the balloon in the inflated configuration comprises at least one an elliptical-shaped balloon, a spherical-shaped balloon, a square-shaped balloon, a conical-shaped balloon, an elongated spherical-shaped balloon, an elongated conical/square-shaped balloon, an elongated conical/spherical-shaped balloon, an elongated conical/conical-shaped balloon, a conical/square-shaped balloon, a tapered balloon, a stepped balloon, an offset balloon.

8. The expandable introducer of claim 7, wherein the conical-shaped balloon includes a tapered leading and trailing edge,
wherein the elongated spherical-shaped balloon includes an elongated cylindrical body portion and a semi-spherical-shaped leading and trailing edge,
wherein the elongated conical/square-shaped balloon includes an elongated cylindrical body, a first edge tapered and a second edge square-shaped,
wherein the elongated conical/spherical-shaped balloon includes an elongated cylindrical body, a first edge tapered and a second edge semi-spherical-shaped,
wherein the elongated conical/conical-shaped balloon includes a tapered leading and trailing edge,
wherein the conical/square-shaped balloon includes a tapered first edge and a square-shaped second edge,
wherein the tapered balloon includes a tapered leading edge and a tapered trailing edge,
wherein the stepped balloon includes portions of varying diameter,
wherein the offset balloon includes a height on a first side of the elongated body member greater than a height on an opposite second side of the elongated body member.

9. The expandable introducer of claim 1, wherein a proximal portion of the balloon is composed of a less compliant material than a distal portion of the balloon, wherein the proximal portion of the balloon is adjacent the proximal end of the elongated body member and the distal portion of the balloon is adjacent the distal end of the elongated body member.

10. An introducer sheath system comprising:
an expandable introducer sheath for deploying a medical device;
an introducer received within a central lumen of the introducer sheath and axially and rotatably movable therein, the introducer comprising:
an elongated body member having a proximal end and a tapered distal end;
an inflatable balloon disposed between the proximal end and the distal end of the elongated body member, the balloon expandable from a deflated configuration to an inflated configuration; and
an inflation lumen in fluid communication with the inflatable balloon, the inflation sized and configured for providing an inflation fluid to the balloon;
wherein, in the deflated configuration, an outer diameter of the balloon corresponds to an outer diameter of the elongated body member, and in the inflated configuration, the outer dimeter of the balloon is greater than the outer diameter of the elongated body member,
wherein at least a portion of the balloon is sized and configured to pass through a distal opening of the introducer sheath when the balloon is in the deflated condition, as the balloon is inflated at least a portion of the distal end of an introducer sheath expands, increasing a diameter of the distal opening.

11. The introducer system of claim 10, wherein when the elongated body member is at the distal-most position, a first portion of the balloon extends beyond the distal opening of the introducer sheath and a second portion of the balloon remains within the central lumen of the introducer sheath,
wherein the second portion of the balloon is sized and configured to dilate at least a portion of the distal end of the introducer sheath as the balloon expands from the deflated configuration to the inflated configuration.

12. The introducer system of claim 11, wherein the second portion of the balloon includes a tapered surface adapted to dilate the at least a portion of the distal end of the introducer sheath to a corresponding tapered shape.

13. The introducer system of claim 10, wherein the introducer sheath includes at least one polymeric layer including a plurality of longitudinally-extending folds when the sheath is at the first diameter,
wherein a medical device passing through the central lumen of the introducer sheath applies an outward radial force on the introducer sheath causing the introducer sheath to expand radially from the first diameter to the second diameter by at least partially unfolding the plurality of longitudinally-extending folds.

14. The introducer system of claim 10, wherein a shape of the balloon in the inflated configuration comprises at least one an elliptical-shaped balloon, a spherical-shaped balloon, a square-shaped balloon, a conical-shaped balloon, an elongated spherical-shaped balloon, an elongated conical/square-shaped balloon, an elongated conical/spherical-shaped balloon, an elongated conical/conical-shaped balloon, a conical/square-shaped balloon, a tapered balloon, a stepped balloon, an offset balloon.

15. The introducer system of claim 14, wherein the conical-shaped balloon includes a tapered leading and trailing edge,
wherein the elongated spherical-shaped balloon includes an elongated cylindrical body portion and a semi-spherical-shaped leading and trailing edge,
wherein the elongated conical/square-shaped balloon includes an elongated cylindrical body, a first edge tapered and a second edge square-shaped,
wherein the elongated conical/spherical-shaped balloon includes an elongated cylindrical body, a first edge tapered and a second edge semi-spherical-shaped,
wherein the elongated conical/conical-shaped balloon includes a tapered leading and trailing edge,
wherein the conical/square-shaped balloon includes a tapered first edge and a square-shaped second edge,
wherein the tapered balloon includes a tapered leading edge and a tapered trailing edge,
wherein the stepped balloon includes portions of varying diameter,
wherein the offset balloon includes a height on a first side of the elongated body member greater than a height on an opposite second side of the elongated body member.

16. The introducer system of claim 10, wherein a proximal portion of the balloon is composed of a less compliant material than a distal portion of the balloon, wherein the proximal portion of the balloon is adjacent the proximal end of the elongated body member and the distal portion of the balloon is adjacent the distal end of the elongated body member.

17. An expansion device that is configured to be received within an expandable sheath, the device comprising:
a body comprising an outer surface, a proximal end, and a tapered distal end opposite and spaced apart from the proximal end of the body;
a radially extending protrusion disposed along a portion of the body, the radially extending protrusion having an outer surface, the radially extending protrusion has a diameter greater than a diameter of the body, and
wherein the device is sized and configured to be received within a central lumen of an expandable sheath such that the radially extending protrusion at least partially expands a portion of the expandable sheath.

18. A sheath system comprising:
an expandable sheath comprising:
an inner layer defining a central lumen of the sheath;
an outer layer extending at least partially around the inner layer,
wherein the inner layer and outer layer transition from a non-expanded and an expanded configuration;
an expansion device movable within the central lumen of the sheath, the device comprising:
a body including an outer surface, a proximal end and a tapered distal end opposite and spaced apart from the proximal end;
a radially extending protrusion disposed along a portion of the body, the radially extending protrusion having an outer surface having a diameter greater than a diameter of the body,
wherein receipt of the expansion device within the central lumen of the sheath causes the sheath to locally expand at least a portion of the sheath in response to the outwardly directed radial force provided by the radially extending protrusion.
